SEQUENCE LISTING (7 sequences)

SEQ ID NO: 1 - DNA, 13 bp, Homo sapiens
atttatttattta

SEQ ID NO: 2 - DNA, 13 bp, Homo sapiens
ctttatttattta

SEQ ID NO: 3 - DNA, 13 bp, Homo sapiens
aattatttatact

SEQ ID NO: 4 - DNA, 13 bp, Homo sapiens
tttaatttattaa

SEQ ID NO: 5 - DNA, 13 bp, Homo sapiens
ttttatttatttt

SEQ ID NO: 6 - DNA, 13 bp, Homo sapiens
atttatttacatt

SEQ ID NO: 7 - DNA, 13 bp, Homo sapiens
aaatatttaaaat

(19) United States
(12) Patent Application Publication
(10) Pub. No.: US 2020/0101057 A1
(43) Pub. Date: Apr. 2, 2020

(54) COMBINATION THERAPY AGAINST CHEMORESISTANCE IN LEUKEMIA

(71) Applicant: The General Hospital Corporation, Boston, MA (US)

(72) Inventors: Shobha Vasudevan, Boston, MA (US); Sooncheol Lee, Quincy, MA (US)

(21) Appl. No.: 16/498,605

(22) PCT Filed: Mar. 28, 2018
(86) PCT No.: PCT/US18/24926

§ 371 Date: Sep. 27, 2019

Related U.S. Application Data
(60) Provisional application No. 62/477,757, filed on Mar. 28, 2017.
(60) Provisional application No. 62/637,027, filed on Mar. 1, 2018.

(51) Int. Cl. A61K 31/4418; A61K 31/437; G01N 33/574

(52) U.S. Cl. CPC A61K 31/4418; G01N 2800/52; G01N 33/57488; A61K 31/437

(57) ABSTRACT

Compositions and methods for targeting chemoresistant cells in leukemia using combination therapies.

### CLAIM OF PRIORITY

This application claims the benefit of U.S. Provisional Application Ser. No. 62/477,757, filed on Mar. 28, 2017 and 62/637,027, filed on Mar. 1, 2018. The entire contents of the foregoing are incorporated herein by reference.

### FEDERALLY SPONSORED RESEARCH OR DEVELOPMENT

This invention was made with Government support under Grant Nos. GM100202 and CA185086 awarded by the National Institutes of Health. The Government has certain rights in the invention.

### TECHNICAL FIELD

Described herein are compositions and methods for targeting chemoresistant cells in leukemia, e.g., in acute myelogenous leukemia (AML), using combination therapies.

### BACKGROUND

Quiescent (G0) cells are an assortment of reversibly arrested cells, including dormant stem cells, that are found as a clinically relevant subpopulation in cancers (1-3), (4-9). Such cells are anti-proliferative, anti-differentiation, and anti-apoptotic, and show distinct properties including resistance to harsh conditions (10-14), (1, 2, 4-7, 15-26).

### SUMMARY

G0 cells show specific gene expression that may underlie their resistance and other distinct properties (11, 14), (4-7, 15-26). Analyses from multiple groups revealed some genes up-regulated at the transcriptional level (3, 16, 27). However, altered polyadenylation site selection on mRNAs produces longer 3′-untranslated regions (3′-UTRs) in G0 cells, compared to proliferating cells, which increases the potential for more 3′-UTR regulatory elements that can mediate gene expression regulation (28-30). These data indicate involvement of post-transcriptional regulation of gene expression in G0 cancer cells. Translation mechanisms are distinct in G0 leukemic cells, with decreased canonical translation mechanisms and increase in mRNA translation by alternative mechanisms that involve non-canonical translation initiation factors (31) and 3′-UTR mediated specific mRNA translation (32). An altered translation profile independent of transcript level changes was also observed in immortalized G0 fibroblasts (33). These data suggested that alternate mechanisms in G0 leukemic cells might regulate a distinct translatome (mRNAs associated with and translated on polysomes) to mediate their resistance properties. The translated gene profiles, their mechanisms and outcomes on cancer persistence remain to be investigated. Therefore, we analyzed the translatome and proteome of chemotherapy-surviving G0 cancer cells, to provide comprehensive information to complement and expand upon previous transcriptome analyses (3-7, 16, 27, 34-38), and reveal critical genes that are specifically, translationally regulated for cell survival in these conditions.

G0 can be induced by growth factor-deprivation or serum-starvation in distinct cell types, and by other conditions that isolate dormant cancer stem cells (11, 12, 12-14) (13, 39). Our data demonstrate that serum-starvation induced G0 THP1 cells are chemoresistant, similar to chemosurviving leukemic cells, isolated after chemotherapy. Chemoresistant cells isolated via serum-starvation or as chemosurviving cells after chemotherapy show inhibition of the canonical translation mechanism. These data suggest alternative translation of specific mRNAs when these cells are chemoresistant. Consistently, the translatomes and proteomes of serum-starved G0 and chemosurviving cells show similarity, indicating that a specific translation program is common to serum-starvation induced G0 cells that exhibit resistance, and to chemosurviving cells—which may in part, underlie their common property of chemoresistance.

We found that chemotherapy and serum-starvation induced DNA damage response (DDR) and stress signaling-p38 MAPK and endoplasmic reticulum (ER) stress—that lead to upregulation of specific genes via post-transcriptional and translational mechanisms. These are important signaling pathways in stress response and disease (40-42) (43) (44-49) (50-57). Our data revealed AU-rich elements (AREs) enriched in mRNAs that are upregulated in G0 and in chemosurviving (resistant) cells—due to p38 MAPK stress signaling mediated activation (58-66) of MK2 (67-73). MK2 regulates an ARE binding mRNA decay and translation repression factor, Zinc finger 36 homolog or Tristetraprolin (ZFP36 or TTP) (67-74) (75-81). We found that p38 MAPK/MK2 mediated regulation of TTP, along with regulation of other ARE binding factors and decay complexes, led to increased ARE bearing mRNA levels and translation in chemoresistant G0 cells. Our data uncovered that stress signaling-ER, ATM (DNA damage induced ATM kinase), and p38 MAPK mediated STAT1/interferon pathway (82-86)—reduced key rate-limiting steps of canonical translation mechanisms in chemoresistant cells, which permitted specific gene translation by non-canonical mechanisms that were observed previously in G0 (31, 32). The post-transcriptionally/translationally regulated genes expressed in G0 resistant cells, include pro-inflammatory genes of the TNFα/NFκB pathway-which were required for cell survival and thus for chemoresistance in leukemic cells and patient samples. Consistently, we could decrease expression of TNFα, and thus reduce chemoresistance, by expressing a non-regulatable, active mutant TTP (67-73). Significantly, other upregulated genes included immune modulators and interferon response genes that are associated with chemoresistance, as well as chemokines that induce immune cell migration (73, 87-96), (97) (82, 85) (98-102). Notably, correlating with their early induction in serum-starved G0 cells or chemotherapy treated cells, pharmacological inhibition of stress signaling or key translated inflammatory response genes (that increase cell survival genes and alter cells early on)—prior to or along with chemotherapy-significantly reduced chemoresistance in cancer cell lines, in vivo AML mouse model, and in patient samples. As shown herein, DNA damage and stress signaling caused post-transcriptional and translational alterations to produce a specialized gene expression program of pro-inflammatory, immune effectors that elicit chemoresistance and cancer cell survival.

Thus, provided herein are methods for reducing resistance to chemotherapy in a subject who has cancer, the method comprising administering to the subject an effective amount of a p38 MAPK inhibitor prior to administering chemotherapy, e.g., at least one dose of a p38 MAPK inhibitor prior to administering a first dose of chemotherapy.

Also provided herein are methods for identifying whether a subject who has cancer is in need of a treatment for reducing resistance to chemotherapy, e.g., a treatment as described herein. The methods include providing a sample comprising cells from the cancer in the subject; detecting a level of phosphorylated Tristetraprolin (phospho-TTP) in the sample; comparing the level of phospho-TTP in the sample to a reference level of phospho-TTP; identifying a subject who has a level of phospho-TTP above the reference level as being in need of a treatment for reducing resistance to chemotherapy, and optionally administering to the subject an effective amount of a p38 MAPK inhibitor prior to administering chemotherapy.

In some embodiments, the cancer is a leukemia, e.g., acute myelogenous leukemia (AML), or a solid cancer, e.g., breast.

In some embodiments, the p38 MAPK inhibitor is selected from the group consisting of SB203580; Doramapimod (BIRB 796); SB202190 (FHPI); Ralimetinib (LY2228820); VX-702; PH-797804; VX-745; TAK-715; Pamapimod (R-1503, Ro4402257); BMS-582949; SB239063; Losmapimod (GW856553X); Skepinone-L; Pexmetinib (ARRY-614); Hydroxyquinazoline; AL 8697; AMG 548; AMG-47a; ARRY-797; CGH 2466 dihydrochloride; CMPD-1; CV-65; D4476 DBM 1285 dihydrochloride; EO 1428; JX-401; Losmapimod/GW856533X; ML 3403; p38/SAPK2 Inhibitor (SB 202190); Pamapimod; PD 169,316; R1487; Saquayamycin B1; SB 202190; SB 203580; SB 706504; SB202190 Hydrochloride); SB203580; SB220025; SB239063; SB242235; SCIO-323, SCIO 469; SD-169; SKF 86002 dihydrochloride; SX 011; TA 01; TA 02; TAK 715; VX-745; or VX-702.

In some embodiments, the methods include administering an effective amount of pirfenidone with the p38 MAPK inhibitor, and/or administering a cholesterol inhibitor, e.g., a statin.

In some embodiments, at least one dose of the p38 MAPK inhibitor is administered 2-24 hours before a first dose of chemotherapy, and optionally wherein additional doses of the p38 MAPK inhibitor are administered concurrently with (e.g., at the same time as, or 1-24 hours before, each additional dose of) chemotherapy.

In some embodiments, the cancer is acute myelogenous leukemia (AML).

Also provided herein are pharmaceutical compositions comprising a p38MAPK inhibitor and pirfenidone, and a pharmaceutically acceptable carrier, and compositions comprising a p38MAPK inhibitor and pirfenidone for use in a method of treating a subject who has cancer. In some embodiments, the p38 MAPK inhibitor is Ralimetinib (LY2228820).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1Q. SS and AraCS cells show reversible cell cycle arrest, chemoresistance, and a similar proteome. A. Experimental design for global profiling of G0 resistant cells. G0 chemoresistant cells were induced from S+ cells by SS or AraC treatment. Transcriptome, translatome and proteome of S+, SS, and AraCS cells were analyzed by comparative microarray or quantitative proteomics (Tandem-mass-Tag mass spectrometry) analyses. B-D. Cell cycle/proliferation analysis in S+ and SS cells using B. BrdU and PI staining, C. Western analyses of p27 KIP1 (p27) and Hes1, and D. Levels of polysome associated KI67 mRNA are shown. E & G. Reversible cell cycle arrest of SS and AraCS cells. Cell counting after trypan blue cell viability staining shows that cell proliferation is inhibited upon SS and 5 mM AraC treatment compared to S+ cells. However, cells proliferate again when serum is added to SS cells (SS→S+), or when AraCS cells are washed with PBS and resuspended in fresh RPMI media having 10% serum (AraCS→S+). F & H. Chemoresistance of SS and AraCS cells. Cell counting after trypan blue staining was performed to assess the IC50 value and cell viability of S+, SS and AraCS cells treated with various concentrations of AraC for 3 days. IC50 was also measured in AraCS cells washed with PBS and resuspended in fresh RPMI media having 10% serum (AraCS→S+). I. Western blotting analysis of phospho-eIF2a and total eIF2a in S+, SS and AraCS cells. J. Polysome profiles of SS and AraCS cells compared to S+ cells are shown. The mRNAs bound to heavy polysome fractions (>3 ribosomes) of S+, SS, and AraCS cells were analyzed by microarray. K. Correlation between proteomes of SS and AraCS cells. L. Venn diagrams showing the number of upregulated mRNAs in the transcriptome or translatome after serum-starvation for 4 days. M. Correlation between SS and AraCS cell translatomes. N. Comparison of translatomes of 3-day AraC treated cells with 9-day AraC treated cells. O-Q. Comparison of SS and AraCS cells with O. published datasets from acute lymphoblastic leukemia (ALL) stem cells and G0 fibroblast studies: with leukemia stem cells (LSC), with dormant leukemic cells (LRC), minimal residual disease (MRD) from ALL, and G0 fibroblasts. P. GSEA was performed to compare the signatures of LSC, LRC, MRD and G0 HFF with translatome of AraCS versus S+ cells, and Q. comparisons were done at multiple levels including transcriptome, translatome and proteome. Low coverage of proteome analysis is indicated with ‘N’. Data are represented as average±SEM. See also FIGS. 7A-7F.

FIGS. 2A-2M. Canonical translation is inhibited in SS and AraCS leukemic cells where global translatome and proteome analyses reveal molecular signatures specific to such G0 resistant cells. A. Mechanisms of alteration of canonical translation. B. Western analysis of translation regulators in S+ cells, and in cells serum-starved or treated with AraC for 1 day. C. Boxplot showing expression changes in the transcriptome or translatome after SS or AraC treatment, of ribosomal protein genes (left) and validated TOP mRNAs (right). D. The number of genes in the translatome and proteome, which are differentially expressed in SS and AraCS cells (fold change >1.5, p-value <0.05) compared to S+ cells. E. Gene ontology (GO) analysis of differentially expressed genes in SS and AraCS cells. Heatmap of the statistical significance of enriched G0 categories. F. Heatmap showing expression changes of transcriptome, translatome and ribosome occupancy (RO, the ratio of transcriptome to translatome) of 647 translationally regulated genes upon SS and AraC treatment. G. Gene ontology categories enriched in translationally up- or down-regulated genes shown in f. H. Heatmap of expression changes of cell adhesion genes upon SS or AraC treatment. I. Microscopic image of S+ and SS cells, which were incubated with fibronectin (FN)-coated plates for 2 hours and then washed with PBS, and the number of S+ or SS cells bound to the coated plates was calculated. J. Translationally upregulated HLA genes, HLA-E and HLA-G in SS and AraCS cells compared to S+ cells. K. Expression of immune cell modulator CD47 is increased in SS and AraCS cells compared to S+ cells at the translation and protein level in the identified translatome and proteome. L. Heatmap of expression levels of chemokines in SS and AraCS cells compared to S+ cells. M. Transwell migration assay of GFP-tagged THP1 cells and MCF7 cells that were plated in the top chamber and co-cultured with S+ or SS THP1 cells in the bottom chamber. The number of GFP-tagged THP1 or MCF7 cells that migrate through the transwell membrane to the bottom chamber is graphed. Data are represented as average±SEM. See also FIGS. 8A-8F.

FIGS. 3A-3J. Post-transcriptional and translational regulation of gene expression in G0 chemoresistant cells. A. Boxplot of the score of AU-rich elements in the 3′-UTR of genes which are up- or down-regulated in SS cells is shown. B. List of some genes having AU-rich elements (AREs) in the 3′-UTR and are up-regulated in SS cells. C.-D. Western analysis and Quantification of TNFa ARE bearing pro-inflammatory cytokine mRNA co-immunoprecipitated with FXR1 or IgG control relative to input amounts, from in vivo crosslinked extracts of S+ and SS cells. E. Scatter plot shows expression changes of RNA binding proteins upon SS and AraC treatment. TTP is indicated with a circle. F., G., H. Western analysis of TTP in S+, SS and AraC treated cells at different time points in the absence or presence of alkaline phosphatase (Pt). Phospho-TTP is indicated with arrows. I. Quantification of TNFa ARE renilla reporter expression. J. TNFα mRNA levels upon overexpression of non-regulatable, active TTP-AA or GFP in THP1 cells and in K562 cells that are then treated with AraC. Data are represented as average±SEM. See also FIGS. 9A-9F.

FIGS. 4A-4L. Pharmacological inhibition, prior to and continued with chemotherapy, of p38 MAPK and its downstream MK2 and STAT1/interferon signaling pathways—which are transiently upregulated in early G0—reduces chemoresistant cell survival. A. DDR and interferon signaling pathways activated upon serum starvation and AraC treatment. ATM signaling activates p38 MAPK that then triggers phosphorylation of MK2 and STAT1 in AraCS and SS cells. MK2 phosphorylates TTP to promote ARE mRNA levels. STAT1 activates the interferon pathway (IRDS genes) and PKR phosphorylation to phosphorylate eIF2c and inhibit canonical translation. STAT1 also increases 4EBP1 levels to inhibit canonical translation. ATM can decrease mTOR activity to inhibit canonical translation via 4EBP1 dephosphorylation. p38 MAPKα/β inhibitor, LY2228820, is shown in red. B-C. Western blotting demonstrates enhanced DDR signaling, with phosphorylation of ATM, p38 MAPK, and MK2, as well as increased STAT1-activated IFN pathway, with phosphorylation of STAT1 and PKR in SS or AraC treated cells at indicated times. D. Western blotting of phospho-STAT1 in nuclear (N) and cytoplasmic (C) extracts of AraC-treated cells at indicated times. Histone H4 was used as a nuclear maker and tubulin as a cytoplasmic marker. E. Heatmap of enhanced expression of the IFN-related DNA damage-resistance signature (IRDS) in SS or AraCS cells compared to S+ cells is shown. F. Western blotting showing reduced phosphorylation of MK2, TTP, STAT1 and PKR, and reduced levels of the ARE-bearing, TTP targeted, pro-inflammatory gene, TNFα, in AraCS cells upon treatment with 5 mM LY2228820. G. Quantification of Western analysis results shows the relative levels of phospho-p38 MAPK, phospho-MK2 and phopho-STAT1 to tubulin (loading control) in AraC-treated cells at indicated times, compared to S+ cells. H. Effect on chemoresistance with p38 MAPK inhibitor, LY2228820. THP1 cells were treated with 5 mM LY2228820 4 hours or 1 day prior to 5 mM AraC treatment (LY→AraC) or 1 day after AraC treatment (AraC→LY). Three days after drug treatment, the relative levels of cell viability and death compared to AraC treatment with vehicle control is measured using cell counting after trypan blue staining, MTS and caspase 3/7 assays. As a control for drug dosage toxicity, the effect of 5 mM LY228820 treatment alone (without AraC treatment) was compared to vehicle control in S+ cells. I. The effect of LY228820 on five AML cell lines (M5 FAB subtype) in the presence or absence of AraC treatment. Cells were treated with 5 mM LY228820 or vehicle 4 hours before AraC treatment (top) or without AraC treatment (bottom). CD34+ non-cancerous immune cells were also tested as a control. Cell viability and cell death of LY2228820-treated cells compared to vehicle-treated cells is measured by cell count, MTS and caspase 3/7 assays. J. Effect of various concentrations of BIRB on cell viability of MOLM13 cells that are untreated (S+) and treated with 1 μM AraC. K. Sequential treatment with 5 μM BirB and 1 μM AraC in MOLM13 cells. Cells were treated with 5 μM BirB 4 hours before AraC (BirB→AraC), at the same time with AraC (BirB+AraC), and 1 day after AraC (AraC→BirB). Three days after drug treatment, relative levels of cell viability and death compared to AraC treatment with vehicle control were measured. L. Western analysis of the results in J show the relative levels of phospho-p38 MAPK, phospho-MK2 and phopho-TTP to tubulin (loading control) in AraC+vehicle treated cells, AraC+BIRB treated cells, and untreated S+ cells. Data are represented as average±SEM. See also FIGS. 10A-10E.

FIGS. 5A-5L. Targeting the TNFα/NFκB pathway decreases cell survival and resistance to chemotherapy in G0 cancer cells and patient leukemic samples but not in normal, non-cancerous monocytes. A. TNFa/NFκB signaling pathway in AraCS and SS cells. TNFa pro-inflammatory cytokine is increased by p38 MAPK-MK2 pathway. Enhanced expression of TNFα, TNFα receptors, & S100A12 activate NFκB signaling, which promotes anti-apoptotic gene expression in SS and AraCS cells. Pirfenidone (PFD) inhibits TNFα and S100A12 production, and p38 MAPKγ activity. LY blocks p38 MAPKα and β. BAY11-7082 inhibits NFκB. B. Western analysis of TNFα and Tubulin (loading control) in S+, SS and AraCS cells. C. Relative expression of TNFa, TNFR1, TNFR2 and S100A12 in the translatomes of S+, SS and AraCS cells. D. GSEA showing increased expression of NFκB target genes in AraCS cells compared to S+ cells. E. Expression levels of TNFα and S100A12 in translatome of AraCS cells treated with PFD or vehicle. F. Western analysis of TNFα in AraCS cells treated with PFD or vehicle. Relative quantification shown below. G. Effect of PFD on cell survival in S+, SS and AraCS cells is shown. Cell counting, MTS and caspase 3/7 assay were performed to measure cell viability and death of THP1 cells treated with 300 mg/ml PFD or vehicle in the absence of AraC (S+, top row), in the presence of 5 mM AraC (AraCS, middle row), and with serum starvation (SS, bottom row). In the middle row, cells were treated with PFD 1 day before AraC (PFD→AraC), at the same time with AraC (PFD+AraC), and 1 day after AraC (AraC→PFD). In the bottom row, cells were treated with PFD 1 day before SS (PFD→SS), at the same time with SS (SS+PFD), and 1 day after SS (SS→PFD). H. Effect of 10 mM BAY11-7082 on cell survival in S+, SS and AraCS cells was tested as in FIG. 5G. I. Expression level of TNFα (left) and NFκB target genes (right) in the translatome of serum-starved cells across early to longer times of serum-starvation, compared to S+ cells. J. To validate the role of TNFα in cell survival upon AraC treatment, a stably transduced THP1 cell line was created with doxycycline inducible shRNA against TNFα or vector control. Cell viability and death were measured in THP1 cells with induction of shTNFa or addition of 10 ng/ml recombinant TNFa (ReTNFa) in the absence of AraC treatment (S+, top row) and in the presence of AraC (AraCS, bottom row). In the bottom row, ReTNFa is added 1 day prior to AraC treatment, and shTNFa was induced 1 day or 3 days before AraC treatment and 1 day after AraC treatment. K. Effect of PFD on six leukemic cell lines (5 M5 subtype AML, & K562 CML) in the presence or absence of AraC treatment. Cells were treated with 300 mg/ml PFD or vehicle 4 hours before AraC treatment (top) or without AraC treatment (bottom). Cell viability and death of PFD-treated cells compared to vehicle-treated cells (control) is shown in indicated cell lines. L. MOLM13 cells were pre-treated with 150 mg/ml PFD or vehicle four hours prior to treatment with various concentrations of AraC. Cell viability and death of PFD-treated cells compared to that of vehicle-treated cells (control), upon treatment with indicated AraC concentrations, is shown. Data are represented as average±SEM. See also FIGS. 11A-11H.

FIGS. 6A-6S. Co-inhibition of TNFα/NFκB, and p38 MAPK, as well as cholesterol biosynthesis-prior to and continued with chemotherapy-enhances chemosensitivity compared to individual inhibition of these resistance pathways A. GSEA reveals molecular signatures significantly enriched in the translatome and proteome of SS and AraCS compared to S+. Heatmap of normalized enrichment score (NES) is shown. Low coverage of proteome analysis is indicated with ‘N’. B. Effect of chemical inhibitors against molecular signatures (identified in 6a, and against TGFβ pathway (SB431542) and ATM (KU55933)) on cell viability of S+ and AraCS is measured by MTS assay. C. ER stress signaling pathways in SS and AraCS cells. ER stress blocks canonical translation by phosphorylation of PERK and eIF2α and increases lipid biogenesis by phosphorylation of IRE1. Cholesterol biogenesis is activated by SREBP2. Cholesterol biosynthesis inhibitor, lovastatin, is shown in red. D. Heatmap of expression levels of ER-associated genes in the transcriptome or translatome of SS and AraCS relative to S+ cells is shown. E. Western analysis shows increased phosphorylation of IRE1, PERK, and eIF2α in SS and AraCS cells at indicated times compared to S+ cells. F. Effect of lovastatin, on THP1 cell viability in the presence or absence of AraC treatment. Cells are treated with 5 μM AraC or vehicle 4 hours after various concentrations of lovastatin treatment. Cell viability and cell death of AraC-treated cells were compared with that of untreated (no AraC) cells at various concentrations of lovastatin. G. Pre-treatment of cells in five AML M5 cell lines with 6 μM lovastatin prior to treatment with 5 μM AraC, enhanced cell death, compared to vehicle pre-treatment before AraC treatment (control). H. Effect of combination therapy on chemoresistance in AML cell lines. Schematic flow of combination drug treatment with p38 MAPK/MK2, TNFα inflammation and cholesterol biosynthesis inhibitors, prior to AraC chemotherapy. Half the concentrations of the inhibitors that were previously used for individual treatments (FIG. 4G-I, 5G-H, 5K-M, 6B) were used at indicated times. MTS and caspase 3/7 assay were performed with AML cells treated with the indicated combinations of drugs in the presence of AraC treatment (AraCS, top panels) or absence of AraC (S+, bottom panels). Co-inhibition of all three pathways or any two of these three pathways, leads to enhanced sensitivity to AraC chemotherapy and decreased cell survival—without affecting S+ cells in the absence of AraC, indicating that the drug dosage used does not have a general toxic effect. I. Percentage of apoptotic and live MOLM13 cells treated with 150 μg/ml pirfenidone and 2.5 μM LY2228820, or vehicle in the presence (AraC) or absence of AraC (S+). Profiles of staining with annexin V and PI are shown. J. Quantification of colony forming units (CFU) from MOLM13 cells treated with indicated drug combinations in AraCS or S+ cells, using methylcellulose media. Microscopy shows morphology of colonies derived. K. AML cells, THP1 and MOLM13, sequentially treated with 150 μg/ml pirfenidone, 2.5 M LY2228820 and 5 μM AraC for 1 day as shown in FIG. 6H, were then analyzed for TNFα as well as phospho-MK2, and phospho-TTP levels/regulation by Western analyses. Arrows indicate the phosphorylated form of TTP. Lower panel: PL (Pirfenidone and LY2228820) combination therapy significantly enhanced phosphorylation of C-Jun N-terminal Kinase (JNK) in AraC-treated cells. L. Anti-leukemic effect of combination therapy in an MOLM13 xenograft AML mouse model. MOLM-13 cells were injected into the flanks of nod-scid mice. Mice were treated with pirfenidone (150 mg/kg, intraperitoneally) plus LY2228820 (20 mg/kg, intraperitoneally) or vehicle 1-hour prior to AraC (160 mg/kg, intraperitoneally) injection every two days for 8 days. Tumor volumes were measured at indicated time points. M. Anti-leukemic effect of PL combination therapy in a mouse model of human AML. C57BI/6 mice were intravenously injected with 5×10\hat{ }6 of HOXA9-Meis1 (AML mouse model) cells expressing luciferase respectively. IVIS imaging system (Perkin Elmer) were used to confirm engraftment of AML cells or measure leukemic burden in vivo. Mice were intraperitoneally injected with 200 ul of luciferase substrate D-Luciferin (15 mg/ml) and anesthetized. Images were taken 5 or 10 minutes after D-Luciferin injection. After confirmation of engraftment by IVIS imaging in NSG and C57BI/6 mice. Mice were randomly assigned to two group (n=3). Mice were treated with pirfenidone (150 mg/kg, intraperitoneally) plus LY2228820 (30 mg/kg, intraperitoneally) or saline vehicle 1-hour prior to AraC (30 mg/kg, intraperitoneally) treatment every day for 4 days. Leukemic burden was decreased by 58% in mice treated with combination therapy 21 days after injection of AML cells. N. Cell viability of cells from patient-derived AML samples (MGH15, MGH22, MGH25) and of non-cancerous CD14+ and CD34+ immune cells, which were treated with combination therapy (300 μg/ml PFD or vehicle, and 5 μM AraC) is shown. O. Anti-leukemic effect of PL combination therapy in primary cells from AML patients. Patient cells were pre-treated with 150 ug/ml Pirfenidone or 2.5 uM LY2228820 before treatment with 500 nM AraC. The number of live cells were measured by trypan blue exclusion test. The number of chemoresistant patient primary AML cells was decreased by 68% to 96% under treatment with PL combination therapy. P. Western blot: K562 CML cells sequentially treated with 150 g/ml pirfenidone, 2.5 μM LY2228820 and 5 μM AraC for 1 day as shown in FIG. 6H, were then analyzed for TNFα as well as phospho-MK2, and phospho-TTP regulation by Western analyses. Arrows indicate the phosphorylated form of TTP. Q. Graphs: Cell viability and TNFα mRNA levels upon overexpression of non-regulatable, active TTP-AA or GFP in THP1 cells and in K562 cells that are then treated with AraC. Shown viable cell counts and TNFα mRNA levels in THP1 and K562 cells. Right panel Western blot for myc tag showing myc tagged TTP-AA expression. R, S. Combination therapy induces apoptosis of chemoresistant leukemic cells via activation of JNK pathway. JNK has pro- or anti-apoptotic activities depending on the types of stimulus or cells. R. To assess the role of JNK in PL combination therapy-mediated apoptosis, effects of JNK inhibition were measured. A JNK inhibitor, JNK-IN-8 effectively inhibited phosphorylation of JNK and C-Jun, a direct substrate of JNK in combination therapy-treated cells. S. Furthermore, JNK inhibition partially reversed therapy-mediated apoptosis whereas it has no effect on untreated cells. See also FIGS. 12A-12E and 13. Data are represented as average±SEM.

FIGS. 7A-F. A. IC50 values of standard anti-leukemic chemotherapy, AraC, in AML cell lines. THP1 cell line was selected for this study as it shows strong resistance to AraC. B. Levels of polysome associated Ki67 mRNA in S+, SS and AraCS cells. C. Polysome profiles of S+, SS (serum starvation for 4 hours, 1 day, 2 days or 4 days), AraCS THP1 (5 μM AraC treatment for 3 days or 9 days). Heavy polysomes (>3 ribosomes) were analyzed by microarray. Polysome to monosome ratio (P/M) at indicated times of serum-starvation is shown. D. Gene ontology analysis of differentially expressed translatome across increased time of serum-starvation compared to S+. Heatmap of the statistical significance of enriched gene ontology categories. E. Correlation between translatomes of cells that were serum-starved for indicated times (i). Principal component analysis (PCA) was performed on the whole translatomes of proliferating or serum-starved cells without selection (ii). Dendrogram of the unsupervised hierarchical clustering of proliferating or serum-starved cells is represented (iii). F. Heatmap and boxplot of the expression levels of LSC gene signature (Saito et al., 2010) in transcriptome, translatome and proteome of AraCS or SS cells compared to S+ cells. Data are represented as average±SEM. See also FIGS. 1A-1Q.

FIGS. 8A-8F. A. Western analysis of p27KIP1 (p27) in S+ and SS cells as a marker for G0/G1 arrest, shows that G0 arrested cancer cells can be obtained by serum-starvation in a number of cancer cell lines. B. G0 arrest of serum-starved MCF7 is assessed by Western analysis of p27 and Hes1 levels (left) and by cell cycle analysis using BrdU and PI staining (right). C. Polysome profiles of S+, SS cells from MCF7, U2OS, HepG2 and non-cancerous HFF fibroblasts cell lines. Heavy polysomes (≥3 ribosomes) were analyzed by microarray. D. Expression levels of up- or down-regulated translatome from FIG. 2A in the translatomes of SS G0 MCF7, HFF, HEPG2 or U2OS compared to S+ cells. E. GSEA reveals molecular signatures enriched in the translatome or proteome of SS or AraCS cells from five different cell lines. Heatmap of normalized enrichment score (NES) is shown. Low coverage of proteome analysis is indicated by ‘N’. F. PCA analysis (i) and unbiased hierarchical clustering (ii) of the whole translatomes of SS or AraCS cells from five different cell lines are shown. Data are represented as average±SEM. See also FIGS. 2A-2M.

FIGS. 9A-9F. A. mRNAs having increased RO have structured RNA in their 5′-UTRs relative to mRNAs having decreased RO. The average lengths of the 5′-UTRs are similar between these two groups. B. GSEA showing down-regulation of genes involved in the decay of ARE bearing mRNAs in SS THP1 (top) or SS MCF7 (bottom) compared to S+ cells. C. Heatmap of expression changes of exosome complex (3′-5′ exonuclease RNA decay and processing complex) genes upon serum starvation in indicated cell lines. D. Boxplot showing reduced expression of proteasome complex genes upon serum starvation and AraC treatment. E. Heatmap of expression changes of ARE binding proteins upon SS, AraC and PFD treatments is shown. These proteins are known to cause ARE mRNA decay or translation repression. F. AU-rich element score in the 3′-UTRs of mRNAs that are up- or down-regulated upon FXR1 depletion (16). Data are represented as average±SEM. See also FIG. 3A-3H.

FIGS. 10A-10E. A. Western analysis of proteins associated with DDR (phospho-p38 MAPK, phospho-MK2), interferon response (phospho-STAT1), and ER stress (spliced XBP1) in S+ cells and in cells treated by serum-starvation or with AraC for indicated times. B. Box plot showing the expression level of IRDS genes in SS and AraCS cells compared to S+ cells is shown. C. Western analysis of enhanced phosphorylation levels of STAT1 in SS cells, which is reduced upon treatment with 5 μM LY2228820. D. MV4:11AML cells were pre-treated with 5 μM LY2228820 or vehicle four hours before treatment with various concentrations of AraC. Cell viability and death of LY2228820-treated cells compared to that of vehicle-treated cells (control) is shown after treatment with indicated AraC concentrations. E. Cell proliferation assay was performed with BrdU incorporation in MV4: 11 and MOLM13 AML cell lines. Percentage of BrdU-positive AraCS cells is shown after treatment with 5 μM LY2228820 or vehicle. Data are represented as average±SEM. See also FIGS. 4A-4L.

FIGS. 11A-11H. A. Heatmap and boxplot of the expression level of senescence-associated secretory phenotype (SASP) genes in the transcriptomes, translatomes, and proteomes of AraCS or SS compared to S+ are shown. B. GSEA showing increased NFκB target genes in the translatome of SS cells compared to S+ cells. C. Relative translatome levels of BCL2A1, BCL3 and BCL6 in cells serum-starved for indicated times. D. Cells were pre-treated with 300 μg/ml PFD or vehicle for various times (0-48 hours) prior to 5 μM AraC treatment. Cell viability and cell death of PFD-treated cells are shown relative to vehicle-treated cells. E. Cell cycle analysis of THP1 cells treated with recombinant TNFα or vehicle for 1 day, using BrdU incorporation and PI staining followed by flow cytometry. F. THP1 cells with constitutive expression of shRNA against S100A12 or control shRNA were treated with various concentrations of AraC (0 μM, 5 μM and 10 μM), followed by MTS and caspase assay to measure cell viability and cell death. G. MCF7 cells were pre-treated with 300 μg/ml PFD or vehicle 1 day before treatment with serum starvation or 100 nM doxorubicin. Cell viability of PFD-treated compared to vehicle-treated cells is shown. H. Combined effect of PFD and shRNA against TNFα on chemotherapy survival. Cell viability and cell death assay were performed with THP1 cells in which the shRNA against TNFα or control shRNA was induced 3 days before AraC treatment, with or without 300 μg/ml of PFD added 1 day before AraC treatment. Data are represented as average±SEM. See also FIGS. 5A-5L.

FIGS. 12A-E. A. Table showing concentrations, biological targets and status of chemicals used. B. Effect of chemical inhibitors against molecular signatures (identified in a.) on cell viability of SS cells is measured by MTS assay. C. Multiple AML M5 cell lines were treated with 6 μM lovastatin or vehicle in the absence of AraC. Cell viability and cell death assays of treated cells compared to vehicle-treated cells (control lane) are shown. D. Schematic flow of treatment of inhibitors against ATM (KU55933), TNFα inflammation (pirfenidone), and cholesterol biosynthesis (lovastatin) prior to treatment with AraC chemotherapy in THP1 cells. Cell count, MTS and caspase 3/7 assay were performed after treatment with this combination therapy. E. Anti-leukemic effect of PL (Pirfenidone and LY2228820) combination therapy in mice engrafted with human AML cells. MOLM13 xenograft AML mouse model was created by injecting MOLM-13 cells into the flanks of nod-scid mice. Mice were treated with pirfenidone (150 mg/kg, intraperitoneally) plus LY2228820 (20 mg/kg, intraperitoneally) or vehicle 1-hour prior to AraC (160 mg/kg, intraperitoneally) injection every two days for 8 days. Tumor volumes were measured at indicated time points. The leukemic burden was decreased by 77% in mice treated with combination therapy. Data are represented as average±SEM. See also FIGS. 6A-6S

FIG. 13. Hypothetical Model. Post-transcriptional and translation regulation of gene expression that leads to chemoresistance and G0 cell survival is regulated by DNA damage and stress signaling that is triggered in subpopulations of cancer by genomic instability and stress, as well as induced by chemotherapy and serum-starvation.

### DETAILED DESCRIPTION

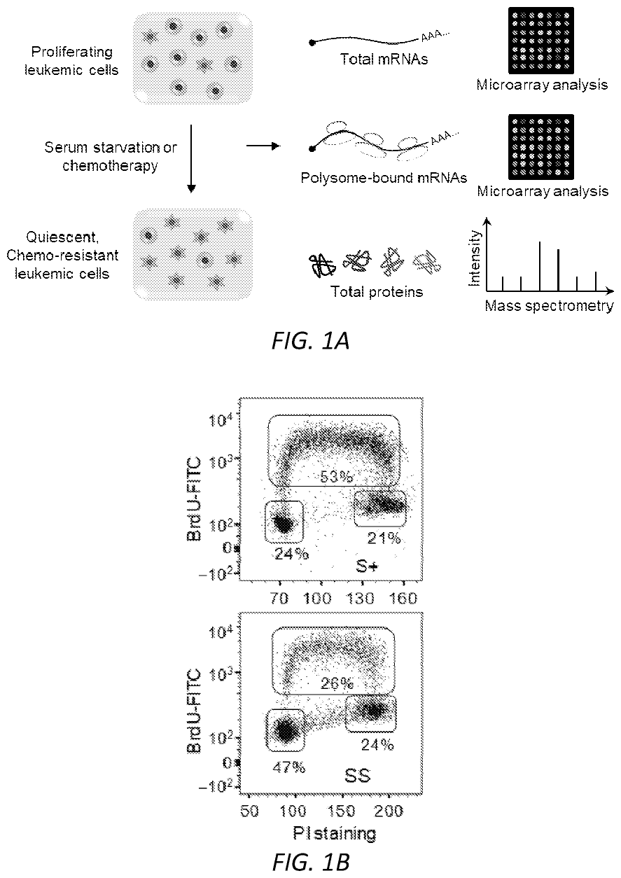

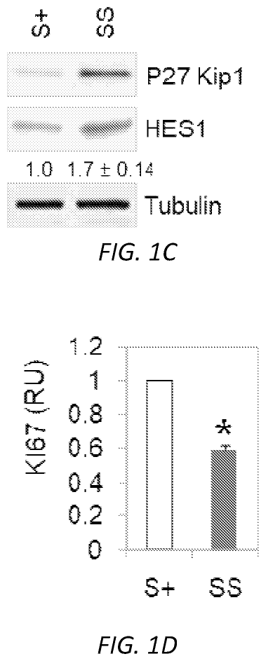

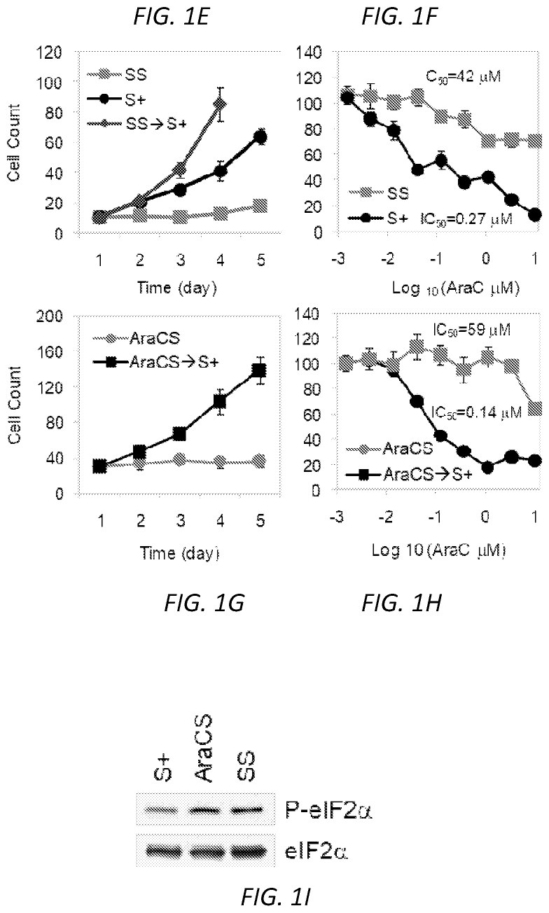

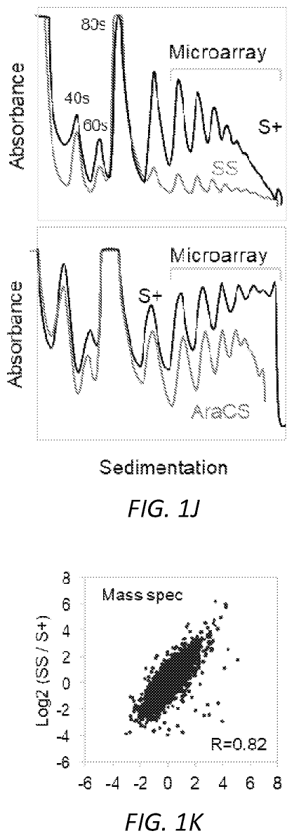

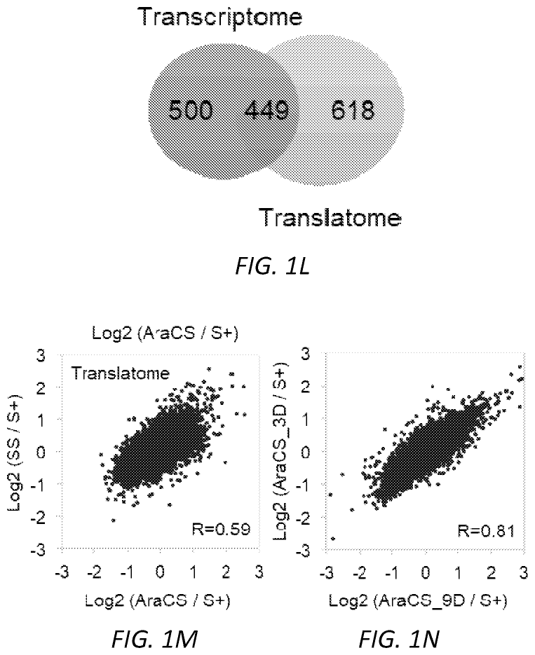

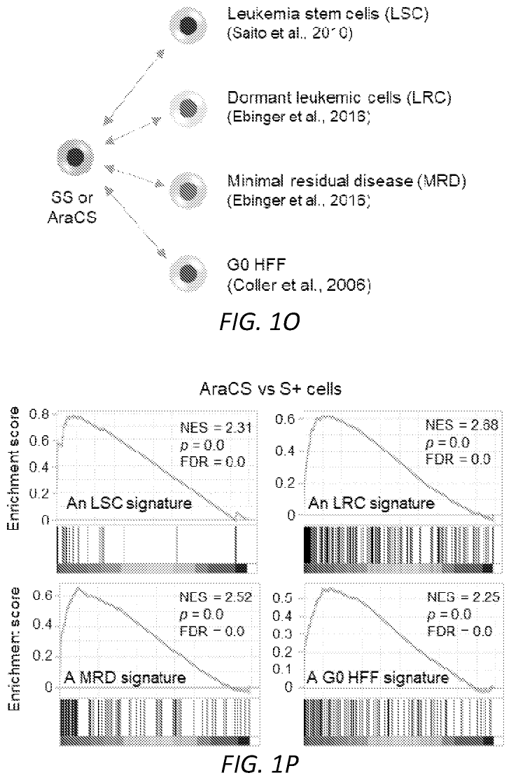

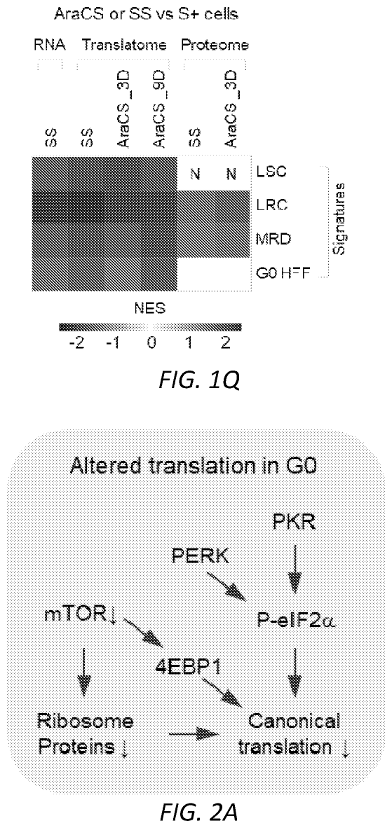

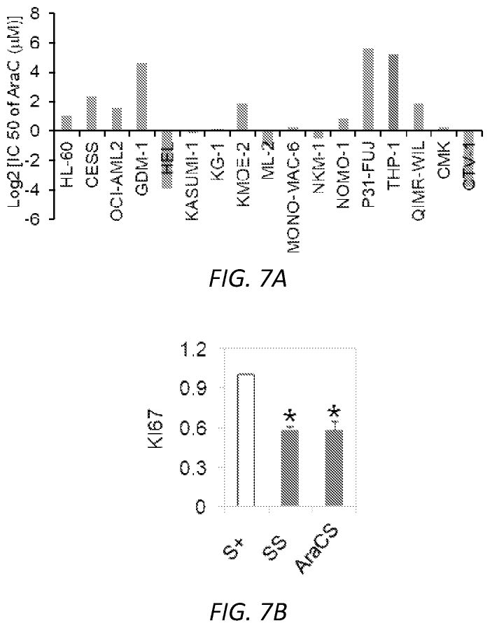

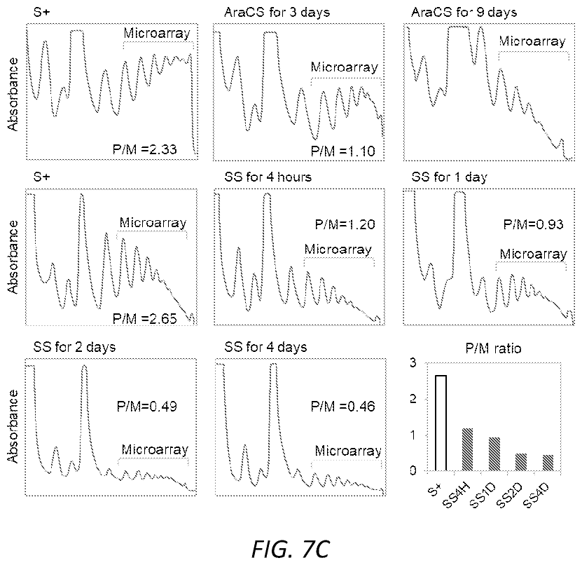

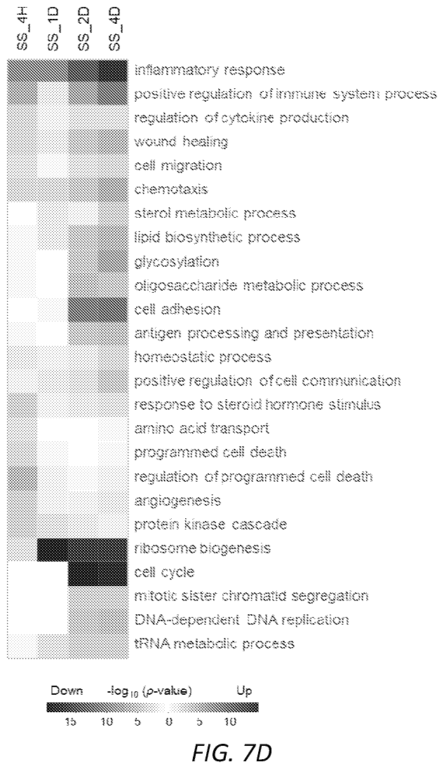

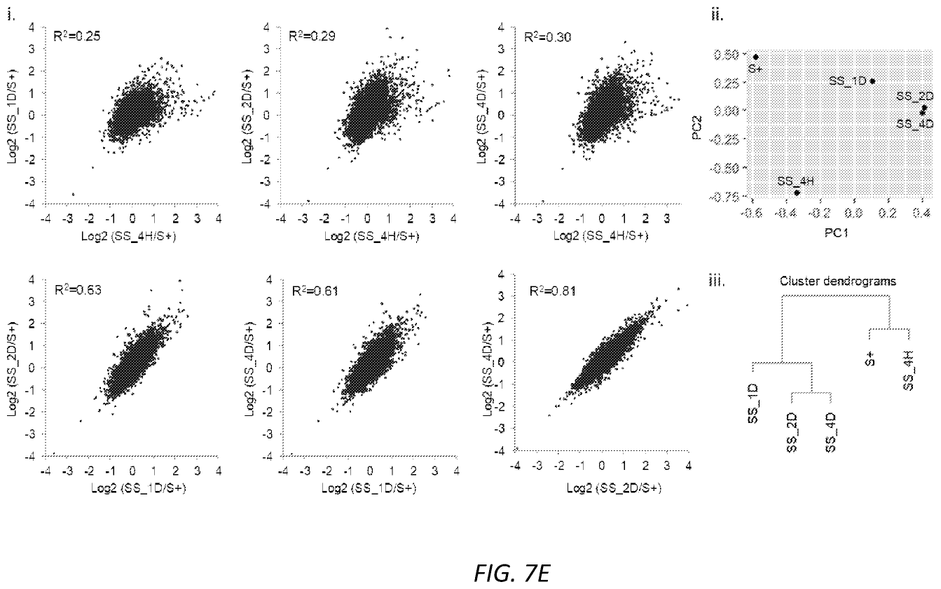

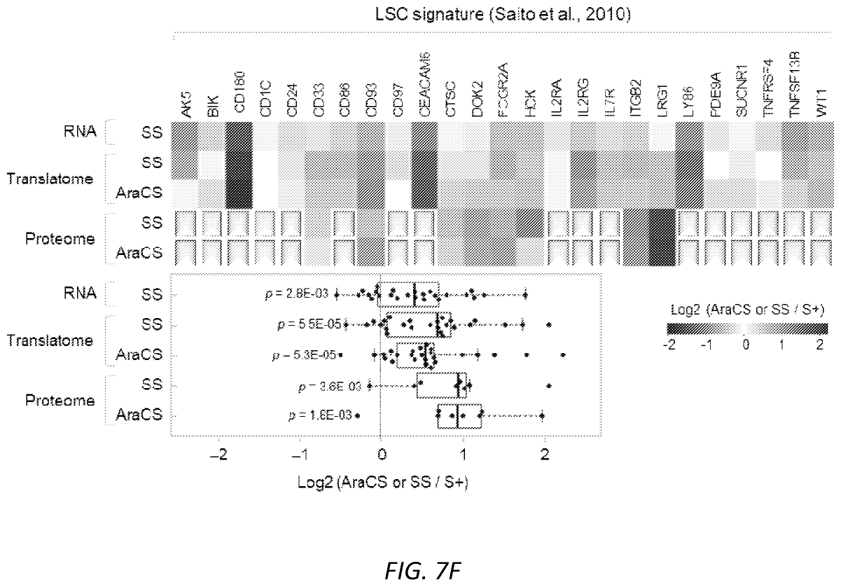

G0 cells are a transiently arrested, clinically relevant subpopulation in cancers (4-7, 12, 13, 15-26). Previous work revealed altered gene expression mechanisms in G0 leukemic cells; in particular, at the post-transcriptional (16, 28-30, 157) and translational level (31-33). This would lead to an altered gene expression profile that could underlie G0 cell survival in harsh conditions. G0 cells are resistant to harsh conditions like serum-starvation, with transient inhibition of apoptosis and proliferation (3, 11, 27, 33), which are features required for cells to survive chemotherapy. Importantly, SS cells—G0 leukemic cells induced by growth factor deprivation—exhibit chemoresistance (FIG. 1F); consistently, true chemo-surviving AraCS cells are transiently arrested (FIGS. 1G, 7B). As shown herein, SS cells were similar in translatome and proteome to AraCS chemosurviving cells (FIG. 1K-N), indicating their similarity. Published signature genes of G0 fibroblasts and of leukemic stem cells that are chemoresistant (3-7, 16, 27, 34-36) were also highly expressed in SS and AraCS cells (FIGS. 1O-1Q, 7F). Thus, the common G0 resistance gene expression profile observed in AraCS chemo-surviving cells and SS G0 cells-distinct from neighboring proliferating tumor cells-likely comprised genes that control survival and resistance. These data revealed that in addition to known transcriptional changes (3-7, 16, 27, 34-36), altered post-transcriptional and translation mechanisms in G0 cancer cells contribute in part, to their unique gene expression profile that underlies their chemoresistance.

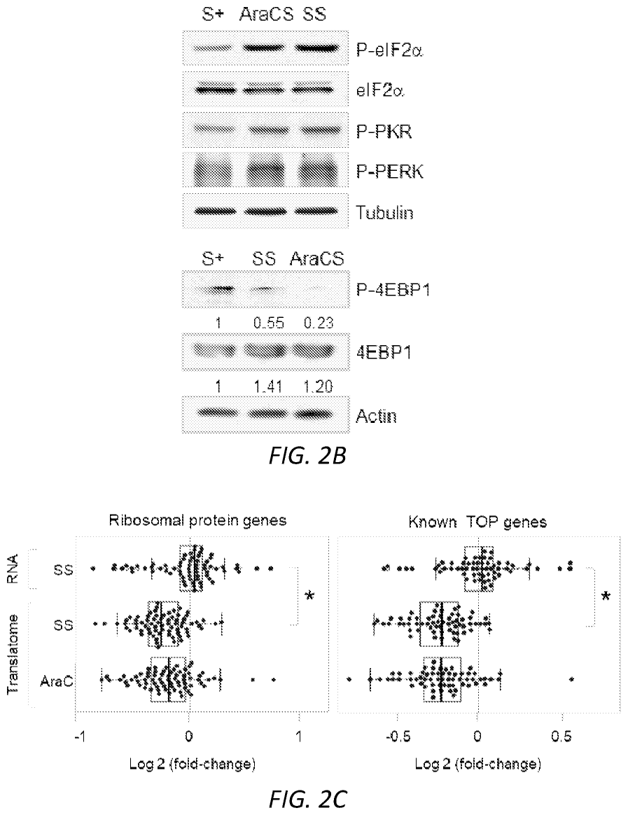

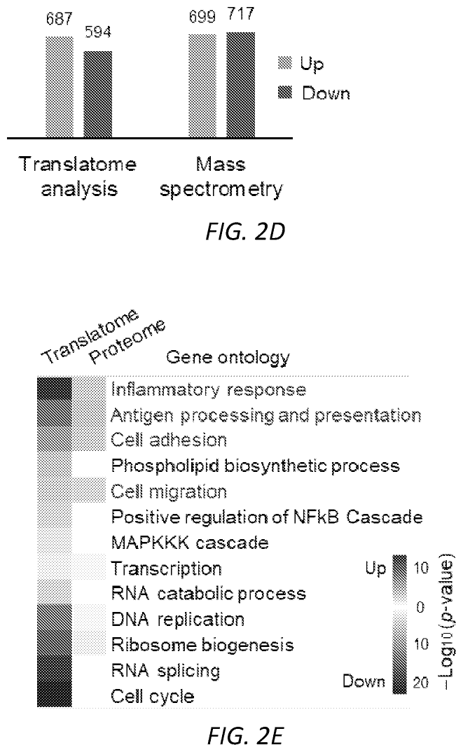

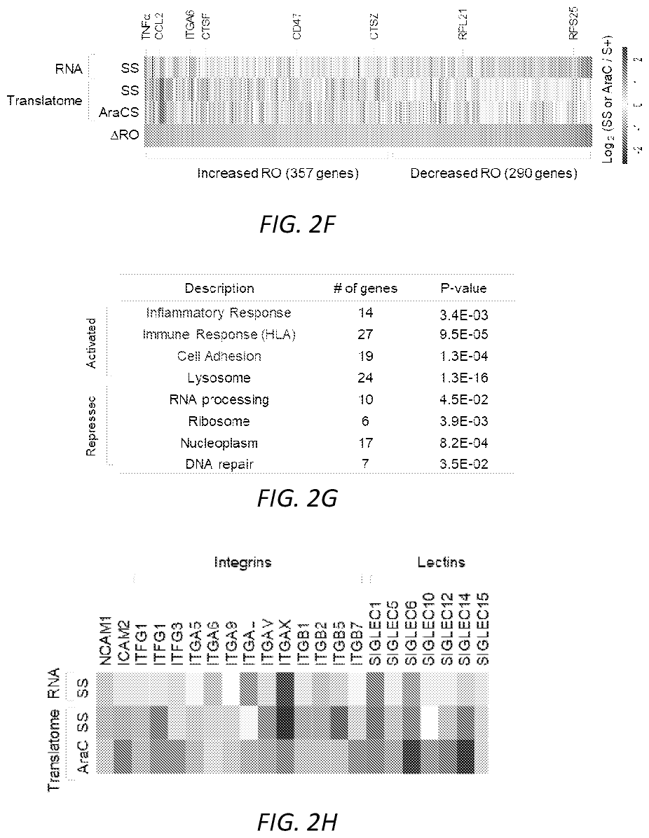

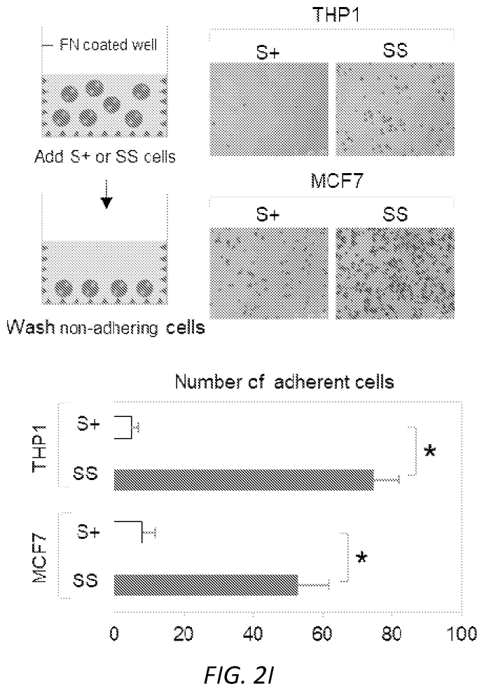

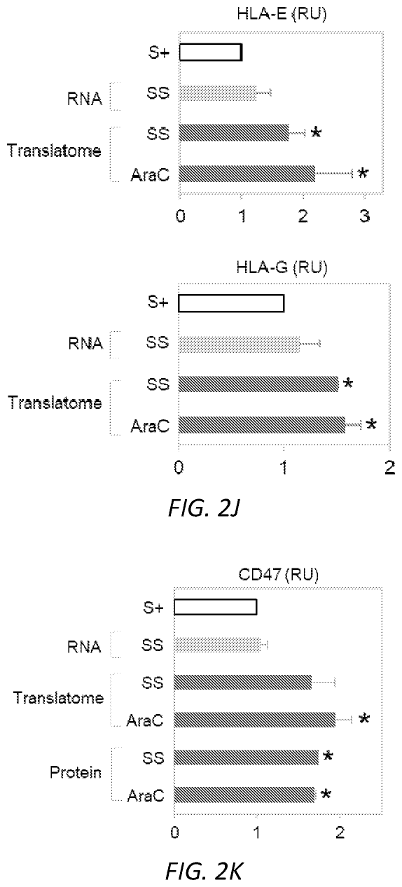

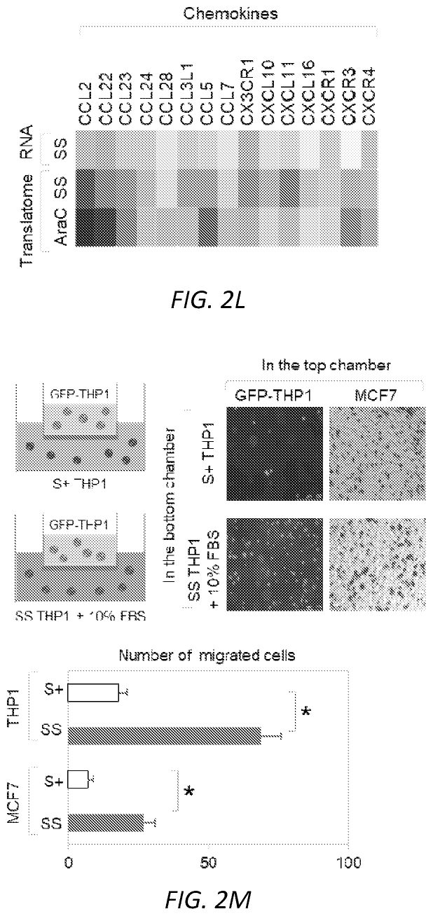

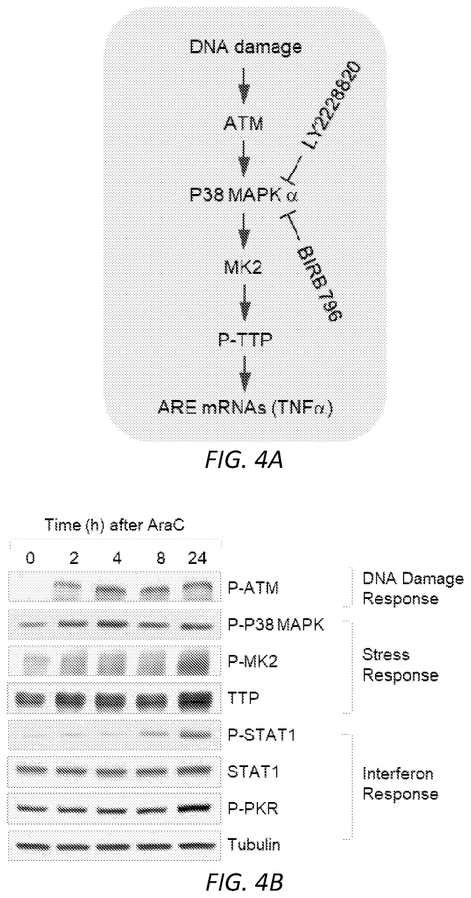

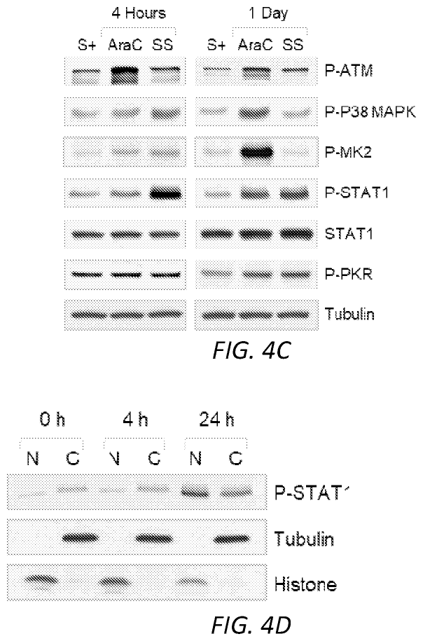

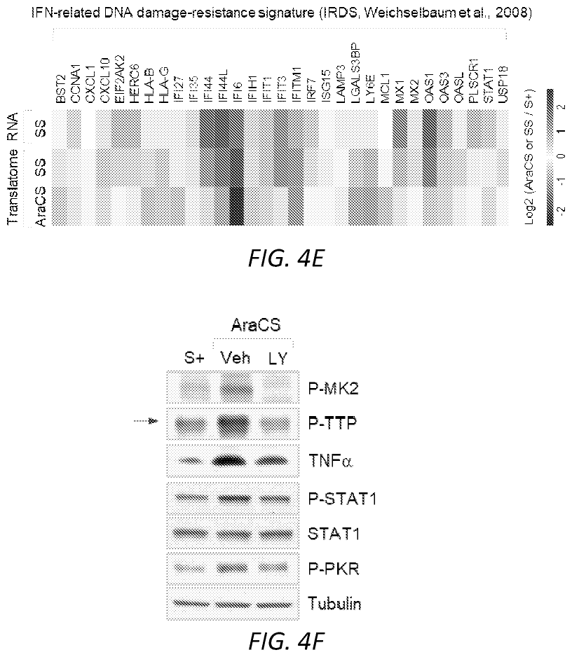

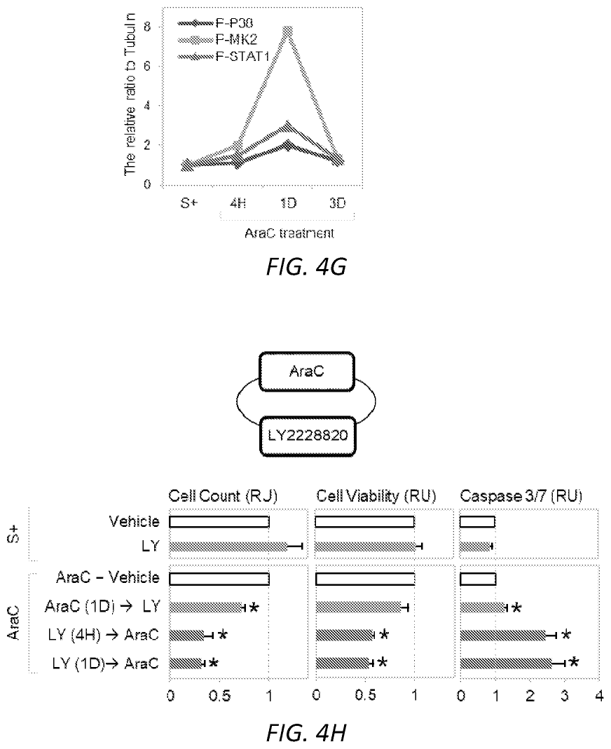

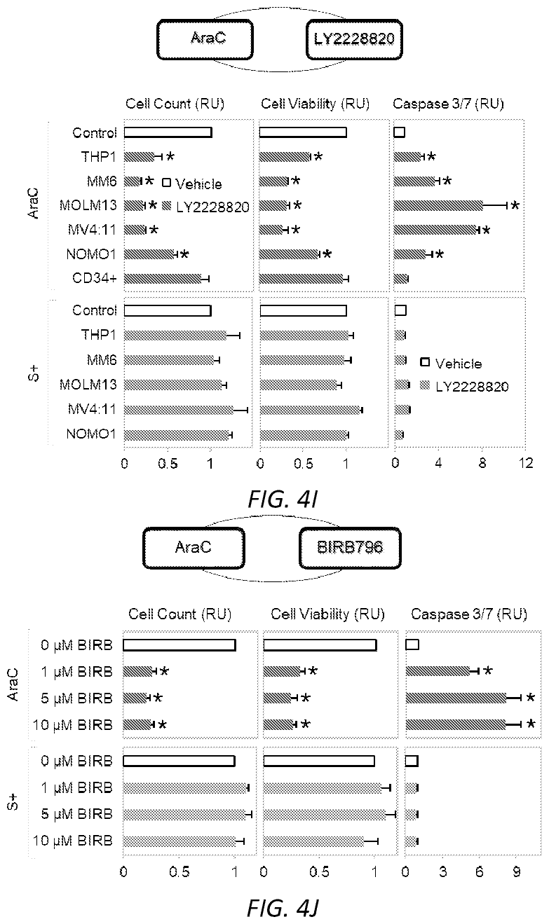

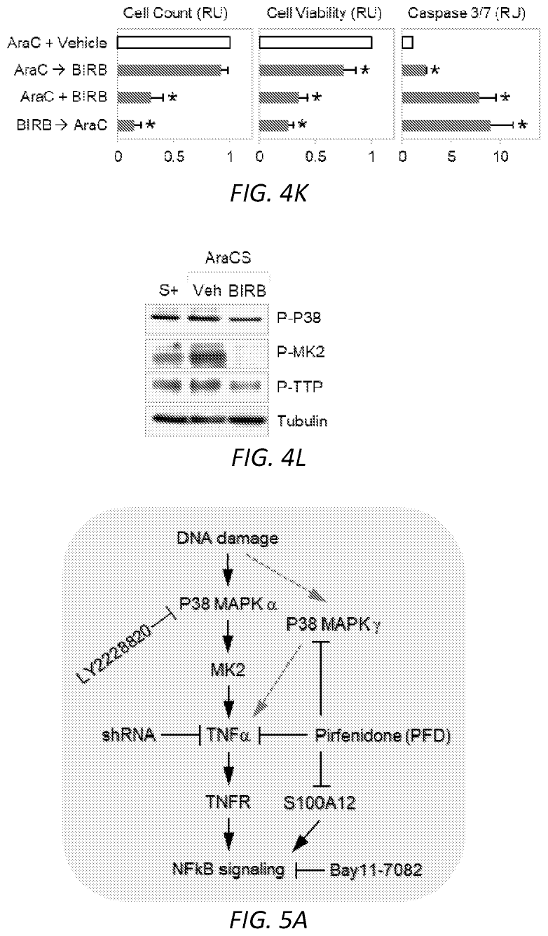

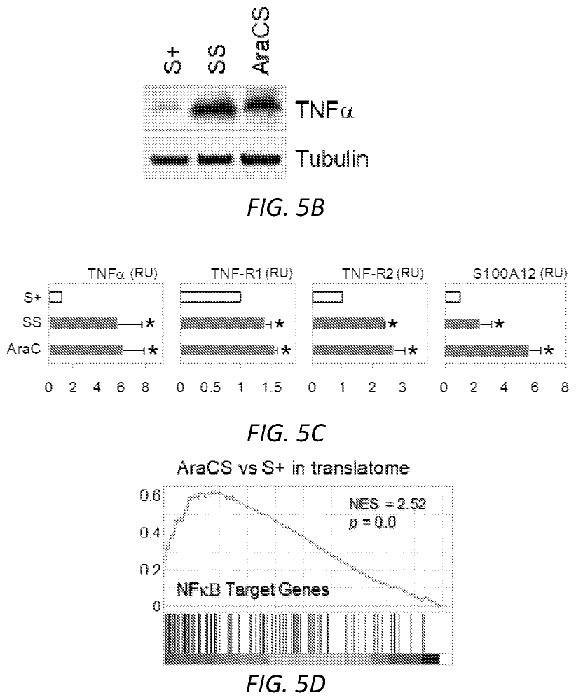

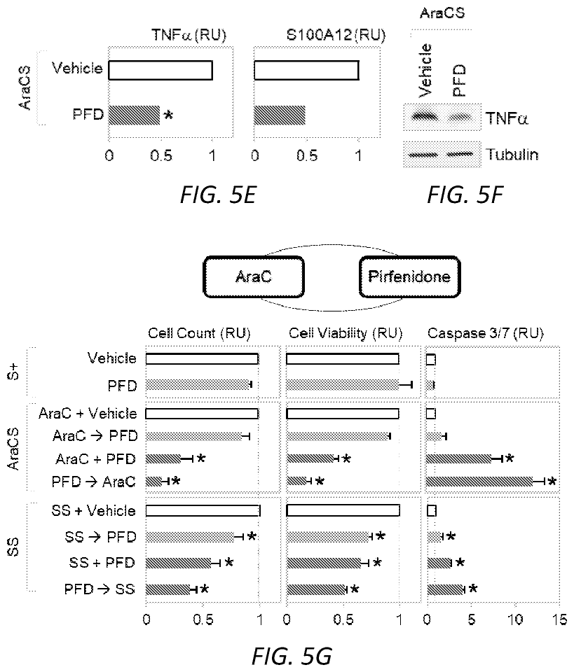

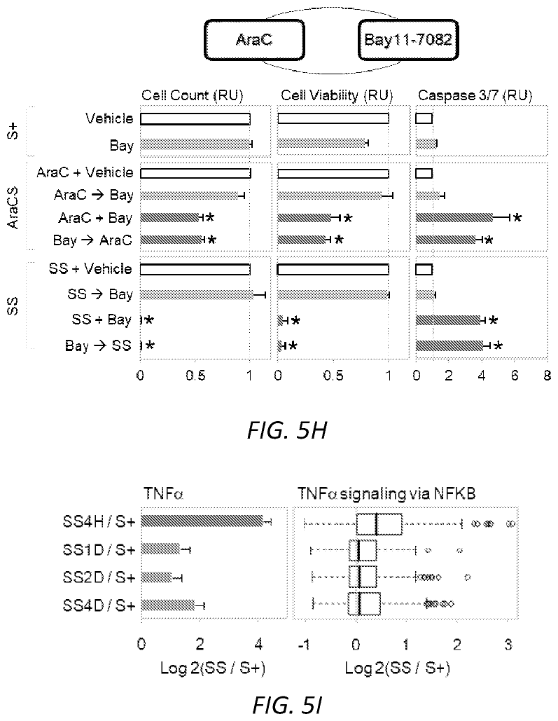

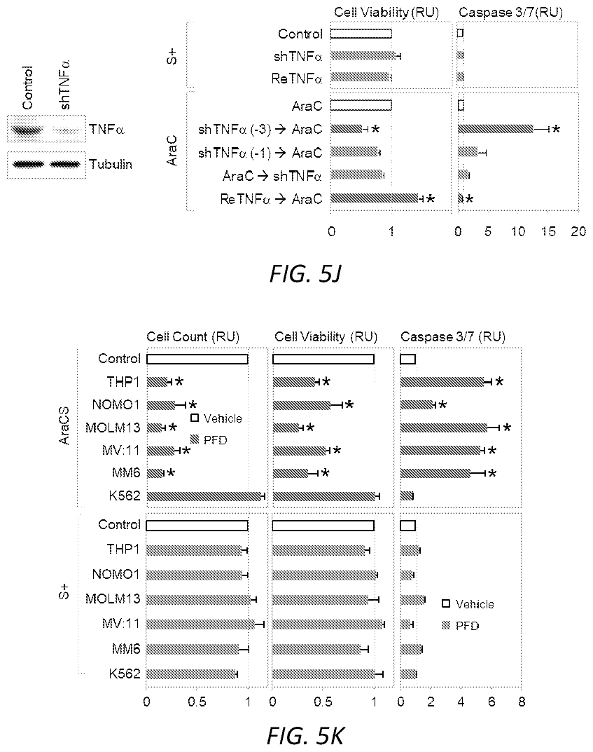

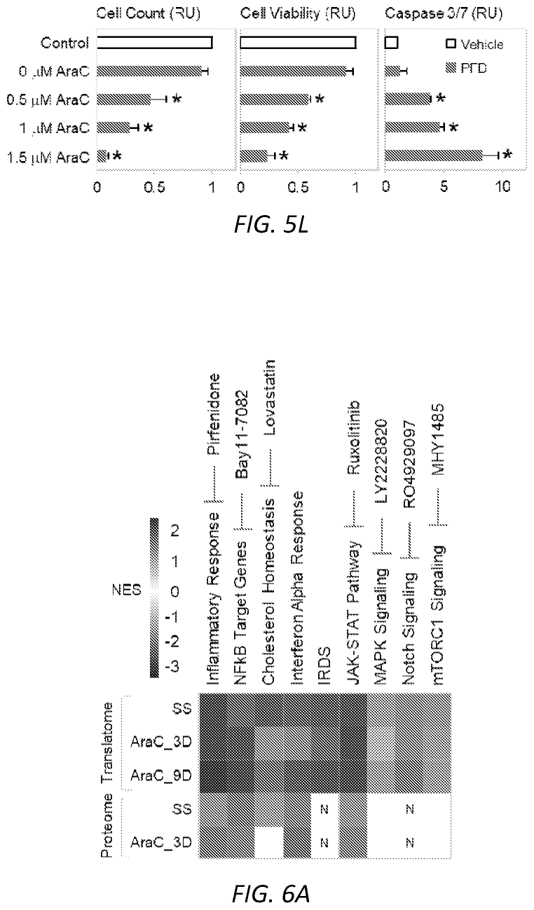

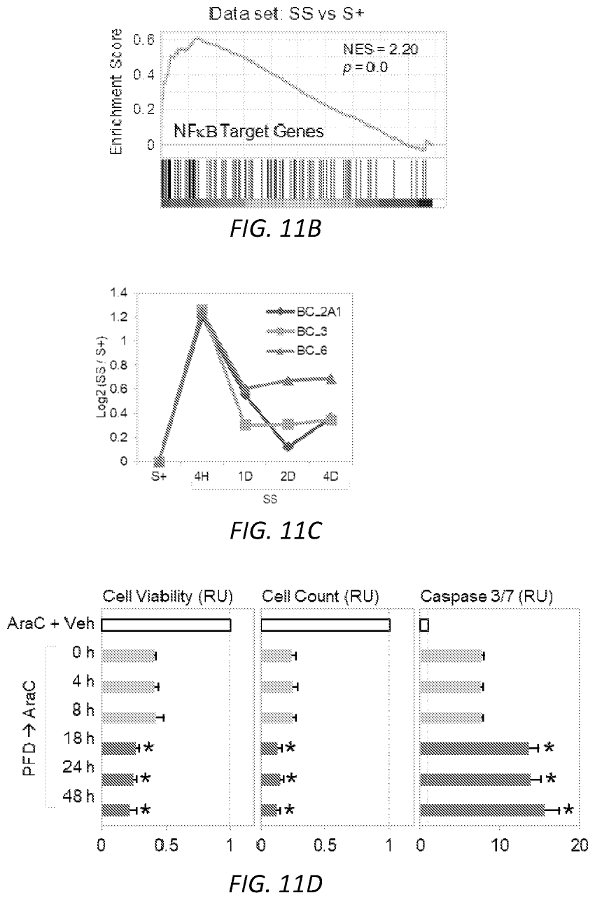

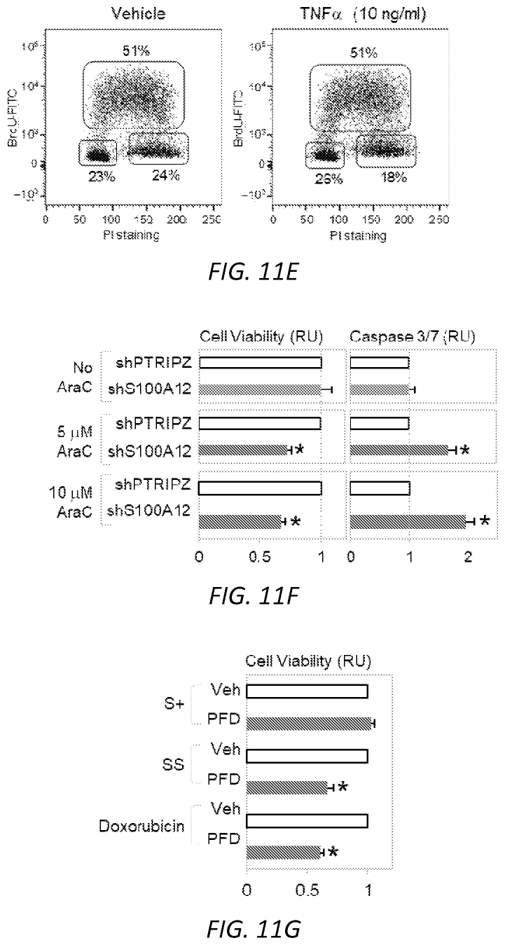

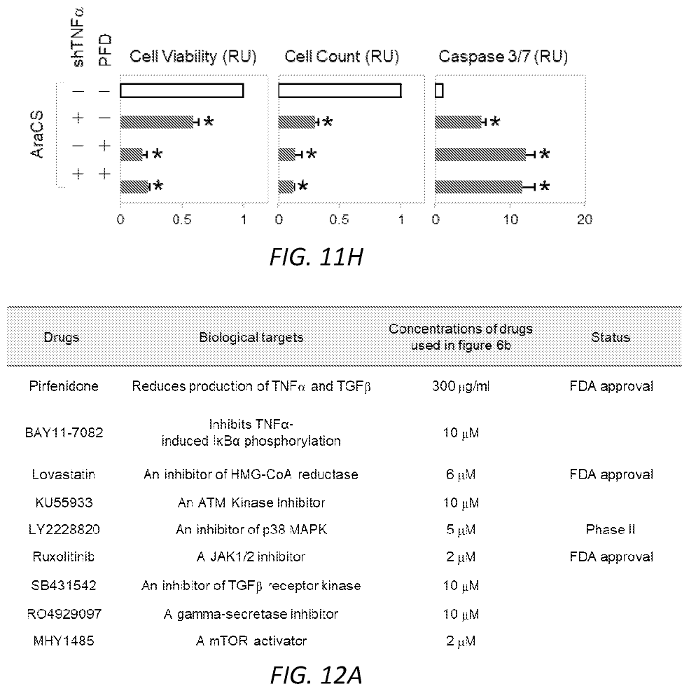

These findings revealed the importance of DNA damage and stress signaling that can initiate an inflammatory response that leads to survival and resistance instead of cytotoxicity (FIGS. 4-6, 10-12). Differential genomic instability in cancers would lead to distinct subpopulations within a tumor with disparate DNA damage response and stress signaling that enables their survival (40-42). We found that DDR mediated ATM signaling induced inflammatory/immune effectors that can mediate chemotherapy survival. These include interferon response (82-86) (FIG. 4A-4I), and inflammatory cytokines (FIG. 2E-2G, 5B-5C) that promote downstream NFκB activated pro-survival target genes (245-249) including BCL anti-apoptotic genes (250, 252, 253) (FIGS. 5A, 5D, 11B-11C). Treatment with reagents against these resistance-enabling immune regulators after chemotherapy is not very effective as the downstream survival effectors have already been induced, and targeting their upstream cytokine regulators would not be effective (FIGS. 4F-4I, 5G-5H, 5K-5M, 11D, 6H). Therefore, treatment with reagents that block these resistance pathways prior to (and continued with) or along with chemotherapy treatment, enables the most effective outcomes, as they prevent further enrichment and establishment of such cells by blocking induction of pro-survival signaling.

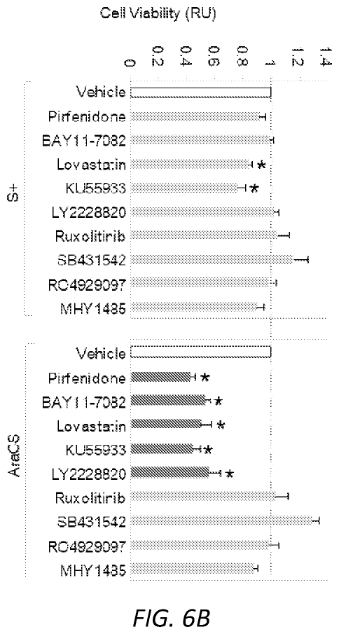

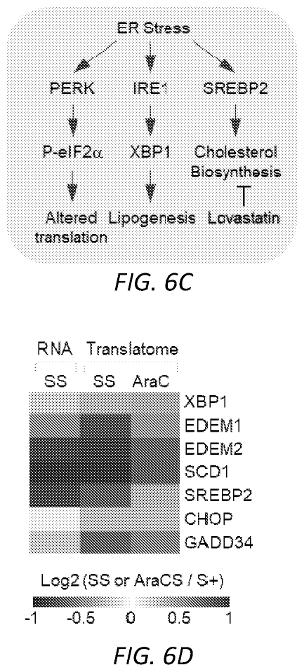

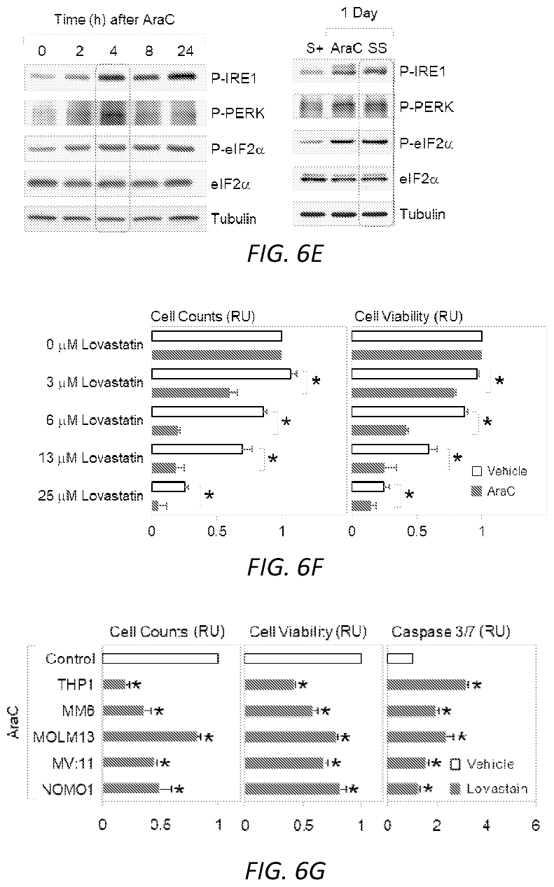

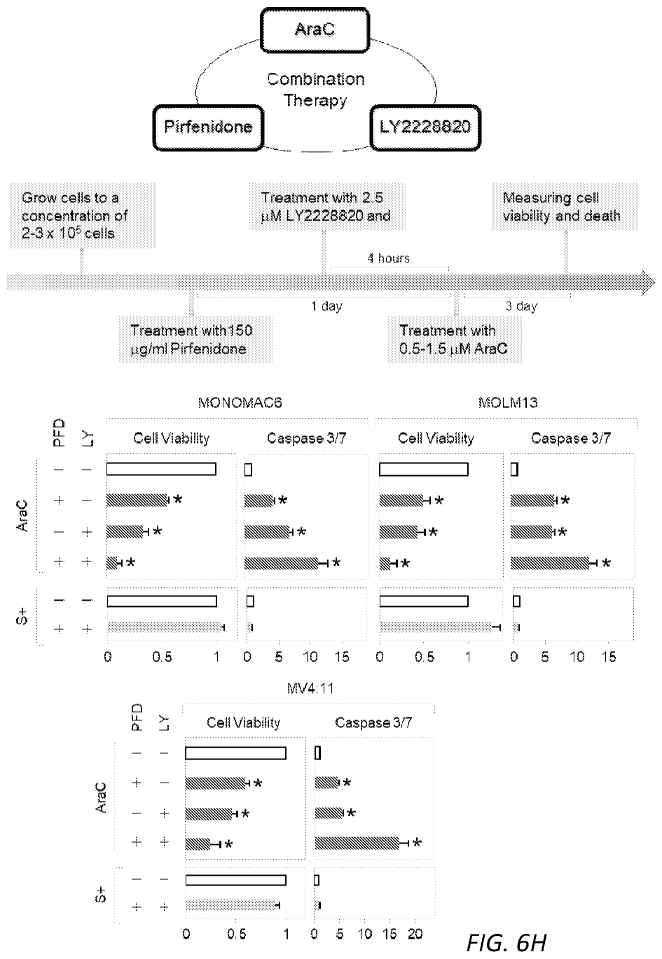

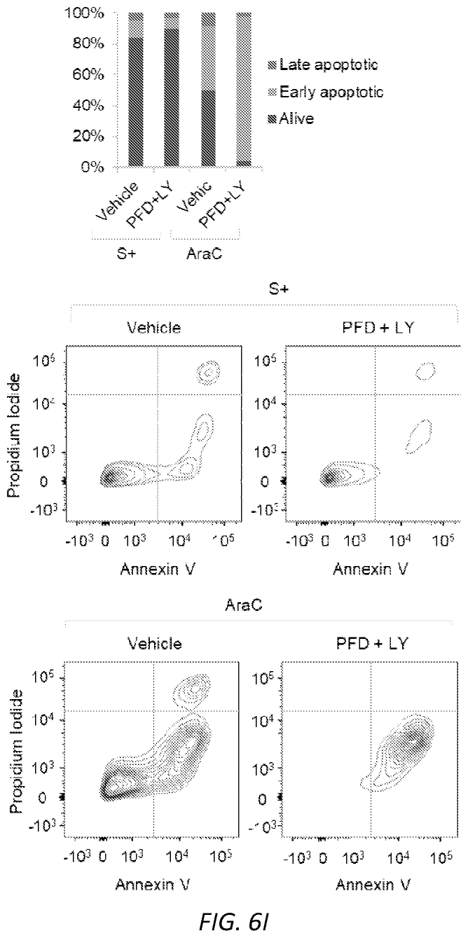

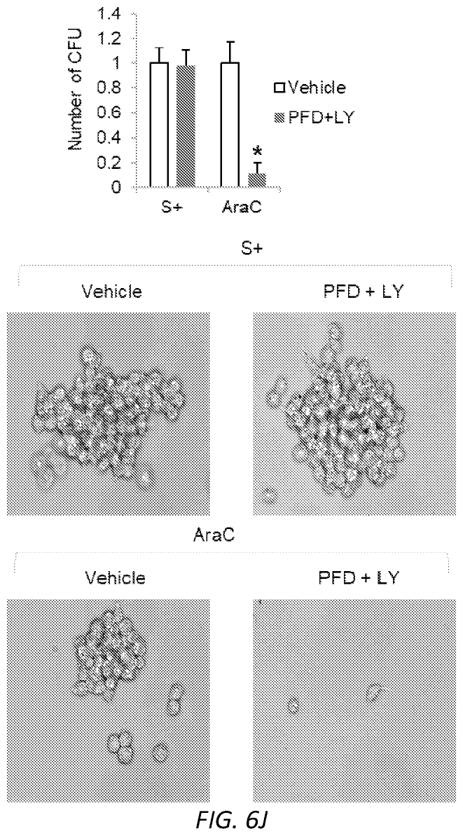

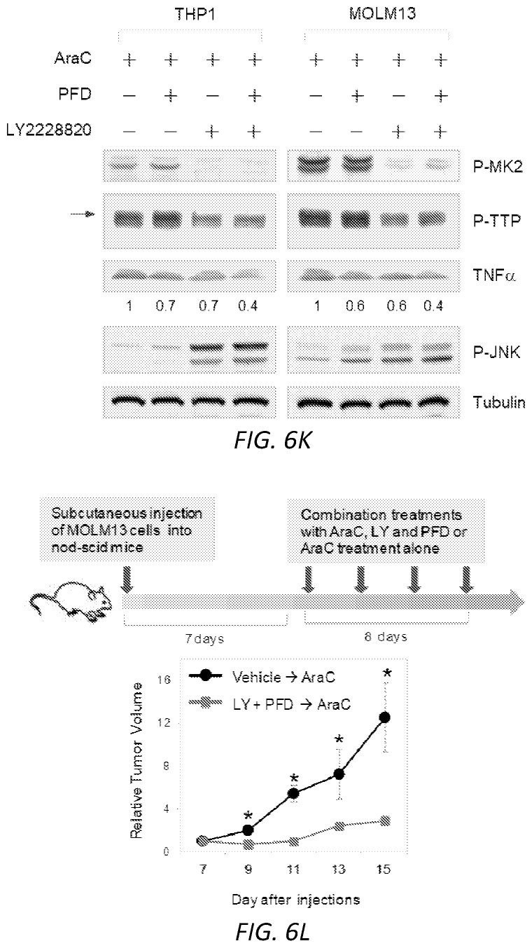

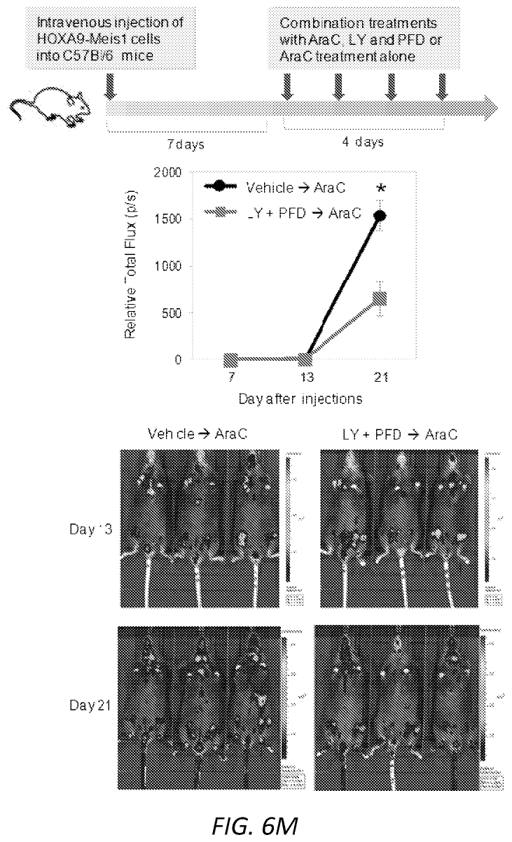

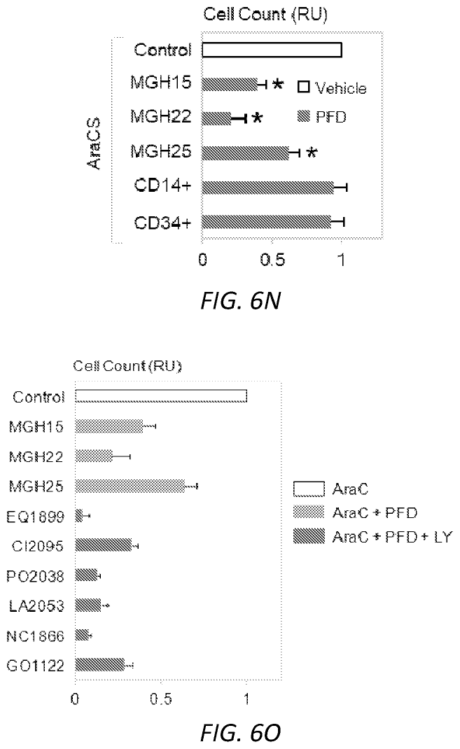

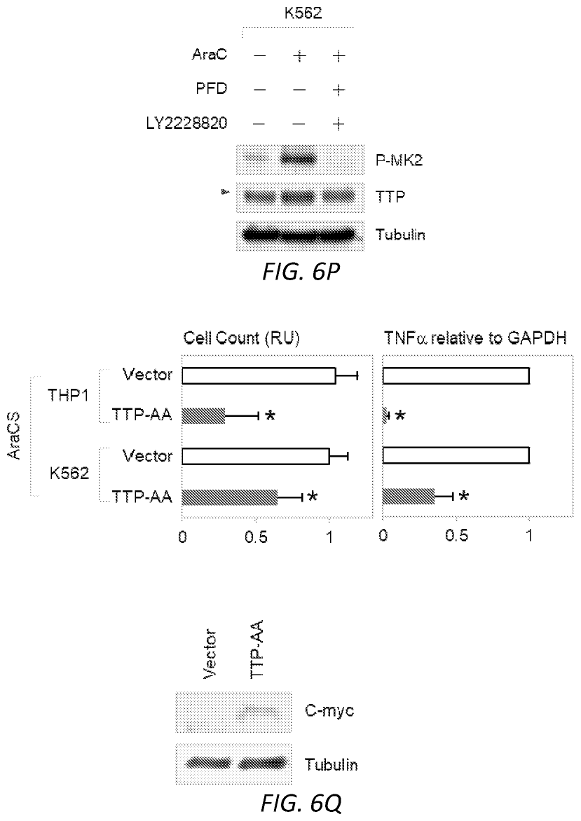

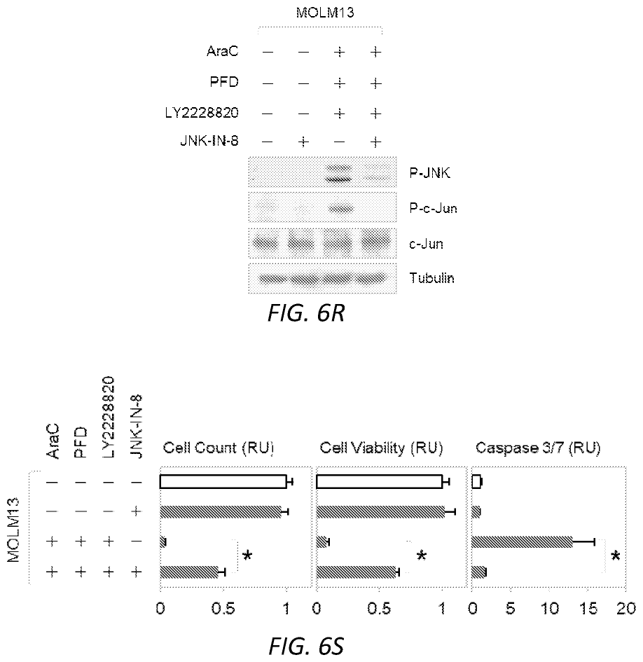

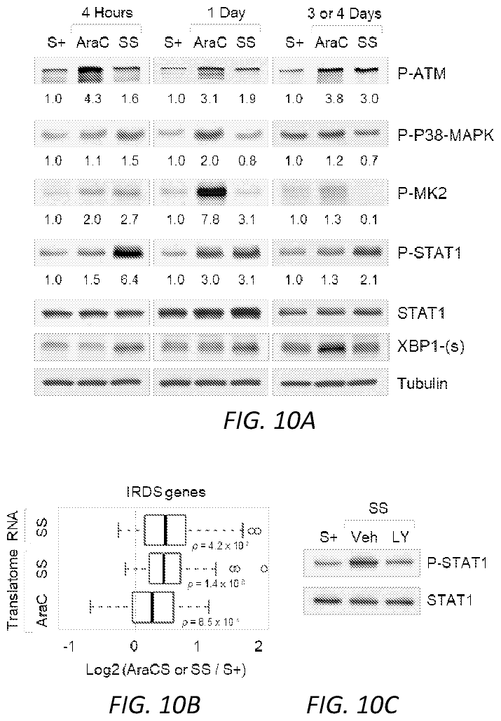

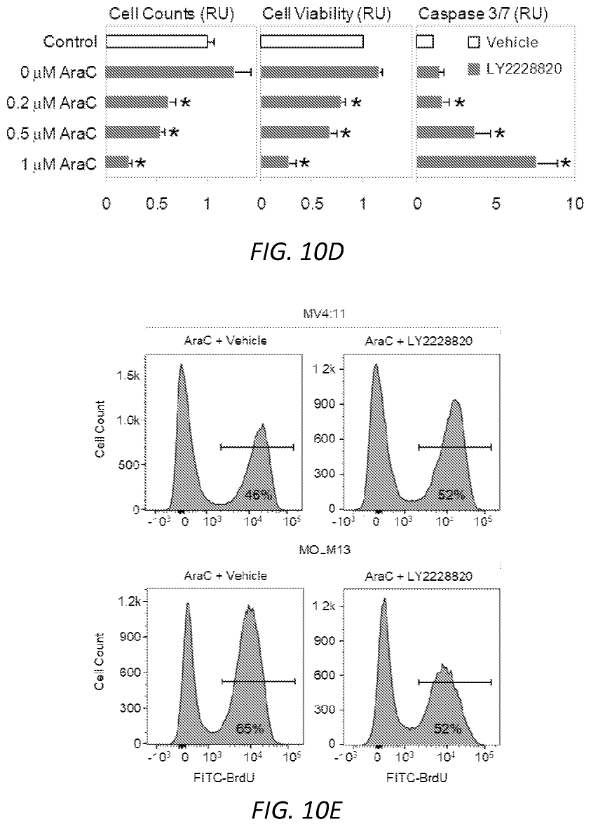

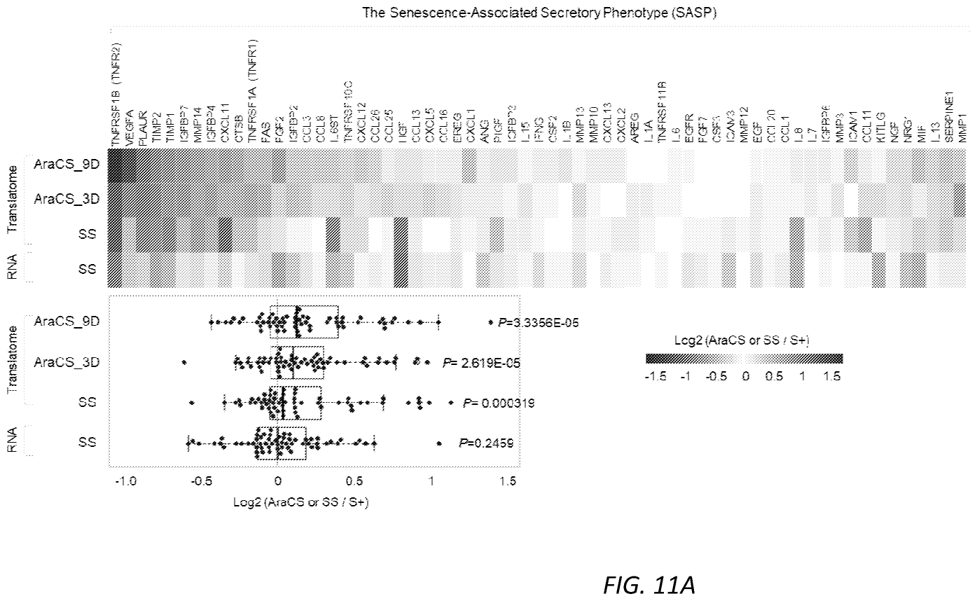

AraC is a nucleotide analog and replication inhibitor and therefore, triggers DNA damage signaling (103-105). Increasing the concentration of AraC would cause further DNA damage signaling (40-42) and should lead to more cells expressing this inflammatory pathway that enables their resistance—and thus alter more cells to enter the inflammatory active phase that can be targeted by inflammation inhibitors. Consistently, we found that increased AraC treatment of THP1 cells lead to more cells entering the inflammatory phase that is targeted by pirfenidone and lead to further decrease in resistance (FIG. 5L). Pertinently, we observed the above survival triggered by other DNA-damage inducing drugs such as doxorubicin (258, 259) in other cancers such as SS G0 MCF7 breast cancer cells, which is similarly reduced by the stress and inflammation inhibitor, pirfenidone (FIG. 11G). These data suggest that certain chemotherapies and stresses like serum-starvation induce DNA damage and stress signaling (FIGS. 4A-4C, 10A) and promote enrichment and establishment of resistant G0 cells—in addition to pre-existing subpopulations with genomic instability that could trigger DNA damage and stress signaling (40-42). These effects were observed in many AML and breast cancer cell lines but not all cancers (FIG. 5K), indicating specificity in G0 expression and resistance. Importantly, these resistance pathways could be blocked to significantly decrease resistance to AraC treatment, not only in different AML cell lines (FIG. 6H) but also in tumors in vivo in an AML xenograft mouse model (FIG. 6L) as well as in multiple patient-derived AML samples—without affecting normal cells (FIG. 6M)—supporting their potential applicability against resistance and cancer persistence in AML.

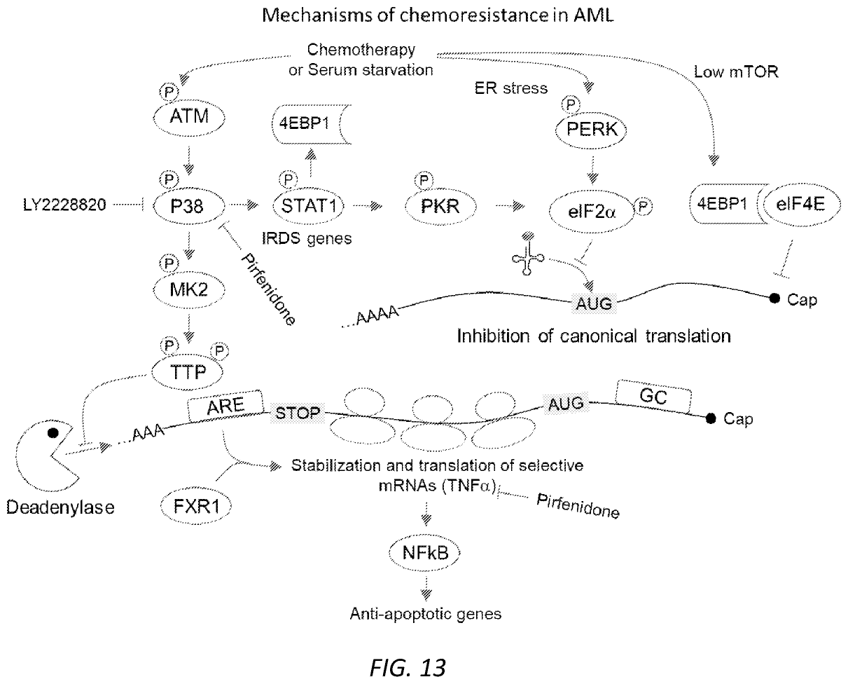

We found three key downstream resistance pathways (FIG. 13), mediated by AraCS and SS treatments (FIGS. 4A, 5A, 6B-6C, 12C), that altered post-transcriptional and translational gene expression and enable resistance. These include:

1. DNA damage ATM (40-42) and stress activated p38 MAPK signaling (44-49) that in turn promoted several downstream survival effectors (FIGS. 4, 10): a. p38 MAPK activated MK2 (64-66) post-transcriptionally upregulated ARE bearing mRNAs (67-73) (FIGS. 2E-2G, 3B-3C, 3H, 4A-4F, 5A-5C, 5F-5M, 11B-11F), including cytokines, immune modulators (HLA-E, HLA-G (73, 87-90), CD47 (91-96), FIG. 2J-2K) that are known to promote resistance (97), as well as chemokines that induce nearby cell migration (FIG. 2L-M), b. p38 MAPK activated STAT1/interferon pathway enabled upregulation of IRDS genes that are associated with resistance (82-86) (FIGS. 4A, 4E, 4F-4I), and increased immune modulators (FIG. 4A, 4F, 10A-10C, 5A-5F), and inhibited canonical translation via PKR activation of eIF2α phosphorylation as well as via increasing 4EBP (FIGS. 2A-2B, 4B-4C, 4F);

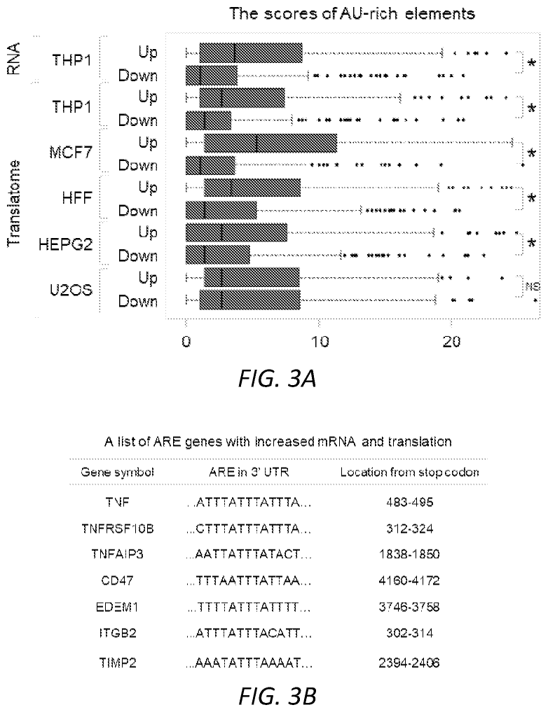

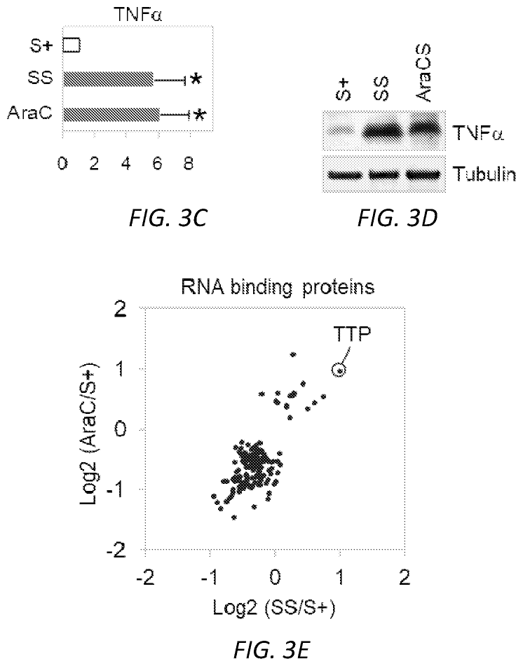

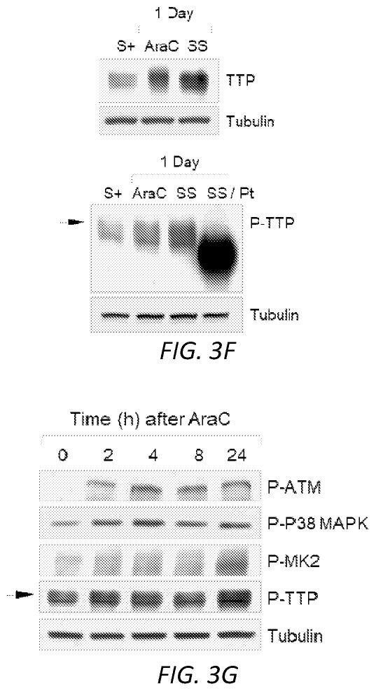

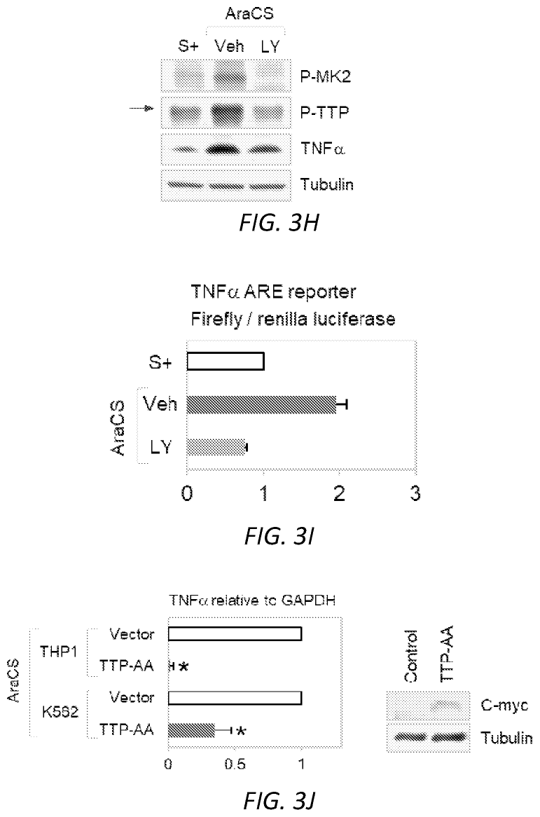

2. ATM mediated suppression of mTOR activity (40-42, 226) to inhibit canonical translation via 4EBP dephosphorylation that enabled non-canonical translation (32) (FIGS. 2A-2B, 3F-3H), which resulted in upregulation of pro-inflammatory cytokines (FIG. 5A-5D) that activated downstream anti-apoptosis and cell survival signals (FIGS. 5D, 11B-11C);

3. ER stress signaling (50-57) that inhibited canonical translation via PERK activation of eIF2α phosphorylation and promoted non-canonical translation (FIG. 2A-B), as well as activated cholesterol and lipid biosynthesis (FIGS. 6A, 6C-E, 8D, 2E)—which can increase inflammation and block apoptosis to enable chemoresistance (50-57).

Consistently, targeting the above pathways significantly curtailed chemoresistance (FIGS. 4G-4I, 10D, 5G-5H, 5K-5M, 11D, 11F-11H, 6B, 6F-6G, 12C-12D). Blocking the p38 MAPKα/β pathway with the inhibitor, LY2228820 (66, 228, 229), in combination with the anti-inflammatory Pirfenidone (49, 254-256, 260), which can target TNFα and other inflammatory factor expression (254-256) as well the inflammation regulator (44, 46-48) p38 MAPKγ isoform (45, 49, 256), prior to (and continued with) AraC chemotherapy, lead to effective loss of chemoresistance in multiple AML cell lines (FIG. 6H), and in tumors in vivo in an AML xenograft mouse model (FIG. 6L), validating their ability to reduce resistance and tumors in vitro and in vivo. Addition of lovastatin that inhibited cholesterol and its pro-inflammatory, anti-apoptotic effect (50-57), also led to similar increased chemosensitivity when combined with either or both of these anti-inflammatory and stress signaling inhibitors (FIG. 6H). The combination of Pirfenidone and LY2228820 inhibited p38 MAPKα/β as well as γ isoforms that are implicated in inflammation (44-49), where the γ isoform can be increased upon p38MAPKα/β inhibition (275), as well as inhibited downstream inflammatory effectors like TNFα (254, 255) (FIG. 5A). Therefore, the combination of Pirfenidone and LY2228820 suppressed the inflammatory and stress response more effectively, which leads to the enhanced decrease in chemoresistant cell survival and cancer persistence in vitro in cancer cell lines and in vivo mouse AML model (FIGS. 6H-K, 6L). These data indicated that blocking pro-inflammatory effectors—that are induced by DNA damage and stress signaling—led to increased chemosensitivity and decreased resistant cell survival.

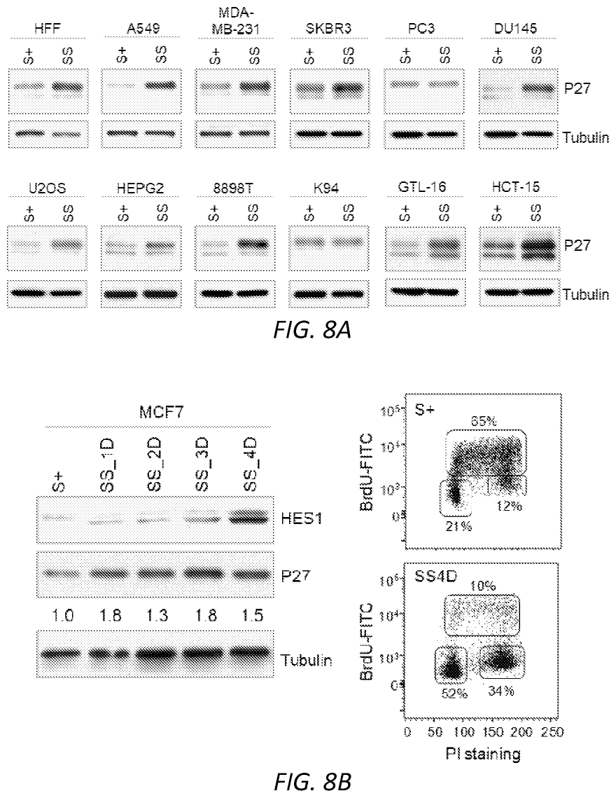

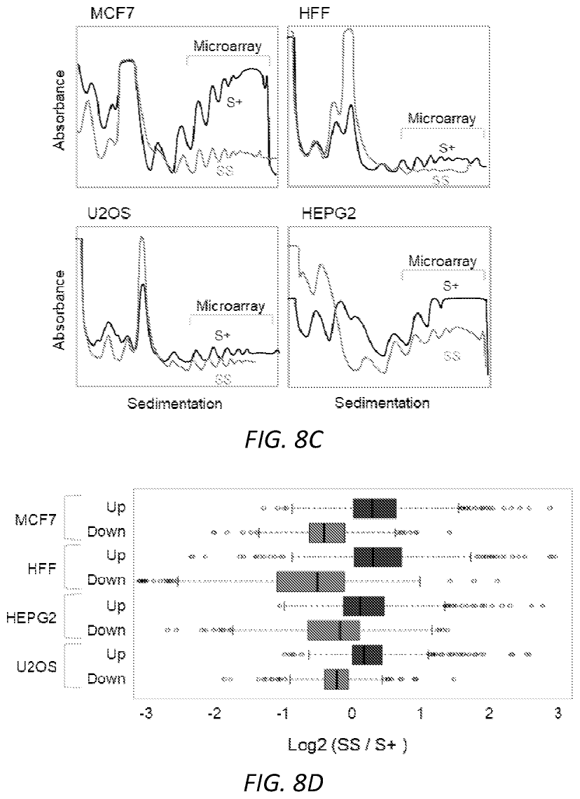

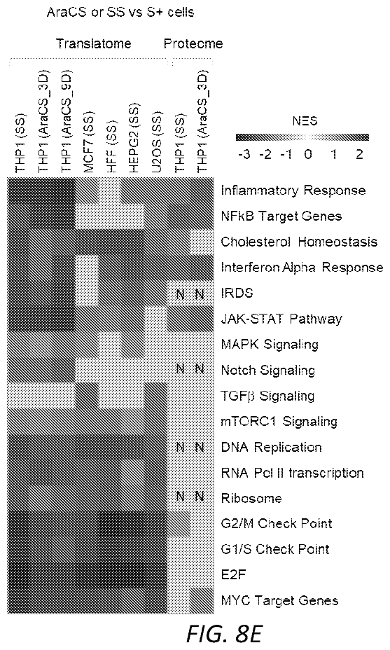

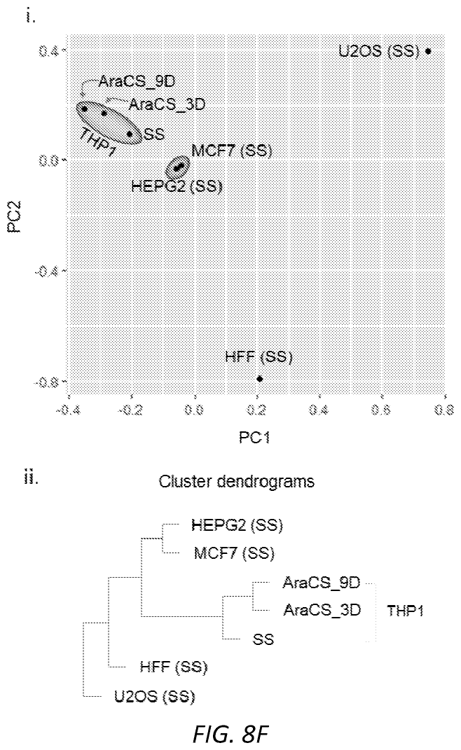

ATM and stress responsive p38 MAPK activated the STAT1/interferon response induced PKR, and along with ER stress-activated PERK, decreased canonical tRNA recruitment and translation by eIF2α phosphorylation (FIGS. 2B, 6C) (53, 114, 116, 119, 120, 125, 128, 142, 268, 269, 276, 277). ATM signaling activated AMPK, which inhibits mTOR signaling (40-42) leading to dephosphorylated and active 4EBP (FIG. 2B). ATM induced-p38 MAPK-activated STAT1 (FIGS. 4A-G, 10A-C) (45, 64, 230, 231) also increased 4EBP transcription (FIG. 2B), enhancing shutdown of canonical translation via eIF4E inhibition (127-130), which permitted non-canonical translation mechanisms (227). PERK and PKR mediated eIF2α phosphorylation as well as mTOR inhibition reduced canonical translation at the two rate limiting steps of initiation (FIGS. 2A-B, 4A-C, 10A, 6E, 12E, 13). Canonical eIF4E dependent translation promoted proliferation associated genes (109, 110, 128, 131, 158, 278). Inhibition of canonical translation via eIF2α phosphorylation by PERK and PKR led to specific mRNA translation due to altered translation initiation (109, 123, 125, 129, 132, 136, 142, 263-265), such as that of the cell cycle arrest factor, p27 KIP (279-282) (FIGS. 1C, 8A-B, p27), and GADD34 (FIG. 6D), that are reported to be translationally regulated via specialized mechanisms and alternate translation factors (109, 123, 136, 142, 263-265) that are active in G0 (31, 32). mTOR inhibition also enabled non-canonical translation (FIG. 3F-H) of other specific genes that promote resistance (119, 263, 283), including immune modulators like TNFα (FIGS. 5A-M, 11F, 3F-H), as published (32), which activates NFκB to increase survival genes (FIGS. 5D, 11B-C).

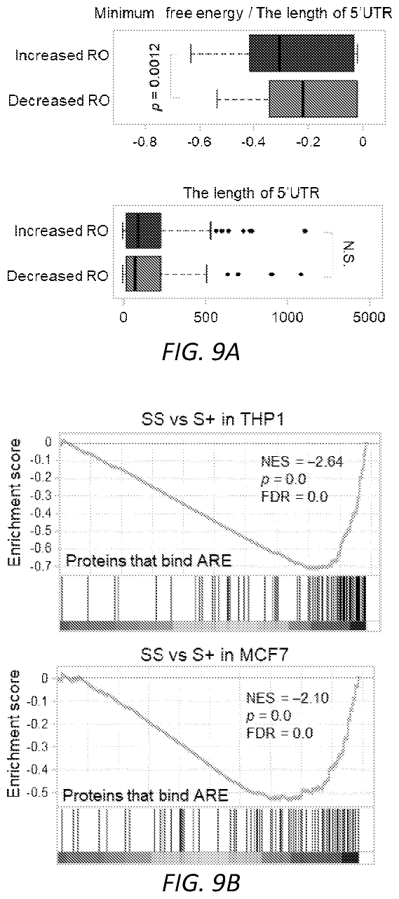

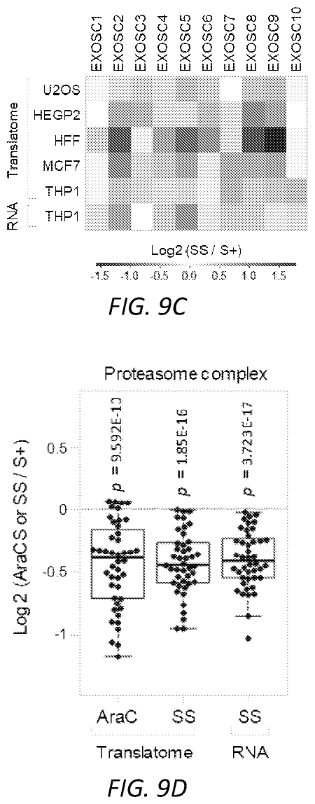

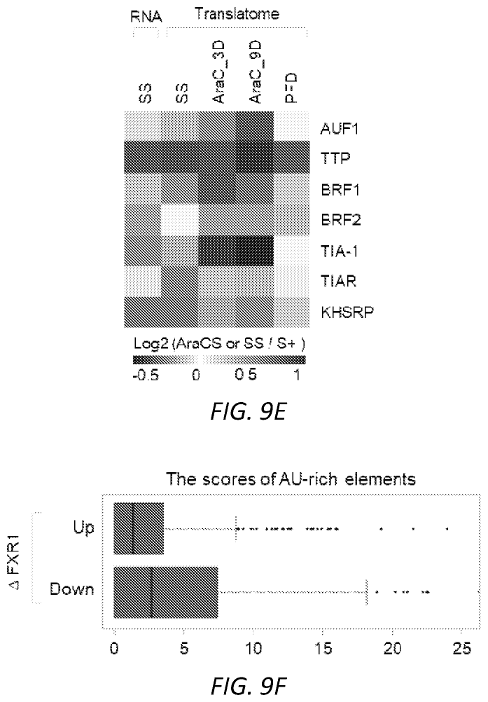

The immune genes upregulated in G0 have AREs and other UTR sequences that regulate mRNA levels and translation (FIG. 3B-C). Other sequence elements (FIGS. 3A, 9A), other factors (65), and other mechanisms (284-289) may also be involved. The ATM-p38 MAPK axis activates MK2 to stabilize these ARE bearing pro-inflammatory cytokine mRNAs by phosphorylating the mRNA decay factor, TTP to prevent its decay activity on pro-inflammatory cytokine mRNAs like TNFα (FIGS. 3D-3E, 3H, 9B-9E, 4A-4C, 4F, 5A-5C, 5E-5F). This is consistent with previous studies on the role of AREs in cancers (63, 69, 78, 290-293), and of TTP as a tumor suppressor (78, 290-293) and anti-inflammatory factor (67-73) (75, 76). Most AML cell lines tested responded to the combination treatment (LY and Pirfenidone treatment prior to and continued with AraC treatment) that inhibits the p38 MAPK/MK2 axis leading to reduced TTP phosphorylation and thus decreased TNFa (FIG. 6K); however, K562 CML cells did not respond to the combination treatment (FIG. 5K). While such non-responsive cells did show decreased MK2 phosphorylation with the combination treatment, they did not show a significant decrease in phospho-TTP levels (FIG. 6N), indicating lack of TTP decay activity (phosphorylation of TTP inhibits its activity and promotes pro-inflammatory gene expression (67-73) (184)). This may be due to other kinases that remain active in such cells and phosphorylate TTP to prevent decay of inflammatory genes by TTP, leading to resistance to the combination therapy that targets p38 MAPK/MK2 mediated inflammation. Importantly, these data suggest that the levels of phospho-TTP and thus TTP activity (phospho-TTP inactive form), may be a key indicator and regulator of pro-inflammatory gene mediated chemoresistance. In support, overexpression of a non-inhibitable form of TTP (TTP-AA), which cannot be phosphorylated and is a dominant active form that restores ARE bearing mRNA decay activity (60, 73, 75, 76, 184, 208), caused a decrease in TNFα expression levels and led to reduced chemoresistance (FIG. 6N). Together, these data suggest that phospho-TTP level is an important indicator and regulator of the inflammatory response mediated chemoresistance, which could be harnessed as a marker and target against clinical resistance.

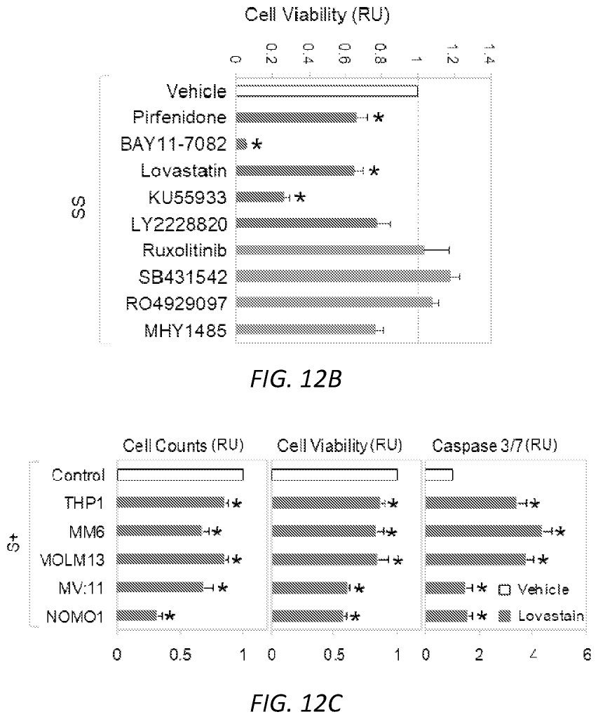

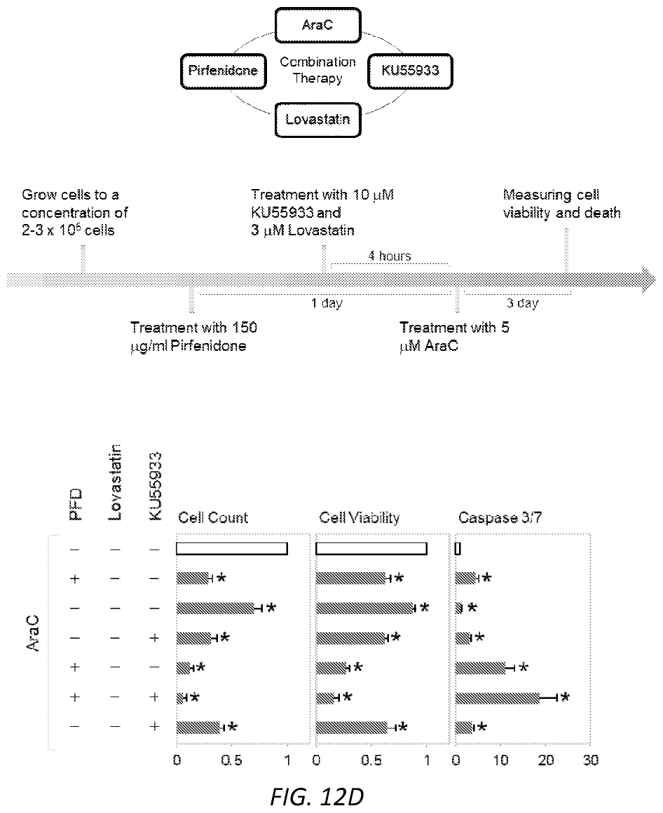

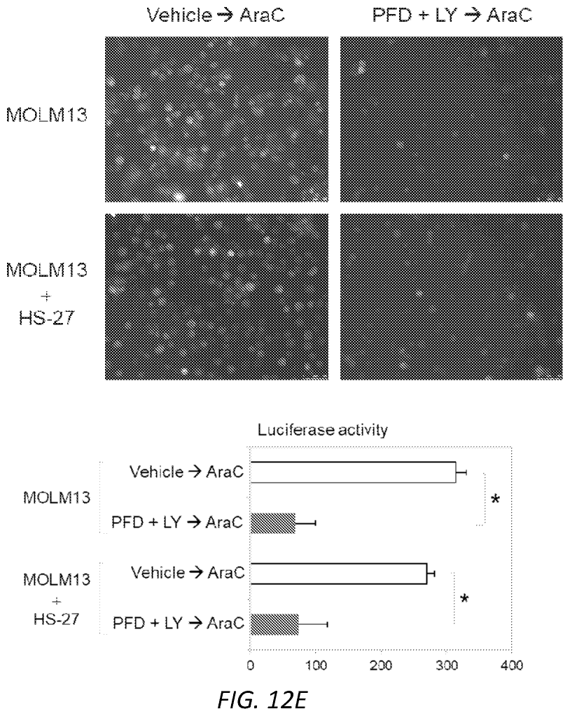

Other immune modulators include antigen presentation and processing genes like HLA-E and HLA-G (87-90) and CD47 (91-96) that are associated with resistance and immune cell modulation (97) (FIGS. 2E-2G 8D-8E, 2J-2K), as well as cell-migration inducing chemokines (FIG. 2L-2M). As shown herein, G0 resistant cells caused increased induction of cell migration by nearby cells (FIG. 2M). In addition, low mTOR activity enabled recruitment of these immune gene mRNAs by FXR1 (FIGS. 3F-3H, 9F). FXR1 mediated non-canonical translation in SS G0 cells by a mechanism (32) that was enabled by low mTOR activity/4EBP dephosphorylation, which is observed in these SS and AraCS cells (FIGS. 2B, 4A). Consistent with the current findings, depletion of FXR1 in SS G0 cells caused decreased levels of ARE bearing, cytokine and other immune gene mRNAs (FIG. 9F). In accord with the role of such immune genes in induction of monocyte migration (FIGS. 2L-M), depletion of FXR1 led to reduced induction of nearby monocyte cell migration (157). These data, along with increased cell adherence (148-151) (FIGS. 2E, 2H-I), suggest that G0 resistant cells upregulate genes that can communicate with their environment, which could promote survival (43, 146, 147). Together, these pathways upregulated in resistant cells (FIGS. 4A, 5A, 6C) decrease canonical translation and permit non-canonical translation and post-transcriptional regulation of specific genes (FIG. 12E, model in FIG. 13) to induce a pro-inflammatory response that promotes chemotherapy survival of G0 cancer cells.

Methods of Treatment

The methods described herein can be used for the treatment of disorders associated with abnormal apoptotic or differentiative processes, e.g., cellular proliferative disorders or cellular differentiative disorders, e.g., cancer, e.g., by producing an active or passive immunity. Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In preferred embodiments, the proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the disorder is acute myelogenous leukemia (AML). Additional exemplary myeloid disorders include, but are not limited to, acute promyeloid leukemia (APML), and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the subject has a disorder that is associated with increased levels of phosphorylated tristetraprolin (TTP; also known as ZFP36, NUP475 and GOS24) and/or reduced levels of TTP activity (as phospho-TTP is the inactive form). Thus, in some embodiments, the methods include obtaining a sample comprising cancer cells from the subject and detecting a level of phospho-TTP and/or levels of TTP activity using a method known in the art (e.g., as described in Kesarwani et al., Nat Med. 2017 April; 23(4):472-482; Taylor et al., The Journal of Biological Chemistry 270:13341-13347 (1995); Clement et al., Mol Cell Biol. 2011 January; 31(2): 256-266; and Kedar et al., PLoS One. 2010; 5(3): e9588); Tiedje et al., Nucleic Acids Research, September 2016, 44(15):7418-7440. The level of phospho-TTP or TTP activity is compared to a reference level, e.g., a reference level that represents the level of phospho-TTP or TTP activity in non-chemresistant cancer cells, and if the level of phospho-TTP in the sample is above the reference level, or the level of TTP activity in the sample is below the reference level, then the subject is selected for treatment using a method described herein, and optionally administered that treatment. In some embodiments, if the level of phospho-TTP in the sample is above the reference level, or the level of TTP activity in the sample is below the reference level, then the subject is selected for inclusion and/or treatment in a clinical trial of a method described herein. An exemplary reference sequence for human TTP (also known as mRNA decay activator protein ZFP36) is in GenBank as NP_003398.2, and antibodies that bind to TTP are commercially available, e.g., from EMD Millipore, Invitrogen, Sigma-Aldrich, and AbCam, among many others.

As one of skill in the art will appreciate, the reference levels can vary depending on the analysis method used. Thus, in the present methods a reference value determined in a subject should be compared to a reference value determined using the same analysis method. One of skill in the art would readily be able to determine such a reference value. Suitable reference values can include a single cut-off (threshold) value, such as a median or mean, or a level that defines the boundaries of an upper or lower quartile, tertile, or other segment of a clinical trial population that is determined to be statistically different with respect to levels of phospho-TTP or TTP activity from the other segments. It can be a range of cut-off (or threshold) values, such as a confidence interval. It can be established based upon comparative groups, such as where a level in one defined group is a fold higher, or lower, (e.g., approximately 2-fold, 4-fold, 8-fold, 16-fold or more) than the level in another defined group. It can be a range, for example, where a population of subjects (e.g., control subjects) is divided equally (or unequally) into groups, such as a low-level group, a medium-level group and a high-level group, or into quartiles, the lowest quartile being subjects with the lowest levels and the highest quartile being subjects with the highest levels, or into n-quantiles (i.e., n regularly spaced intervals) the lowest of the n-quantiles being subjects with the lowest levels and the highest of the n-quantiles being subjects with the highest levels.

The methods described herein include administration of a combination of treatments. First, the method includes administering a treatment that reduces chemoresistance, e.g., an inhibitor of p38 MAPK (also known as p38a), e.g., selected from the group consisting of SB203580; Doramapimod (BIRB 796); SB202190 (FHPI); Ralimetinib (LY2228820); VX-702; PH-797804; VX-745; TAK-715; Pamapimod (R-1503, Ro4402257); BMS-582949; SB239063; Losmapimod (GW856553X); Skepinone-L; Pexmetinib (ARRY-614). The p38 MAPK family has four members, including MAPK11, MAPK12, MAPK13, and MAPK14, which encode the p3803 MAPK, p38γ MAPK, p386 MAPK, and p38a MAPK isoforms, respectively. In some embodiments, the methods include administering an inhibitor or combination of inhibitors that target p38a and p38γ. In some embodiments, the inhibitor of p38 MAPK is an inhibitor of MAPK14 (e.g., 4-Hydroxyquinazoline (Enzo Life Sciences, Inc.); AL 8697 (Tocris Bioscience); AMG 548 (Amgen, Tocris Bioscience); AMG-47a (Biorbyt); ARRY-797 (ARRY-371797; Array Biopharma); Doramapimod (BIRB-796; Beohringer Ingelheim Pharmaceuticals); CGH 2466 dihydrochloride (Tocris Bioscience); CMPD-1 (Tocris Bioscience); CV-65 (Abcam); D4476 (BioVision, Biorbyt); DBM 1285 dihydrochloride (Tocris Bioscience); Doramapimod (BioVision); EO 1428 (Tocris Bioscience); JX-401 (Enzo Life Sciences, Inc.); Losmapimod/GW856533X (GlaxoSmithKline); ML 3403 (Tocris Bioscience); p38/SAPK2 Inhibitor (SB 202190) (MilliporeSigma); Pamapimod (R-1503, R04402257; Roche); PD 169,316 (Enzo Life Sciences, Inc.); R1487 (Biorbyt); Saquayamycin B1 (Abcam); SB 202190 (BioVision, Tocris Bioscience); SB 203580 (BioVision, InvivoGen); SB 706504 (Tocris Bioscience); SB202190. Hydrochloride (Enzo Life Sciences, Inc.); SB203580 (Enzo Life Sciences, Inc.); SB220025 (Enzo Life Sciences, Inc.); SB239063 (Enzo Life Sciences, Inc.); SB242235 (Biorbyt); SCIO-323, SCIO 469 (Scios Inc, Tocris Bioscience); SD-169 (Enzo Life Sciences, Inc.); SKF 86002 dihydrochloride (Tocris Bioscience); SX 011 (Tocris Bioscience); TA 01 (Tocris Bioscience); TA 02 (Tocris Bioscience); TAK 715 (Biorbyt, Tocris Bioscience); VX-745 (Neflamapimod; Biorbyt, Tocris Bioscience); or VX-702 (Abcam, BioVision)).

In some embodiments, e.g., where an inhibitor of p38 MAPK is used that does not inhibit p38γ (e.g., ralimetinib), the methods include administering an anti-inflammatory agent such as Pirfenidone (49, 254-256, 260; Cho and Kopp, Expert Opin Investig Drugs. 2010 February; 19(2): 275-283), which can target both soluble and trans-membrane TNFα and other inflammatory factor expression (254-256) as well the inflammation regulator (44, 46-48) p38 MAPKγ isoform (45, 49, 256).

After administration of the p38 inhibitor, the methods include administration of a chemotherapy. Suitable chemotherapies that can be used in the present methods, e.g., for treating leukemias such as AML or solid tumors including breast cancer, can include Cytarabine (arabinosylcytosine cytosine arabinoside, ara-C, or CYTOSAR) and anthracycline drugs such as Daunorubicin (daunomycin or CERUBIDINE) or doxorubicin (ADRIAMYCIN), idarubicin, and mitoxantrone; others can include Cladribine (LEUSTATIN, 2-CdA); Fludarabine (FLUDARA); Topotecan; Etoposide (VP-16); 6-thioguanine (6-TG); Hydroxyurea (HYDREA); Methotrexate (MTX); 6-mercaptopurine (6-MP); or Azacitidine (VIDAZA). Others (e.g., for treating ALL) can include Decitabine (DACOGEN), Vincristine (ONCOVIN) or liposomal vincristine (MARQIBO); L-asparaginase (ELSPAR) or PEG-L-asparaginase (pegaspargase or ONCASPAR); Teniposide (VUMON); Cyclophosphamide (CYTOXAN); Prednisone; or Dexamethasone (DECADRON). Other chemotherapy agents are known in the art. In some embodiments, the chemotherapy agent is not a smac-mimetic (66).

In some embodiments, a cholesterol inhibitor is also administered with the p38 inhibitor, e.g., a statin drug such as atorvastatin, cerivastatin, fluvastatin, lovastatin, pitavastatin, pravastatin, rosuvastatin, simvastatin, and analogs thereof.

In some embodiments of the present methods, the chemotherapy is administered after, e.g., between 2-24, 4-24, 4-12, 4-8, −4-6 hours after, administration of at least one dose of the p38 inhibitor. In some embodiments of the present methods, at least one dose of the p38 inhibitor is administered before, e.g., between 2-24, 4-24, 4-12, 4-8, −4-6 hours before, administration of the first dose of chemotherapy, and optionally is also administered concurrently therewith, (e.g., at the same time as, or 1-24 hours (e.g., 4-24, 4-12, 4-8, or 4-6 hours) before, one or more additional doses of chemotherapy).

Pharmaceutical Compositions and Methods of Administration

Also provided herein are pharmaceutical compositions comprising agents described herein as active ingredients, and methods of use thereof. In some embodiments, the compositions include Ralimetinib (LY2228820) and pirfenidone as active agents.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

Materials and Methods

The following materials and methods were used in the Examples, below.

Cell Culture

THP1 cells were cultured in Dulbecco's modified Eagle medium (RPMI)1460 media supplemented with 10% fetal bovine serum (FBS), 2 mM L-Glutamine, 100 g/mL streptomycin and 100 U/ml penicillin at 37° C. in 5% CO₂. SS THP1 cells were prepared by washing with PBS followed by serum-starvation at a density of 2×10⁵ cells/mL and AraCS cells, by treatment with 5 μM AraC for 3 days or 9 days. MCF7, HFF, HEPG2 and U2OS cells were cultured in Dulbecco's modified Eagle medium (DMEM) media with 10% FBS, 2 mM L-Glutamine, 100 μg/mL streptomycin and 100 U/ml penicillin, as done previously (1, 2). THP1 (TIB-202), MV4:11 (CRL-9591), K562 (CCL243), HFF (SCRC-1041), MCF (HTB-22), U2OS (HTB-96) and HEPG2 (HB-8065) were obtained from ATCC. MOLM13 (ACC554), NOMO1 (ACC542) and MONOMAC6 (ACC124) were obtained from DSMZ. Cell lines kindly provided from the Scadden group (3) and MOLM13-GFP-Luc from Monica Guzman (4) were tested for Mycoplasma (Promega) and authenticated by the ATCC Cell Authentication Testing Service (3).

Primary AML Patient Samples and Human Monocytes

Primary AML cells were collected using a protocol approved by the Partners Human Research Committee Institutional Review Board. AML samples used in this study were MGH15—bone marrow 60% blasts, karyotype 46, XX,t (9; 11) (p22; q23)[20/20]; MGH22—peripheral blood, 60% blasts, karyotype 46,XX,t (3; 21) (q26; q22),t (9; 22) (q34; q11.2)[18]/46,XX[2]; and MGH25—bone marrow, 90% blasts, karyotype 46,XX[20]. Bone marrow or peripheral blood mononuclear cells were isolated from de novo AML patients by ficoll density gradient centrifugation and cryopreserved with DMSO in a liquid nitrogen tank. Thawed cells were maintained in RPMI media with 10% FBS for several days before drugs treatment and analyses. Human CD14+ (2W-400C) and CD34+ monocytes (2M-101) were obtained from Lonza.

In Vivo Xenograft AML Mouse Model

MOLM13 xenograft AML mouse model was created by injecting MOLM-13 cells into the flanks of nod-scid mice (obtained from MGH Cox-7 Gnotobiotic animal facility of the AAALAC-accredited Center for Comparative Medicine and Services at MGH). Mice were treated with pirfenidone (150 mg/kg, intraperitoneally) plus LY2228820 (20 mg/kg, intraperitoneally) or vehicle 1-hour prior to AraC (160 mg/kg, intraperitoneally) injection every two days for 8 days. Tumor volumes were measured at indicated time points.

Polysome Profiling with Microarray

Sucrose was dissolved in lysis buffer containing 100 mM KCl, 5 mM MgCl₂, 100 μg/ml cycloheximide, 2 mM DTT and 10 mM Tris-HCl (pH 7.4). Sucrose gradients from 15% to 50% were prepared in ultracentrifuge tubes (Beckman) as previously described (1, 5-7). Cells were treated with 100 μg/mL cycloheximide at 37° C. for 5 minutes before collecting them. Harvested cell were rinsed with ice-cold PBS having 100 μg/mL cycloheximide and then were resuspended in lysis buffer with 1% Triton X-100 and 40 U/mL murine (New England Biolabs) for 20 minutes. After centrifugation of cell lysates at 12,000×g for 20 minutes, supernatents were loaded onto sucrose gradients followed by ultracentrifugation (Beckman Coulter Optima L90) at 34,000×rpm at 4° C. for 2 hours in the SW40 rotor. Samples were separated by density gradient fractionation system (Teledyne Isco). RNAs were purified by using TRIzol (Invitrogen) from heavy polysome fractions and whole cell lysates. The synthesized cDNA probes from WT Expression Kit (Ambion) were hybridized to Gene Chip Human Transcriptome Array 2.0 (Affymetrix) and analyzed by the Partners Healthcare Center for Personalized Genetic Medicine Microarray facility. Gene ontology analysis for differentially expressed translatome or proteome was conducted by DAVID 6.7 tools (8) (9). Molecular signatures enriched in AraCS or SS were identified by GSEA (10).

Plasmids

TRIPZ plasmids expressing shRNA against human TNFα (V2THS_111606), miR30a primiR sequences used as control (RHS4750), and human S100A12 (TRCN0000053904) were obtained from Open Biosystems and MGH cancer center, respectively. Stable cell lines were constructed as described by Open Biosystems. The stable cells expressing shRNA against TNFα were induced with 1 μg/mL doxycycline at indicated time points to knockdown TNFα. Cells were treated with 10 ng/ml recombinant TNFα (R&D Systems) to activate NFκB pathway. Myc-tagged TTP-AA (11, 12) was a gift from Nancy Kedersha and Shawn Lyons from Paul Anderson's lab.

MTS Assay

MTS assay, a colorimetric quantification of viable cells was conducted as described by the manufacturer, Promega. A volume of 100 μl cells was placed in a 96-well plate after drugs treatment. A volume of 20 μl MTS reagent (CellTiter 96® Aqueous Non-Radioactive Cell Proliferation Assay) was added to each well followed by incubation at 37° C. for 1 hour. Absorbance was measured at 490 nm by using a microplate reader.

Caspase 3/7 Assay

After drugs treatment, cell death was measured by using caspase-glo® 3/7 assay kit (Promega) according to the protocol provided by the manufacturer. The equal volume of caspase-glo reagent was added to cells, and samples were gently mixed with pipetting. The plates were incubated at room temperature in the dark for 2 hours. The luminescence of each sample was measured in a luminometer (Turner BioSystems).

Flow Cytometry and Cell Cycle Analysis

Cell proliferation was determined by flow cytometry of cells labeled with propidium iodide and bromodeoxyuridine (BrdU). The cells were incubated with 10 μM BrdU for 90 minutes at 37° C. in 5% CO₂ before harvesting. Collected cells were fixed in ice cold 70% ethanol overnight. Cells were washed in PBS and treated with 2 M HCl for 30 min. Cells were incubated for 1 hour with anti-BrdU antibody conjugated to FITC (eBioscience) in the dark, washed and stained with propidium iodide. Samples were filtered through a nylon mesh filter and cell cycle analysis performed on the flow cytometry (13).

Western Blot Analysis

Cells were collected and resuspended in lysis buffer containing 40 mM Tris-HCl (pH 7.4), 6 mM MgCl₂, 150 mM NaCl, 0.1% NP-40, 1 mM DTT and protease inhibitors (Roche). Separation of whole cell lysate into cytoplasmic and nuclear fractions, and immunoprecipitation of FXR1 were conducted as previously described (2, 14). Samples containing 80 μg of protein were loaded onto 10% or 12% SDS-PAGE (Bio-Rad), transferred to PVDF membranes and processed for immunoblotting. Antibodies against p27 (#06-445), tubulin (#05-829) and FXR1 were obtained from Millipore. Antibodies against HES1 (# sc-25392), eIF2α (# sc-11386) and phospho-4EBP1 were from Santacruz. Antibodies against phospho-ATM (# ab81292), phospho-PKR (# ab32036) and phospho-IRE1 (# ab124945) were from Abcam. Antibodies against XBP1 (#619501) and phospho-PERK (#649401) were from Biolegend. Antibodies again TNFα (#3707), phospho-p38 MAPK (#4511), phospho-MK2 (#3007), phospho-STAT1 (#9167), STAT1 (#9172), phospho-eIF2α (#9721), TTP (##71632) and 4EBP1 were from Cell Signaling Technology. FXR1, 4EBP1, and p-4EBP1 antibodies were used previously (1, 2).

Cell Adhesion Assay

A 24-well plate was coated with 5 μg/ml human fibronectin (BD Biosciences) for 2 hours at 37° C. S+ and SS cells were washed PBS and resuspended in media with 10% FBS. Cells were added into a 24-well plate at a density of 1×10⁵ cells/well and incubated for 2 hours at 37° C. in 5% CO₂. The plate was washed with PBS to remove nonadherent cells, and adherent cells were stained with 0.2% crystal violet in 10% ethanol for 10 minutes. Microscopy images were taken, and the number of adherent cells on the plate were determined (15).

Apoptosis Analysis

Leukemic cells were treated with indicated drug combinations. Annexin V FITC/PI staining was performed with FITC Annexin V Apoptosis Detection Kit I (BD Pharmingen). Flow cytometry analysis and FlowJo software were used to quantitate the percentages of apoptotic cells.

Colony Forming Assay

After treatment with indicated chemicals, the same number of cells were plated in methylcellulose-based media with human recombinant cytokines (stem cell technology, MethoCult™ H4435). Number of colonies was quantitated in each plate after 10 days.

Transwell Cell Migration Assay

Transwell chambers (8 μm pore, Corning) were pre-equilibrated with serum-free media for 1 hour. GFP-tagged THP1 or MCF7 cells (2×10⁴/chamber) were placed in the top chamber, and 700 μL of S+ or SS THP1 cells containing media, in the bottom chamber. The bottom chamber with SS THP1 cells was supplemented with 10% FBS as a control. The chambers were incubated at 37° C. for 4 hours in 5% CO₂. Cells on the upper surface of the filter were removed with a cotton swab. Migrated MCF7 cells on the underside of the filter were fixed in formaldehyde for 10 minutes and subsequently stained with 0.2% crystal violet. Migrated GFP-tagged THP1 cells were observed in the bottom chamber and visualized using a microscope. Microscope images were taken and the numbers of migrated cells were determined (15-17).

Mass Spectrometry

Multiplex quantitative proteomics analysis was conducted, as previously (18), from S+, SS and AraC treated THP1 leukemic cells.

Inhibitors

Pirfenidone (10 to 300 μg/ml (19)) was obtained from Chemietek. AraC (1 to 10 μM (20)), Lovastatin (0.1 to 100 μM (21)), LY2228820 (0.03 to 2 μM (22)), BIRB796 (BIRB, 5 μM (23-27)), and SB431542 (2 to 10 μM (28)) were from Selleckchem. KU55933 (10 μM (29)), BAY 11-7082 (10 μM (30)), Ruxolitinib (0.156 to 5 μM (31)), R04929097 (10 μM (32)), and MHY1485 (2 μM (33)) were from Cayman Chemical and Doxorubicin (10 to 500 nM (34)) was from Tocris Bioscience.

Motif Analysis

The Multiple Em for Motif Elicitation (MEME) software was used to search for cis-elements enriched in 5′ UTR of translationally regulated genes (35). Human 5′ UTR sequences were retrieved from UCSC table browser (36). In a discriminative mode, 5′ UTR sequences of translationally up- or down-regulated genes were used as the primary sequences and 5′ UTR sequences of translationally unchanged genes, the control sequences. Motifs were found in the given strand with 6-30 nt motif width.

Statistical Analyses

All experiments in every figure used at least 3 biological replicates except for microarray, mass spectrometry, and patient sample data. Each experiment was repeated at least 3 times. No statistical method was used to pre-determine sample size. Sample sizes were estimated on the basis of availability and previous experiments (1, 2). No samples were excluded from analyses. P values and statistical tests were conducted for each figure. Statistical analyses were conducted using R or Excel. Two-tailed unpaired t-test or Wilcoxon rank sum test was applied to assess statistical significance. SEM (standard error of mean) values are shown as error bars in all figures. Means were used as center values in box plots. P-values less than 0.05 were indicated with an asterisk. E-values were used for the statistical significance in the motif analysis.

### Example 1. G0 THP1 Leukemic Cells, Induced by Serum-Starvation, are Chemoresistant, and Express a Specific Proteome-Similar to Cells Isolated after Surviving Chemotherapy

To study clinical resistance in cancer, THP1 human acute monocytic leukemic cells were selected as they show significant chemoresistance to cytarabine (103-105) (cytosine arabinoside, AraC, FIG. 7A), a standard anti-leukemic chemotherapy (106, 107) that targets proliferating cells (referred to as S+). Serum-starvation of THP1 cells (31) and certain cell lines (3, 16, 27, 33) can induce a transient G0 arrest state and show known G0 and cell cycle arrest markers (FIG. 1B-D). Such serum-starvation induced G0 cells (referred to as SS) could be returned to the cell cycle upon serum addition (FIG. 1E), verifying that they were quiescent and transiently arrested compared to alternative arrested states—of senescence or differentiation—that are not easily reversed (3). Interestingly, consistent with the temporary loss of proliferation, we found that serum-starvation induced G0 SS cells showed resistance to AraC chemotherapy—that targets proliferating cancer cells. Serum-grown proliferating S+ cells showed a dose dependent decrease in cell viability as expected while serum-starved SS cells persist, indicating their chemoresistance (FIG. 1F).

Chemoresistant cancer cells include cancer stem cells, and are a subpopulation that can be isolated from cancers after treatment with chemotherapy (4-7, 12, 13, 15-26). Proliferating cancer S+ cells are eliminated by the proliferation-targeting chemotherapeutic-leaving behind the subpopulation of chemoresistant cancer cells that can be isolated because they are not targeted by the chemotherapeutic as they are temporarily not proliferating. We found that AraC surviving THP1 (chemoresistant/resistant, referred to as AraCS) cells were transiently arrested, like serum-starved SS G0 cells (FIG. 7B). AraC surviving AraCS cells recovered from their transient arrest upon AraC removal and proliferate (FIG. 1G), as well as responded when re-exposed to chemotherapy again (FIG. 1H), affirming the reversible arrest state of resistant cells, similar to serum-starvation induced G0 SS cells (11-14) (1, 2, 4-7, 15-26).

Translation is altered in SS leukemic cells with inhibition of the two, key rate-limiting steps of canonical translation initiation, which permits non-canonical mechanisms and specific gene translation to occur (31, 32). We found that AraCS cells showed similar inhibition of canonical translation (FIG. 1I), suggesting that a distinct translated gene expression program may commonly underlie both chemoresistant populations. Therefore, we profiled chemosurviving AraCS cells (that show transient proliferation arrest, FIG. 7B), as well as serum-starvation induced G0 arrested SS cells that are chemoresistant (FIG. 1F), and compared them with proliferating S+ cells at the RNA, proteome and translatome levels, using microarray, multiplexed quantitative proteomics (32, 108), and polysome profiling by microarray analysis (FIGS. 1A, J, 7C). Consistently, we found that serum-starvation induced G0 SS cells that exhibited chemoresistance showed significantly similar proteomes—in the specific genes altered over proliferating S+ cells—to that of chemosurviving AraCS cells isolated after AraC chemotherapy (FIG. 1K). These data suggested that when these cancer cells become chemoresistant, such cells exhibit a common set of specific genes expressed at the protein level, which is distinct from proliferating cells.

### Example 2. Translation Profiling Analysis of SS and AraCS Resistant Cells, Compared to S+ Cells

In parallel to analysis of the proteome, we analyzed the translatome by polysome profiling and microarray analysis of heavy polysome-associated mRNAs (31, 109-113) in: SS THP1 cells that were induced to G0 by serum-starvation from 4 h to 4 days, in chemosurviving AraCS THP1 cells that were isolated from proliferating S+ THP1 cells after AraC treatment from 3 and 9 days, and in untreated proliferating S+ THP1 cells (FIGS. 1A,7C). We also examined the transcriptome by microarray to differentiate translationally-regulated from transcriptionally-regulated genes.

Comparison of the transcriptome and translatome of SS cells revealed a significant proportion of genes that were translationally regulated and did not overlap with the transcriptome (FIG. 1L). SS cells show similar translatome to that of AraCS cells—in the specific genes altered over S+ cells—with extended AraC treatment (3 days versus 9 days) having a similar translation profile (FIG. 1M-N). These data indicated the relevance of examining the translatome along with the transcriptome, and suggested that cancer cells express specific genes when they become chemoresistant, which are distinct from gene expression in proliferating cells.

A comparison of different durations of serum-starvation revealed that early serum-starved G0 (4 h and 1 day SS) cells were distinct from late, 2-4 day SS G0 cells (2-4 day SS were more similar in their translatomes, R²=0.81), indicating temporally regulated levels of gene expression in G0 (FIG. 7D-Ei-iii). This was consistent with previous findings that concluded that G0 is a continuum of assorted arrested states (3), with temporal differences in underlying gene expression at early times, in early G0 compared to lesser differences in late G0. Since chemosurviving cells are known to include cancer stem cells (4-7), we compared our datasets with published transcription profiles from other studies on cancer stem cells in leukemia including: leukemia stem cells (LSC) (36) from AML, dormant leukemic cells (LRC) and minimal residual disease (MRD) from chemotherapy surviving patient samples (35) with acute lymphocytic leukemia (ALL), as well as SS G0 fibroblast cells (G0 HFF) (3) that were isolated by different methods. Consistently, we found that genes upregulated in our SS and AraCS resistant THP1 cells include these published gene signatures on leukemic stem cells (FIGS. 1O-Q, 7F). These data indicated that chemosurviving and G0 THP1 AML cells, expressed similar genes as cancer stem cells and G0, representing a common G0 gene expression signature.

Similar global translatome analyses were conducted in other tumor cell lines that were serum-starved, to obtain SS G0 cells that express cell cycle arrest markers (FIG. 8A-B)—including breast (MCF7), liver (HEP-G2), and osteosarcoma (U2OS) as well as non-cancerous serum-starvation induced G0 fibroblasts (HFF) (FIG. 8C)—and thereby, identified gene categories and pathways that were commonly upregulated and downregulated in G0 cells from multiple cell lines (FIG. 8D).

### Example 3. Altered Translation with Inhibition of Canonical Translation Mechanisms in SS and AraCS Cells

We investigated the mechanisms of gene expression regulation that allow for specific genes to be expressed while others are repressed in SS and AraCS cells. There are two rate limiting steps in canonical translation initiation: mRNA cap recognition to bring in the ribosome to the mRNA, and recruitment of the initiator tRNA (FIG. 2A). The recruitment of the initiator tRNA by eIF2 can be blocked by eIF2α phosphorylation as a stress response by one of four specific kinases, limiting translation in G0, and leading to non-canonical translation mechanisms as we and others showed previously (31, 114-126). We found that PKR and PERK, two of the eIF2 kinases, were activated and led to increased phosphorylation of eIF2c (FIGS. 2A, 2B upper panel) to inhibit canonical translation initiation. Our previous data had also revealed decreased mTOR activity (32), leading to dephosphorylated and active eIF4EBP (4EBP) (FIGS. 2A, 2B lower panel) that inhibited eIF4E interaction with eIF4G, and thus reduced canonical cap dependent translation at the other rate-limiting step in translation initiation (127-131, 131-134). Decreased mTOR activity reduced Terminal oligopyrimidine tract (TOP) mRNA translation, including ribosomal protein genes (135-138, 138-141); accordingly, we found decreased translation of ribosomal protein genes, and validated TOP mRNAs (FIG. 2C). The decreased canonical translation in resistant cells would enable non-canonical translation (119, 130) mediated by alternative factors (117, 142), as observed previously in G0 (31, 32). Consistently, these conditions of decreased canonical translation were accompanied by a significant number of genes that are upregulated translationally and at the protein level (FIG. 2D), indicating that non-canonical translation mechanisms were enabled in these conditions of decreased canonical translation and led to specific gene expression.

### Example 4. Molecular Signatures Enriched in the Translatome and Proteome of Chemoresistant G0 Cells

Common pathways and gene categories that correlated with chemoresistance were identified by comparing the translatomes of SS and AraCS THP1 cells, and other solid tumor SS cells compared to that of S+ cells, using GSEA and DAVID tools (FIG. 8E), as well as PCA and unbiased hierarchical clustering analysis (FIG. 8F). The analysis revealed genes that were hypothesized to decrease with cell cycle arrest, such as those involved in DNA replication and the cell cycle (FIGS. D, 2E). Common gene categories upregulated in the translatomes of SS and AraCS THP1 cells were validated by quantitative proteomics and revealed the top common categories in the G0 translatome that correlate with that of the chemoresistant state—immune response genes that included inflammatory response genes and immune modulators/antigen presentation and processing genes, as well as other categories including cell adhesion, cell migration, and lipid biosynthesis and cholesterol pathway (FIG. 2E).

### Example 5. Translational Regulation of Specific mRNA Expression in SS and AraCS Cells

To identify the genes that are translationally regulated in G0, we compared the polysome associated mRNAs with their total RNA levels in serum-starved cells to generate the change in ribosome occupancy (ΔRO) (143-145)—which is the ratio of the relative amount of mRNA that is associated with heavy polysomes compared to the total mRNA level of each gene (FIG. 2F). These data revealed that distinct proportions of genes were translationally regulated while others were regulated in coordination with changes in the RNA level. Translationally upregulated genes included the top upregulated genes in G0 resistant cells, such as immune response/inflammatory genes and cell adhesion genes. Translationally downregulated genes included RNA processing, and ribosome genes, consistent with the limited, specialized gene expression in such cells, as well as decreased DNA repair genes that would permit DNA damage and stress signaling (FIG. 2G).

### Example 6. Upregulation of Cell Adhesion, and Immune Modulator Genes Result in Increased G0 Cell Adherence, and Induction of Cell Migration by G0 THP1 Leukemic Cancer Cells

We observed that the genes upregulated include cell adhesion factors, and immune cell modulators-including antigen presentation (and processing) genes, inflammatory response genes, and cell migration promoting factors (FIG. 2E-G)—indicating that G0 resistant cells promoted genes that interact with the external environment/neighboring cells to enable their persistence, as previously observed with tumor-stromal interactions (43, 146, 147). Consistent with the increased cell adhesion gene expression (FIGS. 2H, 2F-G, 8E) that are important for G0 arrest, LSCs (148) and chemoresistance (43, 146, 147) and tumor progression (148-151), we found that SS G0 THP1, and MCF7 cells were more adherent on fibronectin coated plates (FIG. 2I). G0 resistant cells promote production of immune modulators, antigen processing and presentation genes (FIGS. 2E-G, 8E) that can regulate the anti-tumor immune response (152-156). These include specific HLA/MHC genes that were translationally upregulated (HLA-E, HLA-G, FIG. 2J), and are associated with resistance (87-90) and receptors such as CD47 (FIG. 2K) that modulate immune cells (91-96), are expressed in leukemic stem cells, and are poor prognostic factors for AML survival (97). Cytokines and chemokines (98-102) that induce cell migration (FIG. 2L) were also increased in G0 resistant cells. We tested induction of cell migration of a monocyte cell line that stably expresses GFP (GFP-THP1), and of a breast cancer cell line (MCF7), using a transwell assay (157)—with the inducing lower cell chamber containing S+ and SS THP1 cells to test their ability to induce migration. Compared to S+ cells, SS G0 THP1 cells (with serum added during the test to both compartments to equalize serum content) showed increased induction of cell migration of GFP-THP1 and MCF7 cells (FIG. 2M) (157). These data indicated that G0 resistant cells were more adherent than proliferating cells and caused induction of cell migration of nearby cells, which can impact their persistence (43, 98-102, 146, 147).

### Example 7. Post-Transcriptional and Translational Regulation of Specific mRNAs in SS and AraCS Cells

We analyzed the untranslated regions (UTRs) of these genes to identify motifs that lead to specific mRNA gene expression at the post-transcriptional and translation levels. The translationally upregulated genes enrich for a 5′-UTR GC rich motif, while genes that are translationally repressed enrich for an AT rich motif (FIGS. 3A, 9A). These are distinct from other 5′-UTR elements that are associated with specific cap binding proteins (110, 158) but are similar to related motifs found in tumor initiating cells (118), where canonical translation is reduced by eIF2α phosphorylation and non-canonical translation by an alternative factor occurs. Consistently, our gene sets revealed that translationally upregulated mRNAs tended to have enrichment of GC-rich, highly structured 5′-UTRs with large free energies of folding (FIG. 9A), and our previous data had similarly revealed an alternative, non-canonical translation factor in G0 cells (31), compensating for the eIF2α phosphorylation (31, 114-126).

A second category of specific regulatory elements that we found on genes with increased mRNA and translation levels were 3′-UTR AU-rich elements (AREs, FIG. 3B-C). AREs are mRNA stability and translation regulatory elements that control expression of critical growth factors, oncogenes, and immune genes (58, 67, 159-165). Many of the immune modulators, including antigen presentation genes, inflammatory response genes, and cell migration promoting chemokines (FIGS. 2E-G, 2J-M, 3B-C) have AREs and are regulated by such UTR elements (166-168). AREs are generally involved in mediating specific mRNA decay; however, depending on their associated RNA binding proteins and cellular conditions that modify these interactions, AREs can mediate stabilization and translation of mRNAs, as in the case of ARE bearing TNFα pro-inflammatory cytokine and other mRNAs, in G0 and in other conditions (112, 160, 169-184). Consistently, we found that many factors that are implicated in ARE-mediated decay were decreased in SS and AraCS cells (FIG. 9B-E). This includes the exosome RNA decay complex components (FIG. 9C) that are not only involved in ribosome production and other RNA processing steps (185-191) that are decreased in G0 (FIG. 2B) but are also important for mRNA and ARE mRNA decay (179, 192-198), as well as proteasome components (199-201) (FIG. 9D)—apart from key ARE-specific mRNA binding proteins that are decay or translation repression factors (FIG. 9E) (202-207). In accord with this decrease, ARE mRNA levels were increased and were translated. In addition, the key ARE binding decay factor, TTP, was phosphorylated in SS and AraCS cells (FIG. 3D-E). Phosphorylation of TTP leads to its increased mRNA and protein levels (184) and results in its inability to cause ARE-mediated decay and downregulation of gene expression; such ARE bearing, TTP targeted mRNAs are stabilized (60, 73, 75, 76, 208) and are translated (209). Consistently, we found that TTP was phosphorylated in SS and AraCS cells (FIG. 3E) and its levels were increased (FIGS. 9E, 3D). These results are consistent with our findings of increased levels and translation of ARE bearing mRNAs-including many cytokines and inflammatory factors—in SS and AraCS cells (FIG. 2-3).

Our data previously revealed an RNA binding protein FXR1—that post-transcriptionally regulates specific genes (113, 210-220)—stabilizes many ARE bearing cytokine mRNAs in early 1 day SS cells (157); consistently, we found that FXR1 depletion in early 1 day SS cells decreased the levels of ARE bearing mRNAs (FIG. 9F). FXR1 is also known to associate with ribosomes (113, 221-223) and the role of FXR1 in alternative translation has been observed to be important in cancer (224) and stem cells (225). We previously found that FXR1 also promotes translation of ARE and specific microRNA target site bearing mRNAs in 2 day SS cells via an alternate translation mechanism that is activated by such low mTOR activity-4EBP active conditions (FIG. 2B, lower panel) (32). Consistent with our current data, we found that FXR1 associated with TNFαmRNA in SS cells but not S+ cells (FIG. 3F-G), enabling TNFαexpression; depletion of FXR1 in such SS cells previously (32, 112) revealed decrease in TNFα, and many of the immune genes upregulated in SS and AraCS cells. Other factors and mRNA elements may also be involved. These results are consistent with our findings of increased expression of ARE bearing mRNAs that include cytokines and other immune genes in resistant G0 cells-due to decreased ARE decay activity and factors—as well as post-transcriptional and translational regulation by FXR1 in these conditions of reduced canonical translation (FIG. 3H).

### Example 8. DNA Damage Signaling by the ATM Pathway Transiently Activates the p38 MAPK—which Activates the Inflammatory Response Via MK2, and Interferon Pathway Via STAT1—at Early Times of Serum-Starvation and Chemotherapy Treatment—and Leads to Chemoresistance

To identify how these changes in post-transcriptional (TTP phosphorylation, FIG. 3D-E), and translational (eIF2 phosphorylation, low mTOR activity, FIG. 2A-B) mechanisms were being induced in G0 resistant cells, we examined the key signaling pathways (43) in the global profiling data from THP1 as well as other cell lines (FIG. 8E). Tumor cells show genomic instability that cause DNA damage, which can lead to DNA damage and stress signaling (40-42). DNA damage and stress signaling is also triggered by DNA damage inducing stress like serum-starvation (226) and chemotherapies that target DNA processes. DNA damage responsive ATM kinase and stress signaling can lead to downstream effects on: 1. AMPK kinase mediated mTOR inhibition as observed that inhibits canonical translation (FIG. 2A-B, 4EBP dephosphorylation), and 2. p38 MAPK stress signaling in tumor subpopulations of G0-like cells (40-43) that can lead to inhibition of canonical translation via downstream effectors—which permit non-canonical translation and increased inflammatory/immune response. Consistently, we found that the DNA damage responsive ATM kinase was activated at early times during serum-starvation or AraC treatment (4 h-1 day), which led to downstream activation, of stress-inducible p38 MAPK (FIGS. 4A-C, 10A). P38 MAPK enables a number of downstream regulators including inflammatory response genes via promoting downstream MAPKAPK2 (MK2) (64-66) that post-transcriptionally upregulates inflammatory gene expression (67-73) as well as the interferon (IFN) pathway via STAT1 (73, 82-86) (FIG. 4A). Consistent with phosphorylation and activation of p38 MAPK, we found phosphorylation of downstream effectors MK2 and STAT1 (FIGS. 4B-C, 10A).

STAT1 activation led to nuclear translocation of phospho-STAT1 (FIG. 4D), permitting its activity. In accord, IFN response genes that have been classified as IFN-related DNA damage resistance signature, (IRDS) and are associated with therapy resistance in several solid tumors (82-86) were observed to be upregulated (FIGS. 4E, 10B). Depletion of some IRDS genes decreases resistance (82, 85); therefore, this could explain in part, the resistance observed in such G0 chemoresistant cells. STAT1 increases phosphorylation of its downstream target PKR kinase (FIGS. 4B-C, 10A), explaining the increased eIF2c phosphorylation (FIG. 2B) that reduced canonical translation at the tRNA recruitment step and permits non-canonical translation. STAT1 also enhances the effect of ATM downstream signaling that inhibits mTOR activity (40-42)—by transcriptionally (227) increasing active (dephosphorylated) 4EBP1 (FIG. 2B lower panel), which inhibits canonical translation. Therefore, these downstream effects of ATM and p38 MAPK signaling led to decreased canonical translation at both rate-limiting steps of translation initiation (FIGS. 2A-B, 3), which permits non-canonical, specific mRNA translation.

MK2 activation correlated with the increased expression of inflammatory genes in SS and AraCS cells (FIG. 2E-G). Phospho-MK2 activity stabilizes TTP mRNA and leads to increased phosphorylation (67-73) and stability of the TTP protein itself (184). This correlated with the increased levels and phosphorylation of TTP in SS and AraCS cells (FIGS. 3D-E, 9E) where MK2 and TTP were phosphorylated (FIG. 4F), which prevents mRNA decay of ARE bearing mRNAs (60, 73, 75, 76, 184, 208), enabling increase of ARE bearing TNFα pro-inflammatory gene expression (FIG. 4F). Accordingly, inhibition of p38 MAPK, using the 38 MAPK inhibitor, LY2228820 (LY) (66, 228, 229), blocked phosphorylation of MK2 and thereby blocks MK2 mediated phosphorylation of TTP, and thus reduces levels of TNFα (FIG. 4F). LY also decreased phosphorylation of STAT1 and thereby of PKR that mediates eIF2α phosphorylation and inhibition. These results are consistent with our findings of increased ARE bearing, inflammatory factor mRNA levels and translation —due to decreased ARE decay activity by the ATM-p38 MAPK-MK2 axis (FIG. 2-4)—as well as translational regulation via ATM-p38 MAPK-STAT1 axis and ATM-AMPK inhibited mTOR signaling (FIGS. 4A-F, 10A-C) (45, 64, 230, 231), which reduced canonical translation and permitted previously identified (31, 32) non-canonical translation (FIG. 3F-H) in these conditions of reduced canonical translation.

### Example 9. Inhibition of Transiently Activated p38 MAPK in Early G0—at Early Times of Serum—Starvation and Chemotherapy Treatment-Prevents Chemoresistance

The distinct temporal activation of p38 MAPK and of its downstream effectors, STAT1 and MK2, at early time points of serum-starvation or AraC treatment, (FIGS. 4B-C, 10A, 4G), suggested early events that activate resistance pathways. LY treatment of cells with AraC or serum-starvation, led to inhibition of p38 MAPK activated phosphorylation of its downstream targets MK2 as well as STAT1 (FIGS. 4F, 10C, phosphorylation of total p38 MAPK did not decrease and increased as observed previously (66, 232)). To test whether the early activation of these resistance pathways is important for chemotherapy survival, leukemic cells were treated with LY before or after treatment with AraC, and then cell survival was measured using two cell viability assays and a cell death assay (FIG. 4H). As a control, S+ cells that were not treated with AraC did not show cell survival changes in response to LY in all three cell survival assays. Compared to AraC surviving cells that were exposed to only the vehicle buffer of LY, we found that cells treated with LY, one day after treatment with AraC, also did not show any significant change in cell survival (FIG. 4H). Consistently with the transient early increase in stress signaling, our data revealed that treatment with LY at early times—4 h-1 day prior to AraC treatment and along with AraC treatment-caused a much more significant decrease in cell survival compared to either AraC+vehicle control, or to LY treatment of AraC pre-treated cells (FIG. 4H). These results did not occur in non-cancerous CD34+ cells but were reproduced in other AML cells lines, where treatment with LY 4 h prior to or along with AraC (FIG. 4I), and to various concentrations of AraC (FIG. 10D), led to decreased cell survival; this was not due to effects on cell cycle status (FIG. 10E) (233). To confirm these findings, we used a second, specific inhibitor of p38 MAPK, BIRB796 (BIRB), a pan p38 MAPK inhibitor that targets all the p38 MAPK isoforms (234-238). We found that BIRB strongly decreased survival in cells that were subsequently treated with AraC but not in untreated S+ cells (FIG. 4J). As with LY, treatment with BIRB followed by treatment with AraC caused a more significant reduction in cell survival compared to either AraC alone (AraC+vehicle), or to BIRB treatment of AraC pre-treated cells (FIG. 4K). Analyses of samples treated with BIRB+AraC or AraC alone revealed that BIRB treatment blocked AraC-induced increase in phosphorylation of p38 MAPK and MK2; consistently, we found that the increased phospho-TTP upon AraC treatment alone was reduced upon BIRB co-treatment (FIG. 4L)—which would lead to decreased pro-inflammatory cytokines like TNFα. These data uncovered a stress-activated p38 MAPK/MK2 pathway that enabled chemotherapy survival through regulation of TTP, to block its activity of reducing expression of pro-survival, pro-inflammatory genes like TNFα. These studies also revealed that blocking this early-activated p38 MAPK/MK2/TTP pathway that enables chemotherapy survival through early induction of pro-survival, pro-inflammatory genes/TNFα—with inhibitors prior to and along with rather than post-chemotherapy—prevented resistance upon treatment with chemotherapy. Inhibiting these pathways after treatment with chemotherapy was less effective, given that at later time points their downstream effectors of cell survival genes are already on.

### Example 10. Inflammatory Response Genes, Such as TNFα, and Downstream NFκB-Mediated Signaling, are Upregulated in Chemoresistant G0 Cells

The DNA damage-induced ATM pathway activates p38 MAPK that further activates MK2, leading to upregulation of immune/inflammatory response genes (FIG. 5A). Inflammatory response genes ware the most predominantly upregulated category in SS and AraCS cells (FIG. 2E, 8E), indicating an important role in the maintenance of cancer G0 cells and chemoresistance. The cytokine genes upregulated in SS and AraCS cells did not significantly match the known senescence associated secretory pathway (SASP or senescence messaging secretome, SMS (239-243)) (FIG. 11A). This is consistent with differences between quiescence and senescence (3, 11), including low mTOR activity (32) and mutated p53 in these SS and AraCS THP1 cells that are quiescent (244)—and unlike the high mTOR activity and p53 roles in senescence (33). These data indicated that a quiescence- and resistance-specific set of pro-inflammatory genes are expressed in these resistant cells.

The inflammatory and immune response genes that were upregulated include cytokine mRNAs like TNFα (FIG. 5B), which we previously demonstrated was post-transcriptionally and translationally upregulated in SS G0 (32) (157), and is also upregulated in AraCS cells, along with its receptors and other inflammatory cytokines like S100A12 (FIG. 5B-C). TNFα and other cytokines can promote the NFκB pathway (245-248), which in turn, increases expression of anti-apoptotic genes—as a stress response to promote cell survival (245-250)—or can increase apoptosis (251). Our data showed an increase of NFκB signaling and anti-apoptotic genes such as BCL family members (250, 252, 253) (FIGS. 5D, 11B-C), indicating that this pro-inflammatory pathway may promote anti-apoptosis and thereby, survival and chemoresistance.

TNFα and inflammatory gene expression can be inhibited with the inflammation inhibitor, Pirfenidone (254-256) that can block TNFα levels, as well as other inflammatory factors, and is currently clinically used for inflammatory disease like fibrosis (254). Pirfenidone affects the inflammatory pathway via upstream stress induced p38 MAPKγ (49, 256). Consistently, Pirfenidone decreased the increased expression of TNFα and S100A12 (FIG. 5E-F). To test the role of inflammatory gene expression in chemoresistance, we inhibited TNFα/NFκB with anti-inflammatory drugs: 1. Pirfenidone, and 2. an NFκB inhibitor, BAY11-7082 (257), to inhibit NFκB signaling downstream of TNFα and block the cell survival/anti-apoptotic response. If this pro-inflammatory pathway was involved in chemoresistance and G0 survival, then inhibiting the pathway in conjunction with chemotherapy or serum-starvation should lead to decreased resistance and cell survival. We found that these two drugs had no effect in the absence of chemotherapy or serum-starvation in S+ cells, indicating that the concentrations used were not toxic (FIG. 5G-H). In SS or AraCS cells, co-incident treatment with Pirfenidone, which targets TNFα, had about a 40-60% decrease in cell viability in both conditions (FIG. 5G-H)—indicating that the TNFα inflammatory pathway was required for cell survival upon chemotherapy and upon serum-starvation. Consistently, we found that inhibition of TNFα/inflammatory cytokine induced downstream NFκB, with BAY11-7082, caused decreases in survival upon chemotherapy and upon serum-starvation, re-affirming that the effect of Pirfenidone is via TNFα/inflammatory pathway and downstream NFκB signaling (FIG. 5G-H).

### Example 11. Inhibition of TNFα/NFκB—Prior to or at the Same Time as Chemotherapy—Leads to Significant Decrease in Chemoresistance, Correlating with a Transient, Early Increase in Expression of TNFα/NFκB in G0

Our results in FIG. 4 with p38 MAPK indicated early induction of this cell survival pathway that leads to resistance (FIG. 4G-I). If this were true, then treating cells with anti-inflammatory Pirfenidone or BAY11-7082 after chemotherapy treatment would not reduce chemoresistance as the downstream cell survival genes that are activated by the initial surge in inflammatory response in such G0 cells would have already been induced before this later time. We tested the order of addition of inhibitors of the inflammatory pathway—either before serum-starvation or AraC treatment—or after. Consistent with these studies and our above studies in FIG. 4H, we found that using the inflammatory inhibitors, prior to or along with AraC chemotherapy, reduced cell survival and chemoresistance while treatment with inflammatory inhibitors after AraC failed to reduce resistance (FIG. 5G-H). Treatment of cells with these inhibitors, at least 18 h prior to (and continued with) AraC treatment, was significantly more effective in reducing resistance (FIG. 11D). These data indicated that activation of the inflammatory pathway was an early event in G0 resistant cells, which led to resistance, and needed to be inhibited early to prevent downstream survival regulators. Consistently, we found that TNFα expression and its downstream NFκB signaling, which was high throughout serum-starvation, was significantly increased at early times during serum-starvation (FIG. 5I).

To confirm that the chemoresistance observed was due to TNFα mediated inflammatory response that led to cell survival signaling, we constructed a THP1 cell line with a doxycycline inducible shRNA against TNFα. Induction of TNFα shRNA prior to (and continued with) treatment with AraC decreased TNFα levels and reduced cell survival upon AraC chemotherapy, compared to a control shRNA (FIG. 5J). Conversely, addition of recombinant TNFα enhanced resistance and cell survival upon subsequent AraC treatment (FIG. 5J). This was not due to cell cycle effects as TNFα treatment without subsequent AraC (FIG. 11E) did not alter the cell cycle. Consistent decrease in resistance was also observed with shRNA against S100A12 cytokine (FIG. 11F). These data suggested that the TNFα/NFκB inflammatory pathway was upregulated as an early survival pathway in G0 resistant cells, which led to downstream anti-apoptosis factors that mediated subsequent cell survival upon chemotherapy. In accord with the transient increase of the inflammatory genes in early serum-starved cells, we found that treatment with inflammation inhibitors or shRNA depletion of TNFα prior to and continued with chemotherapy was more significant in reducing chemoresistance compared to co-incident treatment—while treatment with inflammation inhibitors or TNFα shRNA after chemotherapy, failed to significantly affect resistance.

### Example 12. Inhibition of Inflammatory Response Genes Required for Chemoresistance in Leukemic Cells, Also Decreases Cell Survival Upon Chemotherapy in Breast Cancer Cells

Doxorubicin has a different mechanism compared to AraC; however, like AraC, doxorubicin targets proliferating cells at G1/S, affecting DNA replication and chromatin, and causes DNA damage response that leads to enrichment of G0 cells (258, 259). Accordingly, we found that Pirfenidone reduced chemotherapy survival in MCF7 cells treated with doxorubicin (FIG. 11G)—similar to the effect on AraC resistant THP1 cells. Consistently, in breast cancer MCF7 SS G0 cells, the inflammatory pathway was similarly increased (FIG. S2E) as in THP1 cells. These data indicated that DNA damage signaling and its downstream stress p38 MAPK signaling led to increased chemoresistance in MCF7 and THP1 cancer cells.

### Example 13. Inhibition of Inflammatory Response Genes Decreases Cell Survival Upon Clinical Therapy Treatment in Leukemic Cell Lines

Inhibition of the inflammatory pathway also decreased chemoresistance in other AML cell lines tested (FIG. 5K), indicating that this early upregulation of inflammatory factors is a general mechanism. Not all cancers showed the same response (FIG. 5K, K562 chronic myelogenous leukemia), indicating that the reduced survival was not due to general drug toxicity. Pirfenidone targets not only TNFa expression but also p38 MAPKγ and other inflammatory factors and cytokines (49, 254-256, 260); consistently, the effect of pirfenidone was greater than that of just TNFα depletion (FIG. 11H). These data correlated with early inflammation gene expression that leads to cell survival gene expression, which alter such cells to be more resistant to chemotherapy.

### Example 14. Inhibition of Specific Molecular Pathways, Upregulated in SS and AraCS Cells, Reduces Chemoresistance

Key molecular pathways (43) enriched in SS or AraCS cells (FIGS. 8E, 6A), were examined for their requirement for cell survival upon chemotherapy via drug screening of pathway inhibitors (FIG. 12A), testing for decreased cell survival with AraC (FIG. 6A-B), as conducted above with inhibition of p38 MAPK and of inflammatory genes (FIGS. 4G-I, 5F-M). As a control, S+ cells were treated with these inhibitors to ensure dosages used cause minimum toxic effects in the absence of serum-starvation or AraC treatment. Inhibitors against these pathways (FIGS. 12A, 6A) revealed that cell viability and thus resistance to chemotherapy (FIGS. 6B, 12B) was reduced with inhibition of: cholesterol biosynthesis inhibition (with Lovastatin (55-57)), apart from inflammatory response inhibition (with Pirfenidone (49, 254-256, 260) or BAY11-7082 (257)), ATM DNA damage signaling inhibition (with KU55933 (261, 262)) and stress-inducible p38 MAPK inhibition (with LY2228820 (66, 228, 229)). These data implicated DNA damage and stress pathways in chemotherapy survival, consistent with their upregulation (FIGS. 4A-4L, 10A-10E) in G0 SS and AraCS chemoresistant cells.

### Example 15. Inhibition of Cholesterol Synthesis Leads to Significant Decrease in Chemoresistance

ER stress triggers the PERK pathway to alter translation via eIF2 (109, 123, 125, 129, 132, 136, 142, 263-265), increases IRE1 activity to promote lipogenesis and stress response (266, 267), and increases cholesterol regulation via factors like SREBP2 (125, 268-270) (FIG. 6C). We found that AraC treatment and serum-starvation triggered an early response (4 hours to 1 day) of ER stress (FIG. 6D-E). This was reflected by an increase in IRE1 phosphorylation and thus activity that increased spliced XBP1 (FIG. 10A) and lipid biogenesis (FIGS. 2E, 8D), as well as a transient phosphorylation of PERK that phosphorylated eIF2c to inhibit canonical translation (FIGS. 6C-E, 2B), and an increase in the cholesterol biosynthesis and regulatory pathway (FIGS. 6D, 6A, 8D-E, 2E).

Cholesterol homeostasis genes were also increased in non-cancerous G0 HFFs (FIG. 8E) (33). This suggests that cholesterol was required in G0 for the maintenance of the G0 state, which may also contribute to its role in chemoresistance. A common goal for both G0 and chemoresistance is the inhibition of apoptosis. Cholesterol inhibitors have been observed to enable apoptosis by altering mitochondrial cholesterol (271) that inhibits pro-apoptotic caspase (55-57). Cholesterol synthesis has been predicted to also promote lipid rafts as well as intercellular signaling via vesicle production, which promote the immune/inflammatory response (50-54). Consistently, cholesterol regulatory genes and immune/inflammatory response genes (FIG. 2E) were the highest gene categories to be upregulated in G0 and chemoresistant cells.

Previous studies where cholesterol synthesis was inhibited along with chemotherapy/AraC showed reduced leukemic cell survival (51, 52, 272-274), consistent with our data in FIG. 6B where the cholesterol inhibitor lovastatin reduced cell survival upon AraC treatment. In accord with these studies, we found that lovastatin with AraC chemotherapy significantly reduced cell survival and chemoresistance in THP1 cells as well as in other AML cell lines (FIG. 6F-G). Lovastatin at higher concentrations also affected cell viability in untreated (no AraC) cells as observed previously (272) (FIGS. 6B, 12C, 6F). These data indicated that blocking the effects of cholesterol enhanced chemosensitivity and decreased survival.

### Example 16. A Combination Therapy to Block Chemoresistance

DDR induced ATM pathway (40-42) activated p38 MAPKα (44-49) that stimulates MK2 (64-66) and the interferon pathway (82-86), leading to upregulation of inflammatory response genes (67-73) (FIGS. 4A, 5A). The ER stress pathway that was also upregulated in such resistant cells further enhances the inflammatory response as well as blocks apoptosis (50-57) (FIGS. 6A, 6C-E). Use of ATM or p38 MAPKα inhibitors along with cholesterol synthesis inhibitors would therefore further augment the effect that we observed with inhibitors against downstream inflammatory factors alone (FIGS. 4G-I, 5F-M, 6F-G). Consistently, targeting either any two or all three of the above pathways prior to (and continued with) chemotherapy significantly curtailed chemoresistance. Early inhibition of leukemic cells with ATM inhibitor, KU55933, and inflammation inhibitor, Pirfenidone—or of either inhibitor with the cholesterol inhibitor, Lovastatin—had an increased combined effect when treated prior to (and continued with) chemotherapy, promoting greater reduction in resistance (FIG. 12D). Combined inhibition by KU55933 of ATM-activated DNA damage signaling and its downstream p38 MAPK pathway that can promote inflammation via MK2 and STAT1, and of inflammation by Pirfenidone (49, 254-256, 260) that targets cytokines like TNFα (254-256) and p38 MAPKγ (49, 256), prior to and along with chemotherapy led to significantly more decreases in chemoresistance. Using the clinically tested p38 MAPKα/β inhibitor, LY2228820 (66, 228, 229), instead of the ATM inhibitor that is slightly toxic to THP1 S+ cells (FIG. 6B, S+), prior to and continued with AraC chemotherapy, led to a significant, effective decrease in chemoresistance without affecting proliferating cells (FIGS. 4G-I, 6H). Inhibiting the p38 MAPK pathway with LY2228820 that targets p38 α and β isoforms (66, 228, 229), in combination with the anti-inflammatory Pirfenidone that targets TNFa translation and other inflammatory factor levels (254, 255) as well as the MAPK p38γ isoform (45, 49, 256) (which that is also key for inflammation (44, 46-48) along with p38 MAPKα and β isoforms (66, 228, 229)) led to effective loss of chemoresistance with decreased cell viability (FIG. 6H) and consistently to increased apoptosis (FIG. 6H-I) in multiple leukemic cell lines without affecting proliferating, chemotherapy-untreated cells (FIG. 6H-I). Further combination of both drugs with the cholesterol inhibitor, Lovastatin, prior to (and continued with) chemotherapy perpetuated this increased reduction in resistance (FIG. 6H). In accord with the loss of chemoresistant, G0 leukemic stem cells, colony formation was observed to be significantly reduced with cells pre-treated with the combination therapy compared to control or AraC alone treatments (FIG. J). These data indicated a more severe loss of stem cell capacity of leukemic cells treated with combination therapy compared to AraC treatment only. Consistently, phosphorylation of MK2, the target of p38 MAPK, was decreased; accordingly, TTP phosphorylation and levels decrease (184), and the levels of TNFα, which enables cell survival, were reduced much more significantly due to the combination of PFD and LY2228820 than the single drug treatments (FIG. 6K). Significantly, these results of reduced chemoresistance and tumor survival, with early and continued treatment with combination therapy of PFD and LY2228820 and AraC, substantially reduced tumors in vivo in a mouse xenograft AML (MOLM13) model, validating these findings in tumors in vivo (FIG. 6L). Importantly, we found that treatment of AML patient samples, but not non-cancerous monocytes, with combination therapy with pirfenidone and AraC decreased cell viability and AraC resistant cell survival (FIG. 6M). Thus, combinations of chemical inhibitors that target the early inflammation that triggers cell survival, when administered prior to and along with chemotherapy can effectively reduce chemoresistance in cancer cell lines in tumors in vivo and in patient tumor samples induced by DNA damage and stress signaling in subpopulations of cancer cells.

### Example 17. TTP Regulation is Required for Chemoresistance

Inhibition of the inflammatory pathway decreased chemoresistance in multiple AML cell lines tested but not all cell lines (FIG. 5K, K562). We analyzed K562 cells treated with AraC or with the combination therapy of AraC and LY and found that while phospho-MK2 was decreased, the levels of phospho-TTP (mRNA decay inactive form that cannot suppress pro-inflammatory gene/TNFα expression) was not as significantly decreased compared to THP1 and MOLM13 cells (FIG. 6N vs 6K), correlating with the lack of reduction in chemoresistance in this cell line (FIG. 5K) on inhibition of p38 MAPK/MK2 regulation of TTP. This indicated that levels of phospho-TTP and thus TTP activity (phospho-TTP regulated, decay inactive form cannot suppress pro-inflammatory gene expression (67-72, 184) (73)) was a key regulator of pro-inflammatory gene/TNFα mediated chemoresistance. To test this hypothesis, we overexpressed non-regulatable active mutant TTP that had its phosphorylation sites mutated to alanine (TTP-AA) to maintain ARE-bearing mRNA decay activity and has been shown to reduce pro-inflammatory cytokines and TNFa (60, 73, 75, 76, 184, 208). Overexpression of TTP-AA compared to control vector significantly reduced TNFα mRNA in both THP1 and K562 cells (FIG. 6N) due to the ARE-bearing mRNA decay activity of the active mutant TTP form. Consistently, we found that subsequent AraC treatment of either THP1 or of K562 cells led to decreased chemoresistance and cell viability (FIG. 6N). These data suggested that TTP phosphorylation levels and activity, mediated by stress p38 MAPK, MK2 and other signaling, enabled chemosurvival by stabilization of ARE-bearing pro-inflammatory genes that mediate cell survival, and thus chemoresistance.

### REFERENCE LIST

(1) Pardee A B. A restriction point for control of normal animal cell proliferation. Proc Natl Acad Sci USA 1974; 71:1286-90.
(2) Aragon A D, Rodriguez A L, Meirelles O, Roy S, Davidson G S, Tapia P H, et al. Characterization of differentiated quiescent and nonquiescent cells in yeast stationary-phase cultures. Mol Biol Cell 2008; 19:1271-80.
(3) Coller H A, Sang L, Roberts J M. A new description of cellular quiescence. PLoS Biol 2006; 4:e83.
(4) Chen W C, Yuan J S, Xing Y, Mitchell A, Mbong N, Popescu A C, et al. An Integrated Analysis of Heterogeneous Drug Responses in Acute Myeloid Leukemia That Enables the Discovery of Predictive Biomarkers. Cancer Res 2016; 76:1214-24.
(5) Ng S W, Mitchell A, Kennedy J A, Chen W C, McLeod J, Ibrahimova N, et al. A 17-gene stemness score for rapid determination of risk in acute leukaemia. Nature 2016; 540:433-7.
(6) Dick J E. Tumor archaeology: tracking leukemic evolution to its origins. Sci Transl Med 2014; 6:238fs23.
(7) Kreso A, Dick J E. Evolution of the cancer stem cell model. Cell Stem Cell 2014; 14:275-91.
(8) Meacham C E, Morrison S J. Tumor heterogeneity and cancer cell plasticity. Nature 2013; 501:328-37.
(9) Crews L A, Jamieson C H. Selective elimination of leukemia stem cells: hitting a moving target. Cancer Lett 2013; 338:15-22.
(10) Bhola P D, Mar B Q Lindsley R C, Ryan J A, Hogdal L J, Vo T T, et al. Functionally identifiable apoptosis-insensitive subpopulations determine chemoresistance in acute myeloid leukemia. J Clin Invest 2016; 126:3827-36.
(11) Sang L, Coller H A, Roberts J M. Control of the reversibility of cellular quiescence by the transcriptional repressor HES1. Science 2008; 321:1095-100.
(12) Tavaluc R T, Hart L S, Dicker D T, El-Deiry W S. Effects of low confluency, serum starvation and hypoxia on the side population of cancer cell lines. Cell Cycle 2007; 6:2554-62.
(13) Gupta P B, Onder T T, Jiang Q Tao K, Kuperwasser C, Weinberg R A, et al. Identification of selective inhibitors of cancer stem cells by high-throughput screening. Cell 2009; 138:645-59.
(14) Lemons J M, Feng X J, Bennett B D, Legesse-Miller A, Johnson E L, Raitman I, et al. Quiescent fibroblasts exhibit high metabolic activity. PLoS Biol 2010; 8:e1000514.
(15) Lindeman G J, Visvader J E. Insights into the cell of origin in breast cancer and breast cancer stem cells. Asia Pac J Clin Oncol 2010; 6:89-97.
(16) Salony, Sole X, Alves C P, Dey-Guha I, Ritsma L, Boukhali M, et al. AKT Inhibition Promotes Nonautonomous Cancer Cell Survival. Mol Cancer Ther 2016; 15:142-53.
(17) Dey-Guha I, Wolfer A, Yeh A C, Albeck G, Darp R, Leon E, et al. Asymmetric cancer cell division regulated by AKT. Proc Natl Acad Sci USA 2011; 108:12845-50.
(18) Zheng X, Seshire A, Ruster B, Bug G, Beissert T, Puccetti E, et al. Arsenic but not all-trans retinoic acid overcomes the aberrant stem cell capacity of PML/RARalpha-positive leukemic stem cells. Haematologica 2007; 92:323-31.
(19) Li L, Bhatia R. Stem cell quiescence. Clin Cancer Res 2011; 17:4936-41.
(20) Barnes D J, Melo J V Primitive, quiescent and difficult to kill: the role of non-proliferating stem cells in chronic myeloid leukemia. Cell Cycle 2006; 5:2862-6.
(21) Goldman J, Gordon M. Why do chronic myelogenous leukemia stem cells survive allogeneic stem cell transplantation or imatinib: does it really matter? Leuk Lymphoma 2006; 47:1-7.
(22) Reed J C. Molecular biology of chronic lymphocytic leukemia. Semin Oncol 1998; 25:11-8.
(23) Giles F J, DeAngelo D J, Baccarani M, Deininger M, Guilhot F, Hughes T, et al. Optimizing outcomes for patients with advanced disease in chronic myelogenous leukemia. Semin Oncol 2008; 35:S1-17.
(24) Krause A, Luciana M, Krause F, Rego E M. Targeting the acute myeloid leukemia stem cells. Anticancer Agents Med Chem 2010; 10:104-10.
(25) Besancon R, Valsesia-Wittmann S, Puisieux A, de Fromentel C C, Maguer-Satta V. Cancer stem cells: the emerging challenge of drug targeting. Curr Med Chem 2009; 16:394-416.
(26) Hanahan D, Weinberg R A. Hallmarks of cancer: the next generation. Cell 2011; 144:646-74.
(27) Liu H, Adler A S, Segal E, Chang H Y A Transcriptional Program Mediating Entry into Cellular Quiescence. PLoS Genet 2007; 3:e91.
(28) Sandberg R, Neilson J R, Sarma A, Sharp P A, Burge C B. Proliferating cells express mRNAs with shortened 3′ untranslated regions and fewer microRNA target sites. Science 2008; 320:1643-7.
(29) Mayr C, Bartel D P. Widespread shortening of 3′UTRs by alternative cleavage and polyadenylation activates oncogenes in cancer cells. Cell 2009; 138:673-84.
(30) Cheung T H, Rando T A. Molecular regulation of stem cell quiescence. Nat Rev Mol Cell Biol 2013; 14:10.
(31) Lee S, Truesdell S S, Bukhari S I, Lee J H, Letonqueze O, Vasudevan S. Upregulation of eIF5B controls cell-cycle arrest and specific developmental stages. Proc Natl Acad Sci USA 2014; 111:E4315-E4322.
(32) Bukhari S I, Truesdell S S, Lee S, Kollu S, Classon A, Boukhali M, et al. A Specialized Mechanism of Translation Mediated by FXR1a-Associated MicroRNP in Cellular Quiescence. Mol Cell 2016; 61:760-73.
(33) Loayza-Puch F, Drost J, Rooijers K, Lopes R, Elkon R, Agami R. p53 induces transcriptional and translational programs to suppress cell proliferation and growth. Genome Biol 2013; 14:R32.
(34) Laurenti E, Frelin C, Xie S, Ferrari R, Dunant C F, Zandi S, et al. CDK6 levels regulate quiescence exit in human hematopoietic stem cells. Cell Stem Cell 2015; 16:302-13.
(35) Ebinger S, Ozdemir E Z, Ziegenhain C, Tiedt S, Castro A C, Grunert M, et al. Characterization of Rare, Dormant, and Therapy-Resistant Cells in Acute Lymphoblastic Leukemia. Cancer Cell 2016; 30:849-62.
(36) Saito Y, Kitamura H, Hijikata A, Tomizawa-Murasawa M, Tanaka S, Takagi S, et al. Identification of therapeutic targets for quiescent, chemotherapy-resistant human leukemia stem cells. Sci Transl Med 2010; 2:17ra9.
(37) Eppert K, Takenaka K, Lechman E R, Waldron L, Nilsson B, van G P, et al. Stem cell gene expression programs influence clinical outcome in human leukemia. Nat Med 2011; 17:1086-93.
(38) Ashton J M, Balys M, Neering S J, Hassane D C, Cowley G Root D E, et al. Gene sets identified with oncogene cooperativity analysis regulate in vivo growth and survival of leukemia stem cells. Cell Stem Cell 2012; 11:359-72.
(39) Galli R, Binda E, Orfanelli U, Cipelletti B, Gritti A, De V S, et al. Isolation and characterization of tumorigenic, stem-like neural precursors from human glioblastoma. Cancer Res 2004; 64:7011-21.
(40) Shiloh Y, Ziv Y The ATM protein kinase: regulating the cellular response to genotoxic stress, and more. Nat Rev Mol Cell Biol 2013; 14:197-210.
(41) Tee A R, Proud C G DNA-damaging agents cause inactivation of translational regulators linked to mTOR signalling. Oncogene 2000; 19:3021-31.
(42) Jackson S P, Bartek J. The DNA-damage response in human biology and disease. Nature 2009; 461:1071-8.
(43) Holohan C, Van Schaeybroeck S, Longley D B, Johnston P G Cancer drug resistance: an evolving paradigm. Nat Rev Cancer 2013; 13:714-26.
(44) Qi X, Yin N, Ma S, Lepp A, Tang J, Jing W, et al. p38gamma MAPK Is a Therapeutic Target for Triple-Negative Breast Cancer by Stimulation of Cancer Stem-Like Cell Expansion. Stem Cells 2015; 33:2738-47.
(45) Wang X, McGowan C H, Zhao M, He L, Downey J S, Fearns C, et al. Involvement of the MKK6-p38+¦ Cascade in +¦-Radiation-Induced Cell Cycle Arrest. Mol Cell Biol 2000; 20:4543-52.
(46) Cuenda A, Rousseau S. p38 MAP-Kinases pathway regulation, function and role in human diseases. Biochimica et Biophysica Acta (BBA)—Molecular Cell Research 2007; 1773:1358-75.
(47) Korb A, Tohidast-Akrad M, Cetin E, Axmann R, Smolen J, Schett G Differential tissue expression and activation of p38 MAPK alpha, beta, gamma and delta isoforms in rheumatoid arthritis. Arthritis & Rheumatism 2006; 54:2745-56.
(48) Kyriakis J M, Avruch J. Mammalian mitogen-activated protein kinase signal transduction pathways activated by stress and inflammation. Physiol Rev 2001; 81:807-69.
(49) Yin N, Qi X, Tsai S, Lu Y, Basir Z, Oshima K, et al. p38[gamma] MAPK is required for inflammation-associated colon tumorigenesis. Oncogene 2016; 35:1039-48.
(50) Tall A R, Yvan-Charvet L. Cholesterol, inflammation and innate immunity. Nat Rev Immunol 2015; 15:104-16.
(51) Wong W W, Dimitroulakos J, Minden M D, Penn L Z. HMG-CoA reductase inhibitors and the malignant cell: the statin family of drugs as triggers of tumor-specific apoptosis. Leukemia 2002; 16:508-19.
(52) Benakanakere I, Johnson T, Sleightholm R, Villeda V, Arya M, Bobba R, et al. Targeting cholesterol synthesis increases chemoimmuno-sensitivity in chronic lymphocytic leukemia cells. Exp Hematol Oncol 2014; 3:24.
(53) Claudio N, Dalet A, Gatti E, Pierre P. Mapping the crossroads of immune activation and cellular stress response pathways. EMBO J 2013; 32:1214-24.
(54) Hotamisligil G S. Endoplasmic reticulum stress and the inflammatory basis of metabolic disease. Cell 2010;%19; 140:900-17.
(55) Smith B, Land H. Anticancer Activity of the Cholesterol Exporter ABCA1 Gene. Cell Reports2:580-90.
(56) Chapman-Shimshoni D, Yuklea M, Radnay J, Shapiro H, Lishner M. Simvastatin induces apoptosis of B-CLL cells by activation of mitochondrial caspase 9. Experimental Hematology 2003; 31:779-83.
(57) Montero J, Morales A, Llacuna L, Lluis J M, Terrones O, Basanez G et al. Mitochondrial cholesterol contributes to chemotherapy resistance in hepatocellular carcinoma. Cancer Res 2008; 68:5246-56.
(58) Brewer G Messenger RNA decay during aging and development. Ageing Res Rev 2002; 1:607-25.
(59) Khabar K S. Post-transcriptional control during chronic inflammation and cancer: a focus on AU-rich elements. Cell Mol Life Sci 2010; 67:2937-55.
(60) Blackshear P J. Tristetraprolin and other CCCH tandem zinc-finger proteins in the regulation of mRNA turnover. Biochem Soc Trans 2002; 30:945-52.
(61) Spasic M, Friedel C C, Schott J, Kreth J, Leppek K, Hofmann S, et al. Genome-wide assessment of AU-rich elements by the AREScore algorithm. PLoS Genet 2012; 8:e1002433.
(62) Garneau N L, Wilusz J, Wilusz C J. The highways and byways of mRNA decay. Nat Rev Mol Cell Biol 2007; 8:113-26.
(63) Damgaard C K, Lykke-Andersen J. Regulation of ARE-mRNA Stability by Cellular Signaling: Implications for Human Cancer. Cancer Treat Res 2013; 158:153-80.
(64) Reinhardt H C, Aslanian A S, Lees J A, Yaffe M B. p53 deficient cells rely on ATM and ATR-mediated checkpoint signaling through the p38 MAPK/MK2 pathway for survival after DNA damage. Cancer Cell 2007; 11:175-89.
(65) Cannell I G Merrick K A, Morandell S, Zhu C Q, Braun C J, Grant R A, et al. A Pleiotropic RNA-Binding Protein Controls Distinct Cell Cycle Checkpoints to Drive Resistance of p53-defective Tumors to Chemotherapy. Cancer Cell 2015; 28:623-37.
(66) Lalaoui N, Hafinggi K, Brumatti G Chau D, Nguyen N Y N, Vasilikos L, et al. Targeting p38 or MK2 Enhances the Anti-Leukemic Activity of Smac-Mimetics. Cancer Cell 2016; 29:145-58.
(67) Tiedje C, Holtmann H, Gaestel M. The role of mammalian MAPK signaling in regulation of cytokine mRNA stability and translation. J Interferon Cytokine Res 2014; 34:220-32.
(68) Brooks S A, Blackshear P J. Tristetraprolin (TTP): interactions with mRNA and proteins, and current thoughts on mechanisms of action. Biochim Biophys Acta 2013; 1829:666-79.
(69) Ross C R, Brennan-Laun S E, Wilson G M. Tristetraprolin: roles in cancer and senescence. Ageing Res Rev 2012; 11:473-84.
(70) Sanduja S, Blanco F F, Young L E, Kaza V, Dixon D A. The role of tristetraprolin in cancer and inflammation. Front Biosci (Landmark Ed) 2012; 17:174-88.:174-88.
(71) Sanduja S, Blanco F F, Dixon D A. The roles of TTP and BRF proteins in regulated mRNA decay. Wiley Interdiscip Rev RNA 2011; 2:42-57.
(72) Ronkina N, Menon M B, Schwermann J, Tiedje C, Hitti E, Kotlyarov A, et al. MAPKAP kinases MK2 and MK3 in inflammation: complex regulation of TNF biosynthesis via expression and phosphorylation of tristetraprolin. Biochem Pharmacol 2010; 80:1915-20.
(73) Stoecklin Q Stubbs T, Kedersha N, Wax S, Rigby W F, Blackwell T K, et al. MK2-induced tristetraprolin: 14-3-3 complexes prevent stress granule association and ARE-mRNA decay. EMBO J 2004; 23:1313-24.
(74) Stoecklin Q Stubbs T, Kedersha N, Wax S, Rigby W F, Blackwell T K, et al. MK2-induced tristetraprolin: 14-3-3 complexes prevent stress granule association and ARE-mRNA decay. EMBO J 2004; 23:1313-24.
(75) Sun L, Stoecklin Q Van W S, Hinkovska-Galcheva V, Guo R F, Anderson P, et al. Tristetraprolin (TTP)-14-3-3 complex formation protects TTP from dephosphorylation by protein phosphatase 2a and stabilizes tumor necrosis factor-alpha mRNA. J Biol Chem 2007; 282:3766-77.
(76) Clement S L, Scheckel C, Stoecklin Q Lykke-Andersen J. Phosphorylation of tristetraprolin by MK2 impairs AU-rich element mRNA decay by preventing deadenylase recruitment. Mol Cell Biol 2011; 31:256-66.
(77) Vlasova-St L, I, Bohjanen P R. Post-transcriptional regulation of cytokine and growth factor signaling in cancer. Cytokine Growth Factor Rev 2017; 33:83-93. doi: 10.1016/j.cytogfr.2016.11.004. Epub; %2016 Dec 2.:83-93.
(78) Wang H, Ding N, Guo J, Xia J, Ruan Y Dysregulation of TTP and HuR plays an important role in cancers. Tumour Biol 2016; 37:14451-61.
(79) White E J, Matsangos A E, Wilson G M. AUF 1 regulation of coding and noncoding RNA. Wiley Interdiscip Rev RNA 2017; 8:10.
(80) Khabar K S. Hallmarks of cancer and AU-rich elements. Wiley Interdiscip Rev RNA 2017; 8:10.
(81) Shen Z J, Malter J S. Regulation of AU-Rich Element RNA Binding Proteins by Phosphorylation and the Prolyl Isomerase Pinl. Biomolecules 2015; 5:412-34.
(82) Weichselbaum R R, Ishwaran H, Yoon T, Nuyten D S, Baker S W, Khodarev N, et al. An interferon-related gene signature for DNA damage resistance is a predictive marker for chemotherapy and radiation for breast cancer. Proc Natl Acad Sci USA 2008; 105:18490-5.
(83) Tsai H C, Li H, Van N L, Cai Y, Robert C, Rassool F V, et al. Transient low doses of DNA-demethylating agents exert durable antitumor effects on hematological and epithelial tumor cells. Cancer Cell 2012;%20; 21:430-46.
(84) Boelens M C, Wu T J, Nabet B Y, Xu B, Qiu Y, Yoon T, et al. Exosome transfer from stromal to breast cancer cells regulates therapy resistance pathways. Cell 2014; 159:499-513.
(85) Benci J L, Xu B, Qiu Y, Wu T J, Dada H, Twyman-Saint V C, et al. Tumor Interferon Signaling Regulates a Multigenic Resistance Program to Immune Checkpoint Blockade. Cell 2016; 167:1540-54.
(86) Duarte C W, Willey C D, Zhi D, Cui X, Harris J J, Vaughan L K, et al. Expression signature of IFN/STAT1 signaling genes predicts poor survival outcome in glioblastoma multiforme in a subtype-specific manner. PLoS One 2012; 7:e29653.
(87) de Kruijf E M, Sajet A, van Nes J G Natanov R, Putter H, Smit V T, et al. HLA-E and HLA-G expression in classical HLA class I-negative tumors is of prognostic value for clinical outcome of early breast cancer patients. J Immunol 2010; 185:7452-9.
(88) Levy E M, Bianchini M, Von Euw E M, Barrio M M, Bravo A I, Furman D, et al.

Human leukocyte antigen-E protein is overexpressed in primary human colorectal cancer. Int J Oncol 2008; 32:633-41.
(89) Strubin M, Long E O, Mach B. Two forms of the la antigen-associated invariant chain result from alternative initiations at two in-phase AUGs. Cell 1986; 47:619-25.
(90) Rock K L, Reits E, Neefjes J. Present Yourself! By MHC Class I and MHC Class II Molecules. Trends in Immunology 2016; 37:724-37.
(91) Barclay A N, Van den Berg T K. The interaction between signal regulatory protein alpha (SIRPalpha) and CD47: structure, function, and therapeutic target. Annu Rev Immunol 2014; 32:25-50. doi: 10.1146/annurev-immunol-032713-120142. Epub; %2013 Nov 6.:25-50.
(92) Kaur S, Singh S P, Elkahloun A G Wu W, Abu-Asab M S, Roberts D D. CD47-dependent immunomodulatory and angiogenic activities of extracellular vesicles produced by T cells. Matrix Biol 2014; 37:49-59. doi: 10.1016/j.matbio.2014.05.007.

Epub; %2014 Jun 2.:49-59.
(93) Sosale N Q Spinler K R, Alvey C, Discher D E. Macrophage engulfment of a cell or nanoparticle is regulated by unavoidable opsonization, a species-specific ‘Marker of’ Self CD47, and target physical properties. Curr Opin Immunol 2015; 35:107-12. doi: 10.1016/j.coi.2015.06.013. Epub; %2015 Jul 13.:107-12.
(94) Soto-Pantoja D R, Kaur S, Roberts D D. CD47 signaling pathways controlling cellular differentiation and responses to stress. Crit Rev Biochem Mol Biol 2015; 50:212-30.
(95) Zhang H, Lu H, Xiang L, Bullen J W, Zhang C, Samanta D, et al. HIF-1 regulates CD47 expression in breast cancer cells to promote evasion of phagocytosis and maintenance of cancer stem cells. Proc Natl Acad Sci USA2015; 112:E6215-E6223.
(96) McCracken M N, Cha A C, Weissman I L. Molecular Pathways: Activating T Cells after Cancer Cell Phagocytosis from Blockade of CD47 “Don't Eat Me” Signals.

Clin Cancer Res 2015; 21:3597-601.
(97) Majeti R, Chao M P, Alizadeh A A, Pang W W, Jaiswal S, Gibbs K D, et al. CD47 is an adverse prognostic factor and therapeutic antibody target on human acute myeloid leukemia stem cells. Cell 2009; 138:286-99.
(98) Imhof B A, Aurrand-Lions M. Adhesion mechanisms regulating the migration of monocytes. Nat Rev Immunol 2004; 4:432-44.
(99) Soria G Ben-Baruch A. The inflammatory chemokines CCL2 and CCL5 in breast cancer. Cancer Lett 2008; 267:271-85.
(100) Fridman W H, Pages F, Sautes-Fridman C, Galon J. The immune contexture in human tumours: impact on clinical outcome. Nat Rev Cancer 2012; 12:298-306.
(101) Borsig L, Wolf M J, Roblek M, Lorentzen A, Heikenwalder M. Inflammatory chemokines and metastasis—tracing the accessory. Oncogene 2014; %19; 33:3217-24.
(102) Ruffell B, Affara N I, Coussens L M. Differential macrophage programming in the tumor microenvironment. Trends Immunol 2012; 33:119-26.
(103) Forbes S A, Beare D, Gunasekaran P, Leung K, Bindal N, Boutselakis H, et al. COSMIC: exploring the world's knowledge of somatic mutations in human cancer. Nucleic Acids Res 2015; 43:D805-D811.
(104) Barretina J, Caponigro Q Stransky N, Venkatesan K, Margolin A A, Kim S, et al. The Cancer Cell Line Encyclopedia enables predictive modelling of anticancer drug sensitivity. Nature 2012; 483:603-7.
(105) Grant S. Ara-C: Cellular and Molecular Pharmacology. In: George F, V, editor. Advances in Cancer Research. Volume 72 ed. Academic Press; 1997. p. 197-233.
(106) Lynch R C, Medeiros B C. Chemotherapy options for previously untreated acute myeloid leukemia. Expert Opin Pharmacother 2015; 16:2149-62.
(107) DeAngelo D J, Stein E M, Ravandi F. Evolving Therapies in Acute Myeloid Leukemia: Progress at Last? Am Soc Clin Oncol Educ Book 2016; 35:e302-12. doi: 10.14694/EDBK 161258.:e302-e312.
(108) Ting L, Rad R, Gygi S P, Haas W. MS3 eliminates ratio distortion in isobaric labeling-based multiplexed quantitative proteomics. Nat Methods 2011; 8:937-40.
(109) Hsieh A C, Liu Y, Edlind M P, Ingolia N T, Janes M R, Sher A, et al. The translational landscape of mTOR signalling steers cancer initiation and metastasis. Nature 2012; 485:55-61.
(110) Truitt M L, Conn C S, Shi Z, Pang X, Tokuyasu T, Coady A M, et al. Differential Requirements for eIF4E Dose in Normal Development and Cancer. Cell 2015; 162:59-71.
(111) Gandin V, Sikstrom K, Alain T, Morita M, McLaughlan S, Larsson O, et al. Polysome fractionation and analysis of mammalian translatomes on a genome-wide scale. J Vis Exp 2014; 10.
(112) Vasudevan S, Steitz J A. AU-rich-element-mediated upregulation of translation by FXR1 and Argonaute 2. Cell 2007; 128:1105-18.
(113) Ceman S, O'Donnell W T, Reed M, Patton S, Pohl J, Warren S T. Phosphorylation influences the translation state of FMRP-associated polyribosomes. Hum Mol Genet 2003; 12:3295-305.
(114) Terenin I M, Dmitriev S E, Andreev D E, Shatsky I N. Eukaryotic translation initiation machinery can operate in a bacterial-like mode without eIF2. Nat Struct Mol Biol 2008; 15:836-41.
(115) Terenin I M, Akulich K A, Andreev D E, Polyanskaya S A, Shatsky I N, Dmitriev S E. Sliding of a 43 S ribosomal complex from the recognized AUG codon triggered by a delay in eIF2-bound GTP hydrolysis. Nucleic Acids Res 2016; 44:1882-93.
(116) Starck S R, Tsai J C, Chen K, Shodiya M, Wang L, Yahiro K, et al. Translation from the 5′ untranslated region shapes the integrated stress response. Science 2016; 351:aad3867.
(117) Holcik M. Could the eIF2+¦-Independent Translation Be the Achilles Heel of Cancer? Front Oncol 2015; 5:264.
(118) Sendoel A, Dunn J G Rodriguez E H, Naik S, Gomez N C, Hurwitz B, et al. Translation from unconventional 5′ start sites drives tumour initiation. Nature 2017; 541:494-9.
(119) Spriggs K A, Stoneley M, Bushell M, Willis A E. Re-programming of translation following cell stress allows IRES-mediated translation to predominate. Biol Cell 2008; 100:27-38.
(120) Holcik M, Sonenberg N. Translational control in stress and apoptosis. Nat Rev Mol Cell Biol 2005; 6:318-27.
(121) Zeenko V V, Wang C, Majumder M, Komar A A, Snider M D, Merrick W C, et al. An efficient in vitro translation system from mammalian cells lacking the translational inhibition caused by eIF2 phosphorylation. RNA 2008; 14:593-602.
(122) Komar A A, Mazumder B, Merrick W C. A new framework for understanding IRES-mediated translation. Gene 2012; 502:75-86.
(123) Wek R C, Jiang H Y, Anthony T G Coping with stress: eIF2 kinases and translational control. Biochem Soc Trans 2006; 34:7-11.
(124) Lorsch J R, Dever T E. Molecular view of 43 S complex formation and start site selection in eukaryotic translation initiation. J Biol Chem 2010; 285:21203-7.
(125) Ron D, Walter P. Signal integration in the endoplasmic reticulum unfolded protein response. Nat Rev Mol Cell Biol 2007; 8:519-29.
(126) Zismanov V, Chichkov V, Colangelo V, Jamet Sn, Wang S, Syme A, et al. Phosphorylation of eIF2&# x3b1; Is a Translational Control Mechanism Regulating Muscle Stem Cell Quiescence and Self-Renewal. Cell Stem Cell 18:79-90.
(127) Thoreen C C, Chantranupong L, Keys H R, Wang T, Gray N S, Sabatini D M. A unifying model for mTORC 1-mediated regulation of mRNA translation. Nature 2012; 485:109-13.
(128) Sonenberg N, Hinnebusch A G Regulation of translation initiation in eukaryotes: mechanisms and biological targets. Cell 2009; 136:731-45.
(129) Hinnebusch A G The Scanning Mechanism of Eukaryotic Translation Initiation. Annu Rev Biochem 2014.
(130) Fonseca B D, Smith E M, Yelle N, Alain T, Bushell M, Pause A. The ever-evolving role of mTOR in translation. Semin Cell Dev Biol 2014; 36:102-12. doi: 10.1016/j.semcdb.2014.09.014. Epub; %2014 Sep 27.:102-12.
(131) Culjkovic B, Topisirovic I, Borden K L. Controlling gene expression through RNA regulons: the role of the eukaryotic translation initiation factor eIF4E. Cell Cycle 2007; 6:65-9.
(132) Silvera D, Formenti S C, Schneider R J. Translational control in cancer. Nat Rev Cancer 2010; 10:254-66.
(133) Gray N K, Wickens M. Control of translation initiation in animals. Annu Rev Cell Dev Biol 1998; 14:399-458.
(134) Bjur E, Larsson O, Yurchenko E, Zheng L, Gandin V, Topisirovic I, et al. Distinct translational control in CD4+ T cell subsets. PLoS Genet 2013; 9:e1003494.
(135) Hornstein E, Tang H, Meyuhas O. Mitogenic and nutritional signals are transduced into translational efficiency of TOP mRNAs. Cold Spring Harb Symp Quant Biol 2001; 66:477-84.
(136) Han K, Jaimovich A, Dey G Ruggero D, Meyuhas O, Sonenberg N, et al. Parallel measurement of dynamic changes in translation rates in single cells. Nat Methods 2014; 11:86-93.
(137) Thoreen C C. The molecular basis of mTORC 1-regulated translation. Biochem Soc Trans 2017; 45:213.
(138) Miloslavski R, Cohen E, Avraham A, Iluz Y, Hayouka Z, Kasir J, et al. Oxygen sufficiency controls TOP mRNA translation via the TSC-Rheb-mTOR pathway in a 4E-BP-independent manner. J Mol Cell Biol 2014; 6:255-66.
(139) Ivanov P, Kedersha N, Anderson P. Stress puts TIA on TOP. Genes Dev 2011; 25:2119-24.
(140) Fonseca B D, Zakaria C, Jia J J, Graber T E, Svitkin Y, Tahmasebi S, et al. La-related Protein 1 (LARP 1) Represses Terminal Oligopyrimidine (TOP) mRNA Translation Downstream of mTOR Complex 1 (mTORC 1). J Biol Chem 2015; 290:15996-6020.
(141) Damgaard C K, Lykke-Andersen J. Translational coregulation of 5Ç¦TOP mRNAs by TIA-1 and TIAR. Genes Dev 2011; 25:2057-68.
(142) Thakor N, Holcik M. IRES-mediated translation of cellular messenger RNA operates in elF2alpha-independent manner during stress. Nucleic Acids Res 2012; 40:541-52.
(143) Arava Y, Wang Y, Storey J D, Liu C L, Brown P O, Herschlag D. Genome-wide analysis of mRNA translation profiles in Saccharomyces cerevisiae. PNAS 2003; 100:3889-94.
(144) Piques M, Schulze W X, Hohne M, Usadel Br, Gibon Y, Rohwer J, et al. Ribosome and transcript copy numbers, polysome occupancy and enzyme dynamics in Arabidopsis. Mol Syst Biol 2009; 5:314.
(145) Liu M J, Wu S H, Chen H M, Wu S H. Widespread translational control contributes to the regulation of Arabidopsis photomorphogenesis. Mol Syst Biol 2012; 8:566.
(146) Meads M B, Gatenby R A, Dalton W S. Environment-mediated drug resistance: a major contributor to minimal residual disease. Nat Rev Cancer 2009; 9:665-74.
(147) McMillin D W, Negri J M, Mitsiades C S. The role of tumour-stromal interactions in modifying drug response: challenges and opportunities. Nat Rev Drug Discov 2013; 12:217-28.
(148) Miller P G Al-Shahrour F, Hartwell K A, Chu L P, Jaras M, Puram R V, et al. In Vivo RNAi screening identifies a leukemia-specific dependence on integrin beta 3 signaling. Cancer Cell 2013; 24:45-58.
(149) Chen M B, Lamar J M, Li R, Hynes R O, Kamm R D. Elucidation of the Roles of Tumor Integrin beta1 in the Extravasation Stage of the Metastasis Cascade. Cancer Res 2016; 76:2513-24.
(150) Oudin M J, Jonas O, Kosciuk T, Broye L C, Guido B C, Wyckoff J, et al. Tumor Cell-Driven Extracellular Matrix Remodeling Drives Haptotaxis during Metastatic Progression. Cancer Discov 2016; 6:516-31.
(151) Naba A, Clauser K R, Ding H, Whittaker C A, Carr S A, Hynes R O. The extracellular matrix: Tools and insights for the “omics” era. Matrix Biol 2016; 49:10-24. doi: 10.1016/j.matbio.2015.06.003. Epub; 2015 Jul 8.:10-24.
(152) Baumeister S H, Freeman G J, Dranoff Q Sharpe A H. Coinhibitory Pathways in Immunotherapy for Cancer. Annu Rev Immunol 2016; 20; 34:539-73. doi: 10.1146/annurev-immunol-032414-112049. Epub; 2016 Feb 25.:539-73.
(153) Shiao S L, Ganesan A P, Rugo H S, Coussens L M. Immune microenvironments in solid tumors: new targets for therapy. Genes Dev 2011; 25:2559-72.
(154) Medler T R, Cotechini T, Coussens L M. Immune response to cancer therapy: mounting an effective antitumor response and mechanisms of resistance. Trends Cancer 2015; 1:66-75.
(155) Palucka A K, Coussens L M. The Basis of Oncoimmunology. Cell 2016; 164:1233-47.
(156) Shukla S A, Rooney M S, Rajasagi M, Tiao Q Dixon P M, Lawrence M S, et al. Comprehensive analysis of cancer-associated somatic mutations in class I HLA genes. Nat Biotechnol 2015; 33:1152-8.
(157) Le T O, Kollu S, Lee S, Al-Salah M, Truesdell S S, Vasudevan S. Regulation of monocyte induced cell migration by the RNA binding protein, FXR1. Cell Cycle 2016; 15:1874-82.
(158) Landon A L, Muniandy P A, Shetty A C, Lehrmann E, Volpon L, Houng S, et al. MNKs act as a regulatory switch for eIF4E1 and eIF4E3 driven mRNA translation in DLBCL. Nat Commun 2014; 5:5413. doi: 10.1038/ncomms6413.:5413.
(159) Khabar KSA. Hallmarks of cancer and AUÇÉrich elements. Wiley Interdiscip Rev RNA 2017; 8:e1368.
(160) Zhang T, Kruys V, Huez G Gueydan C. AU-rich element-mediated translational control: complexity and multiple activities of trans-activating factors. Biochem Soc Trans 2001; 30:952-8.
(161) Vlasova-St L, I, Bohjanen P R. Post-transcriptional regulation of cytokine and growth factor signaling in cancer. Cytokine Growth Factor Rev 2017; 33:83-93. doi: 10.1016/j.cytogfr.2016.11.004. Epub; 2016 Dec 2.:83-93.
(162) Griseri P, Pages G Control of pro-angiogenic cytokine mRNA half-life in cancer: the role of AU-rich elements and associated proteins. J Interferon Cytokine Res 2014; 34:242-54.
(163) Perez-Ortin J E, Alepuz P, Chavez S, Choder M. Eukaryotic mRNA decay: methodologies, pathways, and links to other stages of gene expression. J Mol Biol 2013; 425:3750-75.
(164) Moore A E, Young L E, Dixon D A. MicroRNA and AU-rich element regulation of prostaglandin synthesis. Cancer Metastasis Rev 2011; 30:419-35.
(165) Schott J, Stoecklin G Networks controlling mRNA decay in the immune system. Wiley Interdiscip Rev RNA 2010; 1:432-56.
(166) Berkovits B D, Mayr C. Alternative 3′ UTRs act as scaffolds to regulate membrane protein localization. Nature 2015; 522:363-7.
(167) Castelli E C, Veiga-Castelli L C, Yaghi L, Moreau P, Donadi E A. Transcriptional and Posttranscriptional Regulations of the HLA-G Gene. J Immunol Res 2014; 2014:734068.
(168) Sharma S, Verma S, Vasudevan M, Samanta S, Thakur J K, Kulshreshtha R. The interplay of HuR and miR-3134 in regulation of A U rich transcriptome. RNA Biol 2013; 10:1283-90.
(169) Tserel L, Runnel T, Kisand K, Pihlap M, Bakhoff L, Kolde R, et al. MicroRNA expression profiles of human blood monocyte-derived dendritic cells and macrophages reveal miR-511 as putative positive regulator of Toll-like receptor 4. J Biol Chem 2011; 286:26487-95.
(170) Nichols R C, Botson J, Wang X W, Hamilton B J, Collins J E, Uribe V, et al. A flexible approach to studying post-transcriptional gene regulation in stably transfected mammalian cells. Mol Biotechnol 2011; 48:210-7.
(171) Lin C C, Liu L Z, Addison J B, Wonderlin W F, Ivanov A V, Ruppert J M. A KLF4-miRNA-206 Autoregulatory Feedback Loop Can Promote or Inhibit Protein Translation Depending upon Cell Context. Mol Cell Biol 2011; 31:2513-27.
(172) Vasudevan S. Posttranscriptional Upregulation by MicroRNAs. 2011.doi: 10.1002/wrna. 121. Wiley Interdiscip Rev RNA 2011.
(173) Espel E. The role of the AU-rich elements of mRNAs in controlling translation. Semin Cell Dev Biol 2005; 16:59-67.
(174) Mazan-Mamczarz K, Galban S, De Silanes I L, Martindale J L, Atasoy U, Keene J D, et al. RNA-binding protein HuR enhances p53 translation in response to ultraviolet light irradiation. Proc Natl Acad Sci USA 2003; 100:8354-9.
(175) Mukherjee N, Corcoran D L, Nusbaum J D, Reid D W, Georgiev S, Hafner M, et al. Integrative Regulatory Mapping Indicates that the RNA-Binding Protein HuR Couples Pre-mRNA Processing and mRNA Stability. Mol Cell 2011; 43:327-39.
(176) Wang W, Furneaux H, Cheng H, Caldwell M C, Hutter D, Liu Y, et al. HuR regulates p21 mRNA stabilization by U V light. Mol Cell Biol 2000; 20:760-9.
(177) Wang W, Fan J, Yang X, Furer-Galban S, Lopez dS, I, von Kobbe C, et al. AMP-activated kinase regulates cytoplasmic HuR. Mol Cell Biol 2002; 22:3425-36.
(178) Ambrosino C, Mace G Galban S, Fritsch C, Vintersten K, Black E, et al. Negative feedback regulation of MKK6 mRNA stability by p38alpha mitogen-activated protein kinase. Mol Cell Biol 2003; 23:370-81.
(179) Lal A, Mazan-Mamczarz K, Kawai T, Yang X, Martindale J L, Gorospe M. Concurrent versus individual binding of HuR and AUF 1 to common labile target mRNAs. EMBO J 2004; 23:3092-102.
(180) Lal A, Navarro F, Maher C A, Maliszewski L E, Yan N, O'Day E, et al. miR-24 Inhibits cell proliferation by targeting E2F2, MYC, and other cell-cycle genes via binding to “seedless” 3′UTR microRNA recognition elements. Mol Cell 2009; 35:610-25.
(181) Srikantan S, Abdelmohsen K, Lee E K, Tominaga K, Subaran S S, Kuwano Y, et al. Translational control of Top2A influences doxorubicin efficacy. Mol Cell Biol 2011.
(182) Tominaga K, Srikantan S, Lee E K, Subaran S S, Martindale J L, Abdelmohsen K, et al. Competitive regulation of Nucleolin expression by HuR and miR-494. Mol Cell Biol 2011.
(183) Neininger A, Kontoyiannis D, Kotlyarov A, Winzen R, Eckert R, Volk H D, et al. MK2 targets AU-rich elements and regulates biosynthesis of tumor necrosis factor and interleukin-6 independently at different post-transcriptional levels. J Biol Chem 2002; 277:3065-8.
(184) Hitti E, Iakovleva T, Brook M, Deppenmeier S, Gruber A D, Radzioch D, et al. Mitogen-activated protein kinase-activated protein kinase 2 regulates tumor necrosis factor mRNA stability and translation mainly by altering tristetraprolin expression, stability, and binding to adenine/uridine-rich element. Mol Cell Biol 2006; 26:2399-407.
(185) Zinder J C, Lima C D. Targeting RNA for processing or destruction by the eukaryotic RNA exosome and its cofactors. Genes Dev 2017; 31:88-100.
(186) Schneider C, Tollervey D. Threading the barrel of the RNA exosome. Trends Biochem Sci 2013; 38:485-93.
(187) Schmidt K, Butler J S. Nuclear RNA surveillance: role of TRAMP in controlling exosome specificity. Wiley Interdiscip Rev RNA 2013; 4:217-31.
(188) Schaeffer D, Clark A, Klauer A A, Tsanova B, van H A. Functions of the cytoplasmic exosome. Adv Exp Med Biol 2011; 702:79-90. doi: 10.1007/978-1-4419-7841-77.:79-90.
(189) Lykke-Andersen S, Brodersen D E, Jensen T H. Origins and activities of the eukaryotic exosome. J Cell Sci 2009; 122:1487-94.
(190) Lorentzen E, Basquin J, Conti E. Structural organization of the RNA-degrading exosome. Curr Opin Struct Biol 2008; 18:709-13.
(191) Raijmakers R, Schilders G Pruijn G J. The exosome, a molecular machine for controlled RNA degradation in both nucleus and cytoplasm. Eur J Cell Biol 2004; 83:175-83.
(192) Tiedje C, Holtmann H, Gaestel M. The role of mammalian MAPK signaling in regulation of cytokine mRNA stability and translation. J Interferon Cytokine Res 2014; 34:220-32.
(193) Hau H H, Walsh R J, Ogilvie R L, Williams D A, Reilly C S, Bohjanen P R. Tristetraprolin recruits functional mRNA decay complexes to ARE sequences. J Cell Biochem 2007; 100:1477-92.
(194) Gherzi R, Lee K Y, Briata P, Wegmuller D, Moroni C, Karin M, et al. A K H domain RNA binding protein, KSRP, promotes ARE-directed mRNA turnover by recruiting the degradation machinery. Mol Cell 2004; 14:571-83.
(195) Mukherjee D, Gao M, O'Connor J P, Raijmakers R, Pruijn G, Lutz C S, et al. The mammalian exosome mediates the efficient degradation of mRNAs that contain AU-rich elements. EMBO J 2002; 21:165-74.
(196) Chen C Y, Gherzi R, Ong S E, Chan E L, Raijmakers R, Pruijn G J, et al. A U binding proteins recruit the exosome to degrade ARE-containing mRNAs. Cell 2001; 107:451-64.
(197) Nechama M, Peng Y, Bell O, Briata P, Gherzi R, Schoenberg D R, et al. KSRP-PMR1-exosome association determines parathyroid hormone mRNA levels and stability in transfected cells. BMC Cell Biol 2009; 10:70. doi: 10.1186/1471-2121-10-70.:70-10.
(198) Esnault S, Shen Z J, Whitesel E, Malter J S. The peptidyl-prolyl isomerase Pinl regulates granulocyte-macrophage colony-stimulating factor mRNA stability in T lymphocytes. J Immunol 2006; 177:6999-7006.
(199) Laroia Q Cuesta R, Brewer Q Schneider R J. Control of mRNA decay by heat shock-ubiquitin-proteasome pathway. Science 1999; 284:499-502.
(200) Laroia Q Sarkar B, Schneider R J. Ubiquitin-dependent mechanism regulates rapid turnover of AU-rich cytokine mRNAs. Proc Natl Acad Sci USA 2002; 99:1842-6.
(201) Brooks S A. Functional interactions between mRNA turnover and surveillance and the ubiquitin proteasome system. Wiley Interdiscip Rev RNA 2010; 1:240-52.
(202) Moore A E, Chenette D M, Larkin L C, Schneider R J. Physiological networks and disease functions of RNA-binding protein AUF 1. Wiley Interdiscip Rev RNA 2014; 5:549-64.
(203) Simone L E, Keene J D. Mechanisms coordinating ELAV/Hu mRNA regulons. Curr Opin Genet Dev 2013; 23:35-43.
(204) White E J, Brewer Q Wilson G M. Post-transcriptional control of gene expression by AUFI: mechanisms, physiological targets, and regulation. Biochim Biophys Acta 2013; 1829:680-8.
(205) Abdelmohsen K, Gorospe M. RNA-binding protein nucleolin in disease. RNA Biol 2012; 9:799-808.
(206) von R C, Di M S, Mazroui R, Gallouzi I E. Turnover of AU-rich-containing mRNAs during stress: a matter of survival. Wiley Interdiscip Rev RNA 2011; 2:336-47.
(207) Soller M, Li M, Haussmann I U. Determinants of ELAV gene-specific regulation. Biochem Soc Trans 2010; 38:1122-4.
(208) Tiedje C, Diaz-Munoz M D, Trulley P, Ahlfors H, Laa+¦ K, Blackshear P J, et al. The RNA-binding protein TTP is a global post-transcriptional regulator of feedback control in inflammation. Nucleic Acids Research 2016; 44:7418-40.
(209) Tiedje C, Kotlyarov A, Gaestel M. Molecular mechanisms of phosphorylation-regulated TTP (tristetraprolin) action and screening for further TTP-interacting proteins. Biochem Soc Trans 2010; 38:1632-7.
(210) Jin P, Zarnescu D C, Ceman S, Nakamoto M, Mowrey J, Jongens T A, et al. Biochemical and genetic interaction between the fragile X mental retardation protein and the microRNA pathway. Nat Neurosci 2004; 7:113-7.
(211) Kirkpatrick L L, McIlwain K A, Nelson D L. Comparative genomic sequence analysis of the FXR gene family: FMR1, FXR1, and FXR2. Genomics 2001; 78:169-77.
(212) Xu X L, Zong R, Li Z, Biswas M H, Fang Z, Nelson D L, et al. FXR1P but not FMRP regulates the levels of mammalian brain-specific microRNA-9 and microRNA-124. J Neurosci 2011; 31:13705-9.
(213) Khandjian E W, Bardoni B, Corbin F, Sittler A, Giroux S, Heitz D, et al. Novel isoforms of the fragile X related protein FXR1P are expressed during myogenesis. Hum Mol Genet 1998; 7:2121-8.
(214) Dube M, Huot M E, Khandjian E W. Muscle specific fragile X related protein 1 isoforms are sequestered in the nucleus of undifferentiated myoblast. BMC Genet 2000; 1:4.
(215) Mazroui R, Huot M E, Tremblay S, Filion C, Labelle Y, Khandjian E W. Trapping of messenger RNA by Fragile X Mental Retardation protein into cytoplasmic granules induces translation repression. Hum Mol Genet 2002; 11:3007-17.
(216) Garnon J, Lachance C, Di Marco S, Hel Z, Marion D, Ruiz M C, et al. Fragile X-related protein FXR1P regulates proinflammatory cytokine tumor necrosis factor expression at the post-transcriptional level. J Biol Chem 2005; 280:5750-63.
(217) Bechara E G Didiot M C, Melko M, Davidovic L, Bensaid M, Martin P, et al. A novel function for fragile X mental retardation protein in translational activation. PLoS Biol 2009; 7:e16.
(218) Davidovic L, Durand N, Khalfallah O, Tabet R, Barbry P, Mari B, et al. A novel role for the RNA-binding protein FXR1P in myoblasts cell-cycle progression by modulating p21/Cdknla/Cip1/Wafl mRNA stability. PLoS Genet 2013; 9:e1003367.
(219) Darnell J C, Fraser C E, Mostovetsky O, Darnell R B. Discrimination of common and unique RNA-binding activities among Fragile X mental retardation protein paralogs. Hum Mol Genet 2009; 18:3164-77.
(220) Ascano M, Jr., Mukherjee N, Bandaru P, Miller J B, Nusbaum J D, Corcoran D L, et al. FMRP targets distinct mRNA sequence elements to regulate protein expression. Nature 2012; 492:382-6.
(221) Zhang Y, O'Connor J P, Siomi M C, Srinivasan S, Dutra A, Nussbaum R L, et al. The fragile X mental retardation syndrome protein interacts with novel homologs FXR1 and FXR2. EMBO J 1995; 14:5358-66.
(222) Siomi M C, Zhang Y, Siomi H, Dreyfuss G Specific sequences in the fragile X syndrome protein FMR1 and the FXR proteins mediate their binding to 60S ribosomal subunits and the interactions among them. Mol Cell Biol 1996; 16:3825-32.
(223) Ishizuka A, Siomi M C, Siomi H. A Drosophila fragile X protein interacts with components of RNAi and ribosomal proteins. Genes Dev 2002; 16:2497-508.
(224) Qian J, Hassanein M, Hoeksema M D, Harris B K, Zou Y, Chen H, et al. The RNA binding protein FXR1 is a new driver in the 3q26-29 amplicon and predicts poor prognosis in human cancers. Proc Natl Acad Sci USA2015; 112:3469-74.
(225) Sugiyama H, Takahashi K, Yamamoto T, Iwasaki M, Narita M, Nakamura M, et al. Natl promotes translation of specific proteins that induce differentiation of mouse embryonic stem cells. Proc Natl Acad Sci USA 2017; 114:340-5.
(226) Shi Y, Felley-Bosco E, Marti™, Orlowski K, Pruschy M, Stahel R A. Starvation-induced activation of ATM/Chk2/p53 signaling sensitizes cancer cells to cisplatin. BMC Cancer 2012; 12:571.
(227) Wang S, Patsis C, Koromilas A E. Statl stimulates cap-independent mRNA translation to inhibit cell proliferation and promote survival in response to antitumor drugs. Proc Natl Acad Sci USA 2015; 112:E2149-E2155.
(228) Patnaik A, Haluska P, Tolcher A W, Erlichman C, Papadopoulos K P, Lensing J L, et al. A First-in-Human Phase I Study of the Oral p38 MAPK Inhibitor, Ralimetinib (LY2228820 Dimesylate), in Patients with Advanced Cancer. Clin Cancer Res 2016; 22:1095.
(229) Mader M, de Dios A, Shih C, Bonjouklian R, Li T, White W, et al. Imidazolyl benzimidazoles and imidazo[4,5-b]pyridines as potent p38 alpha MAP kinase inhibitors with excellent in vivo antiinflammatory properties. Bioorganic & Medicinal Chemistry Letters 2008; 18:179-83.
(230) de Nadal E, Ammerer Q Posas F. Controlling gene expression in response to stress. Nat Rev Genet 2011; 12:833-45.
(231) Raman M, Earnest S, Zhang K, Zhao Y, Cobb M H. TAO kinases mediate activation of p38 in response to DNA damage. EMBO J 2007; 26:2005-14.
(232) Tate C, Blosser W, Wyss L, Evans Q Xue Q, Pan Y, et al. LY2228820 Dimesylate, a Selective Inhibitor of p38 Mitogen-activated Protein Kinase, Reduces Angiogenic Endothelial Cord Formation in Vitro and in Vivo. Journal of Biological Chemistry 2013; 288:6743-53.
(233) Thornton™, Rincon M. Non-Classical P38 Map Kinase Functions: Cell Cycle Checkpoints and Survival. Int J Biol Sci 2009; 5:44-52.
(234) Pargellis C, Tong L, Churchill L, Cirillo P F, Gilmore T, Graham A G et al. Inhibition of p38 MAP kinase by utilizing a novel allosteric binding site. Nat Struct Biol 2002; 9:268-72.
(235) Yasui H, Hideshima T, Ikeda H, Jin J, Ocio E M, Kiziltepe T, et al. BIRB 796 enhances cytotoxicity triggered by bortezomib, heat shock protein (Hsp) 90 inhibitor, and dexamethasone via inhibition of p38 mitogen-activated protein kinase/Hsp27 pathway in multiple myeloma cell lines and inhibits paracrine tumour growth. Br J Haematol 2007; 136:414-23.
(236) Regan J, Breitfelder S, Cirillo P, Gilmore T, Graham A G Hickey E, et al. Pyrazole urea-based inhibitors of p38 MAP kinase: from lead compound to clinical candidate. J Med Chem 2002; 45:2994-3008.
(237) Kuma Y, Sabio Q Bain J, Shpiro N, Marquez R, Cuenda A. BIRB796 inhibits all p38 MAPK isoforms in vitro and in vivo. J Biol Chem 2005; 280 (20): 19472-9.
(238) Regan J, Capolino A, Cirillo P F, Gilmore T, Graham A G Hickey E, et al. Structure-activity relationships of the p38alpha MAP kinase inhibitor 1-(5-tert-butyl-2-p-tolyl-2H-pyrazol-3-yl)-3-[4-(2-morpholin-4-yl-ethoxy)naph-thalen-1-yl]urea (BIRB 796). J Med Chem 2003; 46:4676-86.
(239) Campisi J, d'Adda di F F. Cellular senescence: when bad things happen to good cells. Nat Rev Mol Cell Biol 2007; 8:729-40.
(240) Serrano M, Lin A W, McCurrach M E, Beach D, Lowe S W. Oncogenic ras provokes premature cell senescence associated with accumulation of p53 and pl6INK4a. Cell 1997; 88:593-602.
(241) Coppe J P, Desprez P Y, Krtolica A, Campisi J. The senescence-associated secretory phenotype: the dark side of tumor suppression. Annu Rev Pathol 2010; 5:99-118. doi: 10.1146/annurev-pathol-121808-102144.:99-118.
(242) Salama R, Sadaie M, Hoare M, Narita M. Cellular senescence and its effector programs. Genes Dev 2014; 28:99-114.
(243) Kuilman T, Peeper D S. Senescence-messaging secretome: SMS-ing cellular stress. Nat Rev Cancer 2009; 9:81-94.
(244) Durland-Busbice S, Reisman D. Lack of p53 expression in human myeloid leukemias is not due to mutations in transcriptional regulatory regions of the gene. Leukemia 2002; 16:2165-7.
(245) Kagoya Y, Yoshimi A, Kataoka K, Nakagawa M, Kumano K, Arai S, et al. Positive feedback between N F-kappaB and TNF-alpha promotes leukemia-initiating cell capacity. J Clin Invest 2014; 124:528-42.
(246) Volk A, Li J, Xin J, You D, Zhang J, Liu X, et al. Co-inhibition of N F-kappaB and JNK is synergistic in TNF-expressing human AML. J Exp Med 2014; 211:1093-108.
(247) Zhou X, Zhou S, Li B, Li Q, Gao L, Li D, et al. Transmembrane TNF-+¦ preferentially expressed by leukemia stem cells and blasts is a potent target for antibody therapy. Blood 2015.
(248) Frelin C, Imbert V+, Griessinger E, Peyron A C, Rochet N, Philip P, et al. Targeting NF-+¦ B activation via pharmacologic inhibition of IKK2-induced apoptosis of human acute myeloid leukemia cells. Blood 2005; 105:804.
(249) el-Ghissassi F, Valsesia-Wittmann S, Falette N, Duriez C, Walden P D, Puisieux A. BTG2 (TIS21/PC3) induces neuronal differentiation and prevents apoptosis of terminally differentiated PC12 cells. Oncogene 2002; 21:6772-8.
(250) Chang T P, Vancurova I. Bcl3 regulates pro-survival and pro-inflammatory gene expression in cutaneous T-cell lymphoma. Biochim Biophys Acta 2014; 1843:2620-30.
(251) Hoesel B, Schmid J A. The complexity of NF-+¦ B signaling in inflammation and cancer. Molecular Cancer 2013; 12:86.
(252) Haq R, Yokoyama S, Hawryluk E B, J+¦nsson GrB, Frederick D T, McHenry K, et al. BCL2A1 is a lineage-specific antiapoptotic melanoma oncogene that confers resistance to BRAF inhibition. PNAS 2013; 110:4321-6.
(253) Kurosu T, Fukuda T, Miki T, Miura O. BCL6 overexpression prevents increase in reactive oxygen species and inhibits apoptosis induced by chemotherapeutic reagents in B-cell lymphoma cells. Oncogene 2003; 22:4459-68.
(254) Grattendick K J, Nakashima J M, Feng L, Girl S N, Margolin S B. Effects of three anti-TNF-alpha drugs: etanercept, infliximab and pirfenidone on release of TNF-alpha in medium and TNF-alpha associated with the cell in vitro. Int Immunopharmacol 2008; 8:679-87.
(255) Nakazato H, Oku H, Yamane S, Tsuruta Y, Suzuki R. A novel anti-fibrotic agent pirfenidone suppresses tumor necrosis factor-alpha at the translational level. European Journal of Pharmacology 2002; 446:177-85.
(256) Ozes O, Blatt L M, Seiwert S D. Use of pirfenidone in therapeutic regimens. U.S. Pat. No. 7,407,973. B2, 1-46. 8-May 2008. Ref Type: Generic (257) Rushworth S A, Bowles K M, Raninga P, MacEwan D J. N F-kappaB-inhibited acute myeloid leukemia cells are rescued from apoptosis by heme oxygenase-1 induction. Cancer Res 2010; 70:2973-83.
(258) Xu F, Wang F, Yang T, Sheng Y, Zhong T, Chen Y Differential drug resistance acquisition to doxorubicin and paclitaxel in breast cancer cells. Cancer Cell International 2014; 14:538.
(259) Tyson D R, Garbett S P, Frick P L, Quaranta V. Fractional proliferation: a method to deconvolve cell population dynamics from single-cell data. Nat Methods 2012; 9:923-8.
(260) Schaefer C J, Ruhrmund D W, Pan L, Seiwert S D, Kossen K. Antifibrotic activities of pirfenidone in animal models. European Respiratory Review 2011; 20:85.
(261) Hickson I, Zhao Y, Richardson C J, Green S J, Martin N M, Orr A I, et al. Identification and characterization of a novel and specific inhibitor of the ataxia-telangiectasia mutated kinase ATM. Cancer Res 2004; 64:9152-9.
(262) Ivanov V N, Zhou H, Partridge M A, Hei T K. Inhibition of ataxia telangiectasia mutated kinase activity enhances TRAIL-mediated apoptosis in human melanoma cells. Cancer Res 2009; 69:3510-9.
(263) Muranen T, Selfors L M, Worster D T, Iwanicki M P, Song L, Morales F C, et al. Inhibition of PI3K/mTOR leads to adaptive resistance in matrix-attached cancer cells. Cancer Cell 2012; 21:227-39.
(264) Liberman N, Gandin V, Svitkin Y V, David M, Virgili Gv, Jaramillo M, et al. DAP5 associates with eIF2+¦ and eIF4AI to promote Internal Ribosome Entry Site driven translation. Nucleic Acids Research 2015.
(265) Holcik M, Sonenberg N. Translational control in stress and apoptosis. Nat Rev Mol Cell Biol 2005; 6:318-27.
(266) Sha H, He Y, Yang L, Qi L. Stressed out about obesity: IRE1&# x3b1;&# x2013;XBP1 in metabolic disorders. Trends in Endocrinology & Metabolism22:374-81.
(267) Grootjans J, Kaser A, Kaufman R J, Blumberg R S. The unfolded protein response in immunity and inflammation. Nat Rev Immunol 2016; 16:469-84.
(268) Senft D, Ronai Z A. UPR, autophagy, and mitochondria crosstalk underlies the E R stress response. Trends Biochem Sci 2015; 40:141-8.
(269) Tsai Y C, Weissman A M. The Unfolded Protein Response, Degradation from the Endoplasmic Reticulum, and Cancer. Genes Cancer 2010; 1:764-78.
(270) Colgan S M, Tang D, Werstuck G H, Austin R C. Endoplasmic reticulum stress causes the activation of sterol regulatory element binding protein-2. The international journal of biochemistry & cell biology 2007; 39:1843-51.
(271) Smith B, Land H. Anticancer activity of the cholesterol exporter ABCA1 gene. Cell Rep 2012; 2:580-90.
(272) Li H Y, Appelbaum F R, Willman C L, Zager R A, Banker D E. Cholesterol-modulating agents kill acute myeloid leukemia cells and sensitize them to therapeutics by blocking adaptive cholesterol responses. Blood 2003; 101:3628-34.
(273) Lishner M, Bar-SefA, Elis A, Fabian I. Effect of simvastatin alone and in combination with cytosine arabinoside on the proliferation of myeloid leukemia cell lines. J Investig Med 2001; 49:319-24.
(274) Holstein S A, Hohl R J. Interaction of cytosine arabinoside and lovastatin in human leukemia cells. Leukemia Research25:651-60.
(275) Zhang J, Harrison J S, Studzinski G P. Isoforms of p38MAPK gamma and delta contribute to differentiation of human AML cells induced by 1,25-dihydroxyvitamin D (3). Exp Cell Res 2011; 317:117-30.
(276) Muaddi H, Majumder M, Peidis P, Papadakis A I, Holcik M, Scheuner D, et al. Phosphorylation of elF2alpha at serine 51 is an important determinant of cell survival and adaptation to glucose deficiency. Mol Biol Cell 2010; 21:3220-31.
(277) Anderson P, Kedersha N. Stressful initiations. J Cell Sci 2002; 115:3227-34.
(278) Borden K L, Culjkovic-Kraljacic B. Ribavirin as an anti-cancer therapy: acute myeloid leukemia and beyond? Leuk Lymphoma 2010; 51:1805-15.
(279) Walters B, Thompson S R. Cap-Independent Translational Control of Carcinogenesis. Front Oncol 2016; 6:128.
(280) Miskimins W K, Wang G Hawkinson M, Miskimins R. Control of Cyclin-Dependent Kinase Inhibitor p27 Expression by Cap-Independent Translation. Mol Cell Biol 2001; 21:4960-7.
(281) Jiang H, Coleman J, Miskimins R, Srinivasan R, Miskimins W K. Cap-independent translation through the p27 5GÇ¦-UTR. Nucleic Acids Res 2007; 35:4767-78.
(282) Cuesta R, Mart+ [symbol] nez-S+ínchez A, Gebauer F+. miR-181a Regulates Cap-Dependent Translation of p27 (kip1) mRNA in Myeloid Cells. Mol Cell Biol 2009; 29:2841-51.
(283) Truitt M L, Ruggero D. New frontiers in translational control of the cancer genome. Nat Rev Cancer 2016; 16:288-304.
(284) Crews L A, Jiang Q, Zipeto M A, Lazzari E, Court A C, Ali S, et al. An RNA editing fingerprint of cancer stem cell reprogramming. J Transl Med 2015; 13:52. doi: 10.1186/s12967-014-0370-3.:52-0370.
(285) Crews L A, Balaian L, Delos Santos N P, Leu H S, Court A C, Lazzari E, et al. RNA Splicing Modulation Selectively Impairs Leukemia Stem Cell Maintenance in Secondary Human AML. Cell Stem Cell 2016; 19:599-612.
(286) Kharas M G, Lengner C J, Al-Shahrour F, Bullinger L, Ball B, Zaidi S, et al. Musashi-2 regulates normal hematopoiesis and promotes aggressive myeloid leukemia. Nat Med 2010; 16:903-8.
(287) Jaffrey S R, Kharas M G Emerging links between m6A and misregulated mRNA methylation in cancer. Genome Med 2017; 9:2-0395.
(288) Graubert T A, Shen D, Ding L, Okeyo-Owuor T, Lunn C L, Shao J, et al. Recurrent mutations in the U2AF1 splicing factor in myelodysplastic syndromes. Nat Genet 2011; 44:53-7.
(289) Wang L, Brooks A N, Fan J, Wan Y, Gambe R, Li S, et al. Transcriptomic Characterization of SF3B1 Mutation Reveals Its Pleiotropic Effects in Chronic Lymphocytic Leukemia. Cancer Cell 2016; 30:750-63.
(290) Griseri P, Bourcier C, Hieblot C, Essafi-Benkhadir K, Chamorey E, Touriol C, et al. A synonymous polymorphism of the Tristetraprolin (TTP) gene, an AU-rich mRNA-binding protein, affects translation efficiency and response to Herceptin treatment in breast cancer patients. Hum Mol Genet 2011; 20:4556-68.
(291) Brennan S E, Kuwano Y, Alkharouf N, Blackshear P J, Gorospe M, Wilson G M. The mRNA-destabilizing protein tristetraprolin is suppressed in many cancers, altering tumorigenic phenotypes and patient prognosis. Cancer Res 2009; 69:5168-76.
(292) Carrick D M, Blackshear P J. Comparative expression of tristetraprolin (TTP) family member transcripts in normal human tissues and cancer cell lines. Arch Biochem Biophys 2007; 462:278-85.
(293) Stoecklin G Gross B, Ming X F, Moroni C. A novel mechanism of tumor suppression by destabilizing AU-rich growth factor mRNA. Oncogene 2003; 22:3554-61.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing resistance to chemotherapy in a subject who has cancer, the method comprising administering to the subject an effective amount of a p38 MAPK inhibitor prior to administering chemotherapy.

2. The method of claim 1, wherein the cancer is acute myelogenous leukemia (AML).

3. The method of claim 1, wherein the p38 MAPK inhibitor is selected from the group consisting of SB203580; Doramapimod (BIRB 796); SB202190 (FHPI); Ralimetinib (LY2228820); VX-702; PH-797804; VX-745; TAK-715; Pamapimod (R-1503, Ro4402257); BMS-582949; SB239063; Losmapimod (GW856553X); Skepinone-L; Pexmetinib (ARRY-614); Hydroxyquinazoline; AL 8697; AMG 548; AMG-47a; ARRY-797; CGH 2466 dihydrochloride; CMPD-1; CV-65; D4476 DBM 1285 dihydrochloride; EO 1428; JX-401; Losmapimod/GW856533X; ML 3403; p38/SAPK2 Inhibitor (SB 202190); Pamapimod; PD 169,316; R1487; Saquayamycin B1; SB 202190; SB 203580; SB 706504; SB202190 Hydrochloride); SB203580; SB220025; SB239063; SB242235; SCIO-323, SCIO 469; SD-169; SKF 86002 dihydrochloride; SX 011; TA 01; TA 02; TAK 715; VX-745; or VX-702.

4. The method of claim 1, further comprising administering an effective amount of pirfenidone with the p38 MAPK inhibitor.

5. The method of claim 1, wherein at least one dose of the p38 MAPK inhibitor is administered 2-24 hours before a first dose of chemotherapy, and optionally wherein additional doses of the p38 MAPK inhibitor are administered concurrently with (e.g., at the same time as, or 1-24 hours before, each additional dose of) chemotherapy.

6. A method of identifying whether a subject who has cancer is in need of a treatment for reducing resistance to chemotherapy, the method comprising:
providing a sample comprising cells from the cancer in the subject;
detecting a level of phosphorylated Tristetraprolin (phospho-TTP) in the sample;
comparing the level of phospho-TTP in the sample to a reference level of phospho-TTP;
identifying a subject who has a level of phospho-TTP above the reference level as being in need of a treatment for reducing resistance to chemotherapy, and
optionally administering to the subject an effective amount of a p38 MAPK inhibitor prior to administering chemotherapy.

7. The method of claim 6, wherein the cancer is acute myelogenous leukemia (AML).

8. The method of claim 6, wherein the p38 MAPK inhibitor is selected from the group consisting of SB203580; Doramapimod (BIRB 796); SB202190 (FHPI); Ralimetinib (LY2228820); VX-702; PH-797804; VX-745; TAK-715; Pamapimod (R-1503, Ro4402257); BMS-582949; SB239063; Losmapimod (GW856553X); Skepinone-L; Pexmetinib (ARRY-614); Hydroxyquinazoline; AL 8697; AMG 548; AMG-47a; ARRY-797; CGH 2466 dihydrochloride; CMPD-1; CV-65; D4476 DBM 1285 dihydrochloride; EO 1428; JX-401; Losmapimod/GW856533X; ML 3403; p38/SAPK2 Inhibitor (SB 202190); Pamapimod; PD 169,316; R1487; Saquayamycin B1; SB 202190; SB 203580; SB 706504; SB202190 Hydrochloride); SB203580; SB220025; SB239063; SB242235; SCIO-323, SCIO 469; SD-169; SKF 86002 dihydrochloride; SX 011; TA 01; TA 02; TAK 715; VX-745; or VX-702.

9. The method of claim 6, further comprising administering an effective amount of pirfenidone with the p38 MAPK inhibitor.

10. The method of claim 6, wherein the p38 MAPK inhibitor is administered 2-24 hours before the chemotherapy.

11. A pharmaceutical composition comprising a p38MAPK inhibitor and pirfenidone, and a pharmaceutically acceptable carrier.

12. The pharmaceutical composition of claim 11, wherein the p38 MAPK inhibitor is Ralimetinib (LY2228820).

13.-15. (canceled)
